(19) United States
(12) Patent Application Publication
(10) Pub. No.: US 2024/0245602 A1
(43) Pub. Date: Jul. 25, 2024

(54) DRUG COMBINATION KITS AND METHODS OF DRUG DELIVERY

(71) Applicants: President and Fellows of Harvard College, Cambridge, MA (US); Massachusetts Eye and Eye Infirmary, Boston, MA (US)

(72) Inventors: Ida PAVLICHENKO, Cambridge, MA (US); Joanna AIZENBERG, Cambridge, MA (US); Haritosh PATEL, Cambridge, MA (US); Michael AIZENBERG, Cambridge, MA (US); Cathy ZHANG, Cambridge, MA (US); Aaron Kyle REMENSCHNEIDER, Boston, MA (US); Elliot D. KOZIN, Boston, MA (US)

(73) Assignees: President and Fellows of Harvard College, Cambridge, MA (US); Massachusetts Eye and Ear Infirmary, Boston, MA (US)

(21) Appl. No.: 18/023,566

(22) PCT Filed: Aug. 27, 2021
(86) PCT No.: PCT/US2021/048060

§ 371 Date: Feb. 27, 2023

Related U.S. Application Data
(60) Provisional application No. 63/072,029, filed on Aug. 28, 2020.

(51) Int. Cl. A61K 9/00; A61F 11/00; A61P 27/16

(52) U.S. Cl. CPC A61K 9/0046; A61F 11/00; A61P 27/16

(57) ABSTRACT

A kit for delivering a drug formulation to treat disorders, e.g. car disorders, is disclosed. The kit includes two components: a drug formulation and a conduit for delivering the drug formulation. The kit may contain a tailored drug formulation that includes one or more of a therapeutic agent, a priming agent, an activating agent, and a reversal agent. In certain embodiments, the conduit may be provided with additional therapeutic formulations preloaded into the conduit. In some embodiments, a plug can be provided over the inner surface of the conduit where the plug contains a plug formulation.

### CROSS-REFERENCE TO RELATED APPLICATIONS

This application is a National Stage Entry of PCT International Application No. PCT/US2021/48060 filed Aug. 27, 2021, which claims the benefit of priority to U.S. Provisional Application No. 63/072,029, entitled “Drug Combination Kits and Methods of Drug Delivery in Otic Diseases” filed on Aug. 28, 2020, the disclosure of which is hereby incorporated by reference in its entirety.

### TECHNICAL FIELD

The present application relates to a drug delivery device and method. More specifically, the application relates to devices and methods for the treatment of middle ear and/or inner ear disorders, and applicable to treatment of other diseases in the human body.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

In order to treat middle and inner ear disorders, it is often desirable to administer therapeutic agents, e.g., medications or other medical fluids, into the middle and inner ear of a patient, and the delivery of medicines to such structures is often of primary importance. Typical clinical practice involves either oral, venous, or arterial drug delivery, or topical drug delivery. Topical access to the inner ear may be achieved through a variety of middle-inner ear interface tissue structures, such as the tympanic membrane, the round window membrane, the oval window/stapes footplate, the annual ligament or the endolymphatic sac/endolymphatic duct. Similarly, topical access is of high priority for administering therapeutic agents, into other parts of parts of human body.

### SUMMARY

Currently available drug delivery methods for middle and inner ear diseases carry the risk of attendant side effects. They can be invasive and risky, require multiple daily doses (e.g., intratympanic injections or infusions) of drugs, can cause patient discomfort and non-compliance, and may introduce infections or other immune disorders as a result of microbial or endotoxin presence, resulting in permanent structural damage (e.g., perforation of the tympanic membrane) or in hearing loss and the like. Thus, an objective of embodiments of this disclosure is to develop a drug delivery method of providing a) therapeutically effective concentrations of therapeutic agents in the middle and inner ear over a prolonged or any other desired period of time, and b) a fast and effective drug delivery at a desired time.

Certain embodiments of the present disclosure describe a kit for delivering a therapeutic agent to treat disorders. In some embodiments, the kit is configured to deliver therapeutic agent to treat ear disorders. The kit includes two components: a drug formulation and a conduit. The drug formulation includes one or more of a therapeutic agent A-d, a priming agent B-d, an activating agent C-d, and a reversal agent D-d. The priming agent B-d modifies one or more properties of the conduit from a first configuration to a second configuration either upon activation of the priming agent B-d or passively by wetting and/or infusion of the priming agent B-d. The activating agent C-d is capable of releasing the therapeutic agent A-d toward a portion of an inner surface of the conduit upon activation of the activating agent C-d. The reversal agent D-d reverses one or more properties of the conduit from the second configuration to the first configuration upon activation of the reversal agent D-d.

In certain embodiment, the one or more of the properties include: physical properties, chemical properties, mechanical properties, optical properties, thermal properties, acoustical properties, electrical properties, magnetic properties, geometry, or combinations thereof.

In certain embodiments, the physical properties include wettability, drag, physical heterogeneity, surface energy, surface lubricity, porosity, texture, permeability, swellability, anisotropy, acoustic properties, or combinations thereof; the chemical properties include type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties include Young's moduli, elasticity, stiffness, hardness, toughness, friction, rigidity, compressibility, sound absorption, or combinations thereof; the optical properties include light absorption, light scattering, transmission, or combinations thereof; the thermal properties include glass transition temperature, thermal conductivity, thermal expansion, or combinations thereof; the acoustical properties include acoustical absorption, speed of sound, sound reflection, sound transfer, elasticity, or combinations thereof, for purposes of fluid transport driven by Surface Acoustic Waves (SAW); the electrical properties include capacitance, dielectric constant, resistivity, conductivity, susceptibility, permittivity, piezoelectric constant, or combinations thereof, for electrically driven transport; the magnetic properties include Curie temperature, diamagnetism, hall coefficient, hysteresis, magnetoresistance, permeability, piezomagnetism, spin hall effect, or combinations thereof, for magnetically driven flow; and the geometry includes size, shape, orifice geometry, profile, curvature, or combinations thereof.

In certain embodiments, the kit further includes a therapeutic formulation preloaded into the conduit. The therapeutic formulation includes one or more of a therapeutic agent A-t, a priming agent B-t, an activating agent C-t, and a reversal agent D-t.

In certain embodiments, the therapeutic agent A-d and/or the therapeutic agent A-t is selected from the group consisting of anti-inflammatory, anti-allergic, and analgesic agents, non-steroidal anti-inflammatory (NSAID) agents, tranquilizing agents, corticosteroids, volume expanding agents, vasodilating agents, antihistaminic agents, anticholinergic agents, vaccines, adjuvants, enzymes, monoclonal antibodies, drug dissolution and disintegration promoters, exosomes, microcrystals, solid lipid nanoparticles, preservatives, antifoams, taste-masking agents, chelating agents, buffering agents, bulking agents, anti-coagulants, antiviral agents, immunosuppressive agents, antacids and H2-blockers, antiemetics, calcium channel blockers, anticancer agents, vitamins, silk, vascular rheologic agents, neuroprotective agents, neuromodulators; anti-apoptotic agents; antiseptics, therapeutic peptides, therapeutic polysaccharides, hormones, free radical scavengers, neurotrophins, fungicides, antibodies, antigens, bacteriocides, urea, mannitol, sorbitol, glycerol, lidocaine, xylocaine, epinephrine, immunoglobulins, sodium chloride, heparin, hyaluronidase, aminoglycoside neurotrophins, therapeutic peptides, therapeutic polysaccharides, cortico-steroids, vitamins, cortisone, analgesics, antipyrine, benzocaine, procaine, antioxidants, methionine, N-acetylcysteine, trolox, neurotrophins, GDNF, BDNF, anti-apoptotic agents, anti-necrotic agents, leupeptin or combinations thereof, fentanyl citrate; salicylates; droperidol, prochlorperazine; fluocinolone, dexamethasone, prednisone; batahistine, niacin and papaverine; meclizine, dimenhydrinate, scopolamine, and promethazine; glycopyrrolate, propantheline, and atropine; ofloxacin, ciprofloxacin, finafloxacin ampicillin, cefuroxime, amoxicillin and ceftriaxone; cyclophosphomide and cyclosporine; thiazide, triamterene and carbonic anhydrase inhibitors; nizatidine and cimetidine; metoclopramide or diphenidol; diltiazem, nifedipine and verapamil; gentian violet, cresylate, mercurochrome, chloramphenicol, polymyxin; tolnaftate, nystatin, clotrimazole, miconazole; arylcycloalkyamine, ketamine, antibiotics, neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, oat extract (avenanthramide), and combinations thereof.

In certain embodiments, the therapeutic agent A-d and/or the therapeutic agent A-t is selected from the group consisting of neomycin B, kanamycin A, kanamycin B, erythromycin and azithromycin, vancomycin, gentamicin, amikacin), dibekacin, tobramycin, streptomycin, paromomycin, sisomicin, butirosin, geneticin, isepamicin, and netilmicin, gentamicins A, C1, C1, C2 and D; neomycins B and C, hybrimycin, propikacin (UK 31214), ribostamycin, seldomycin, trehalosamine, D-mannosyl-D-glucosaminide, apramycin, bluensomycin, netromycin, tobramycin, sisomicin, destomycin, Antibiotic A-396-I, dibekacin, kasugamycin, fortimicin, or derivatives; calcineurin inhibitors, interleukin Inhibitors, TNF-α inhibitors, platelet activating factor antagonists, nitric oxide synthase inhibitors, histamine antagonists, estrogen-related receptor beta modulators, cyclosporin A (CsA) and CsA derivatives, voclosporin, tacrolimus) and derivatives, ascomycin, pimecrolimus (SDZ AM 981, 33-epi-chlor-33desoxy-ascomycin), L-732,531 (32-O-(1-hydroxyethylindol-5-yl)-ascomycin), L-685,818 (FK506BD) and V-10,367; FMPP (4-(fluoromethyl)phenyl phosphate), tyrphostins, norcantharidin, okadaic acid, endothall, kaempferol, barbiturates, 1,5-dibenzoyloxymethyl-norcantharidin, gossypol, Lie120, PD144795, diberufin, dipyridamole, NCI3, INCA compounds, BTPs or 3,5-bis(trifluoromethyl)pyrazoles, BTP1, BTP2 (YM-58483), BTP3, BTP A-285222, ST1959, AM404, UR-1505, Triflusal, rocoglamide derivatives, WIN 53071, trifluoroperazine, KRM-III, caffeic acid phenyl ethyl ester (CAPE), YM-53792, quinazolinediones, pyrrolopyrimidinediones, NFAT-68, NFAT-133, punicalagin, imperatorin, quinolone alkaloids, 2-aminopyrimidine derivatives compound, pyrimidine derivatives compound, quinazoline derivatives compound, quinazoline sulfonamide compound, bicyclic heteroaryl-substituted imidazole compound, thieno-pyrimidine compound, furo-pyrimidine compound and mequitazine compound, 1-[(5-chloro-1H-indol-2-yl)carbonyl]-4-methylpiperazine or 4-((3R)-3-Aminopyrrolidin-1-yl)-6,7-dihydro-5H-benzo[6,7]cyclohepta[1,2-d]pyrimidin-2-ylamine or cis-4-(Piperazin-1-yl)-5,6,7a,8,9,10,11,11a-octahydrobenzofuro[2,3-h]quinazolin-2-amine or 7-(furan-2-yl)-4-(piperazin-1-yl)quinazolin-2-amine or 1-(7-(2-amino-6-(4-methylpiperazin-1-yl) pyrimidin-4-yl)-3,4-dihdroisoquinolin-2(1H)-yl)-2-cyclopentylethanone or 1-[(5-Chloro-1H-benzimidazol-2-yl)carbonyl]-4-methylpiperazine maleate or PF-3893787 or PF-3893787-18 or JNJ39758979 or UR-63325, antisense RNA or DNA molecules, small inhibitory RNAs (siRNAs), short hairpin RNA, micro RNA, DNAzymes, modified or synthetic DNA or RNA degradation-resistant polynucleoside amides, peptide nucleic acids (PNAs), locked nucleic acids (LNAs) and other nucleobase-containing polymers, 1H-pyrrole-2,5-dione compounds, 1,5-dihydro-2H-pyrrol-2-one compounds, 3-(pyridin-2-yl)-1H-indol-2-ol based compounds, 2-pyrimidinylaminoethylamino-2-pyridyl containing compounds, a carboxy alkyl ester, a quinic acid derivative, a caffeic acid derivative, a ferulic acid derivative, or a quinic acid lactone or derivative thereof or pharmaceutically acceptable salt thereof, and combinations thereof.

In certain embodiments, the therapeutic agent A-d and/or the therapeutic agent A-t is selected from the group consisting of thiazide, triamterene, or carbonic anhydrase; diazepam, meclizine, dimenhydrinate, prochlorperazine, promethazine, and preazepam; glycopyrrolate, propantheline, atropine, scopolamine, and combinations thereof.

In certain embodiments, the therapeutic agent A-d and/or the therapeutic agent A-t is selected from the group consisting of probiotics L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, and S. thermophiles, and combinations thereof.

In certain embodiments, the priming agent B-d and/or the priming agent B-t are selected from the group consisting of carrier liquid for the therapeutic agent, surfactants, low-surface-energy liquids, lubricating agents, acids and bases, emulsifiers, rheology control agents, flow-promoting agents, extenders, defoaming agents, plasticizers, thickeners, heat stabilizers, porogens, levelling agents, anti-cratering agents, fillers, UV absorbers, curing agents, diluents, adjuvants, buffering agents, moistening agents, anti-oxidants, oxidizing or reducing agents, mono-dentate or multi-dentate or chelating ligands, crosslinking agents, solubilizing agents, organic and inorganic solvents, SPION, cubosomes, polymerosomes, preservatives, polyglycols, long chain hydroxy polyanionic polysaccharides, long chain nitrogen containing polymers, long chain hydroxy polyanionic polysaccharides, glycerol esters, glycerol ethers, glycols, glycolesters and glycolethers, glycerol, monoacetin, diacetin and diacetone alcohol, poly(lactams), polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, waxes, organic co-solvents, alkylene glycols, ethoxylated or propoxylated alcohols, amines, waterless colloid silica, povidone, talc, monolein, magnesium stearate, flavouring agents, lactose, glucose, sucrose, starches, cellulose, derivatives of cellulose, tragacanth, malt, talc, partially or fully fluorinated liquids, oils, cocoa butter, animal fat, vegetable fats, paraffins, silicones, bentonites, silicic acid, zinc oxide, glycols, polyols, esters, agar, buffering agents, alginic acid, pyrogen-free water, isotonic saline, Ringer's solution, ethyl alcohol, phosphate buffer solutions, acacia, colloidal silicon dioxide, calcium glycerophosphate, calcium lactate, maltodextrin, glycerine, magnesium silicate, cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, propylene glycol, dimethyl isosorbide, polyoxyethylene fatty acid glycerides, vegetable oils, polyoxyethylene alkylethers, alkylphenyl ethers, and combinations thereof.

In certain embodiments, the activating agent C-d and/or the activating agent C-t are selected from the group consisting of accelerators, acid scavengers, activators, adhesion promoters, antiblocking agents, antifogging agents, antimicrobials, catalysts, coagulants, colorants, curing agents, thermal stabilizers, viscosity control agents, oxidizing or reducing agents, mono-dentate or multi-dentate or chelating ligands, solubilizing agents, organic and inorganic solvents, acids and bases, pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilising compounds, aliphatic compounds, aldehydes, UV absorbers, photoswitchable molecules, photochromic compounds, spiropyrans, azobenzenes, diarylethenes, fulgides, overcrowded alkenes, plasmonic, photothermal or magnetic nanoparticles, catalysts, enzymes, agents capable of extracting therapeutic components from the conduit, and combinations thereof.

In certain embodiments, the reversal agent D-d and/or the reversal agent D-t are selected from the group consisting of stoichiometric reagents, chelators, Ringer's solution, saline solution, water, oils, liquid crystal molecules, thermoresponsive molecules, supramolecules, biomolecules, antibodies, antigens, fluorophores, sterols, surfactants, and combinations thereof.

In certain embodiments, the kit further includes a lubricating fluid formed over at least a portion of the inner surface and the outer surface of the conduit.

In certain embodiments, the lubricating fluid is selected from the group consisting of castor oil, silicone oils, fluocinolone acetonide oil, mineral oil, fully or partially fluorinated hydrocarbons, organosilicone compound, silicones, tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, fully, partially or non-fluorinated poly- and oligoethers, fully, partially or non-fluorinated poly- and oligoesters, fully, partially or non-fluorinated poly- and oligoisocyanates, lecithin, glycerin, lipid emulsions, peanut oil, cottonseed oil, safflower oil, sesame oil, olive oil, corn oil and soybean oil, almond oil, borage oil, black currant seed oil, castor oil, corn oil, safflower oil, rapeseed oil, coconut oil, palm oil, canola oil, tea tree oil, and combinations thereof.

In certain embodiments, the therapeutic formulation is embedded in the conduit between the inner surface and the outer surface.

In certain embodiments, the kit further includes a plug over the inner surface of the conduit. The plug includes a plug formulation, and the plug formulation comprises one or more of a therapeutic agent A-p, a priming agent B-p, an activating agent C-p, and a reversal agent D-p.

In certain embodiments, any one of the priming agent B-d, the priming agent B-t, or the priming agent B-p is the carrier liquid for any one of the therapeutic agent A-d, the therapeutic agent A-t, or the therapeutic agent A-p.

In certain embodiments, one or more of the priming agent B-d, the priming agent B-t, and the priming agent B-p is a low-surface-tension liquid.

In certain embodiments, one or more of the priming agent B-d, the priming agent B-t, and the priming agent B-p is the lubricating fluid.

In certain embodiments, one or more of the priming agent B-d, the priming agent B-t, and the priming agent B-p is silicone oil.

In certain embodiments, one or more of the therapeutic agent A-d, the priming agent B-d, the activating agent C-d, and the reversal agent D-d are administered sequentially.

In certain embodiments, the kit includes the A-d and the D-d. The A-d is deposited onto the conduit, and the D-d is applied to modify or close the conduit subsequent to transport of the A-d to prevent penetration by environmental water and ototoxic compounds.

In certain embodiments, the kit includes the A-d, the C-d, and the D-d. The A-d, the C-d, and the D-d are administered in a sequential order, wherein the A-d is deposited into the conduit, the C-d stimulates the progression of the A-d through the conduit, and the D-d is applied to modify or close the conduit subsequent to transport of the A-d to prevent penetration by environmental water and ototoxic compounds.

In certain embodiments, the kit includes the A-d, the C-d, and the D-d. The C-d, the A-d, and the D-d are administered in a sequential order, wherein deposition of the C-d allows penetration of the A-d to be triggered, and the D-d is applied to modify or close the conduit subsequent to transport of the A-d to prevent penetration by environmental water and ototoxic compounds.

In certain embodiments, the kit includes the A-d, the B-d, and the D-d. The B-d, the A-d, and the D-d are administered in a sequential order, wherein the B-d is applied to promote transport of the A-d, and the D-d is applied to modify or close the conduit subsequent to transport of the A-d to prevent penetration by environmental water and ototoxic compounds.

In certain embodiments, the kit includes the A-d, the B-d, the C-d and the D-d. The B-d, the C-d, the A-d, and the D-d are administered in a sequential order, wherein the B-d is applied to promote transport of the C-d, the C-d is triggered to allow penetration of the A-d, and the D-d is applied to modify or close the conduit subsequent to transport of the A-d to prevent penetration by environmental water and ototoxic compounds.

In certain embodiments, the kit includes the A-d; the A-d is deposited onto the conduit.

In certain embodiments, the kit includes the A-d and the C-d. The A-d and the C-d are administered in a sequential order, wherein the A-d is deposited into the conduit, and the C-d stimulates the progression of the A-d through the conduit.

In certain embodiments, the kit includes the A-d and the C-d. The C-d and the A-d are administered in a sequential order: deposition of the C-d allows penetration of the A-d to be triggered.

In certain embodiments, the kit includes the A-d and the B-d. The B-d and the A-d are administered in a sequential order, wherein the B-d is applied to promote transport of the A-d.

In certain embodiments, the kit includes the A-d, the B-d, and the C-d. The B-d, the C-d, and the A-d are administered in a sequential order, wherein the B-d is applied to promote transport of the C-d, and the C-d is triggered to allow penetration of the A-d.

In certain embodiments, the kit includes the A-d and the D-t. The A-d is deposited onto the conduit, and the D-t is eluted or released to modify or close the conduit subsequent to transport of the A-d to prevent penetration by environmental water and ototoxic compounds.

In certain embodiments, the kit includes the A-d, the C-d, and the D-t. The A-d, the C-d, and the D-t are administered in a sequential order, wherein the A-d is deposited into the conduit, the C-d stimulates the progression of the A-d through the conduit, and the D-t is eluted or released to modify or close the conduit subsequent to transport of the A-d to prevent penetration by environmental water and ototoxic compounds.

In certain embodiments, the kit includes the A-d, the C-d, and the D-t. The C-d, the A-d, and the D-d are administered in a sequential order, wherein deposition of the C-d allows penetration of the A-d to be triggered, and the D-t is eluted or released to modify or close the conduit subsequent to transport of the A-d to prevent penetration by environmental water and ototoxic compounds.

In certain embodiments, the kit includes the A-d, the B-d, and the D-t. The B-d, and the A-d are administered in a sequential order, wherein the B-d is applied to promote transport of the A-d, and the D-t is eluted or released to modify or close the conduit subsequent to transport of the A-d to prevent penetration by environmental water and ototoxic compounds.

In certain embodiments, the kit includes the A-d, the B-d, the C-d and the D-t. The B-d, the C-d, and the A-d are administered in a sequential order, wherein the B-d is applied to promote transport of the C-d, the C-d is triggered to allow penetration of the A-d, and the D-t is eluted or released to modify or close the conduit subsequent to transport of the A-d to prevent penetration by environmental water and ototoxic compounds.

In certain embodiments, the kit includes the A-t, the B-d, and the D-d. The B-d, and the D-d are administered in a sequential order, wherein the B-d enables A-t to desorb, cleave or elute from the conduit, and the D-d is applied to modify or close the conduit subsequent to transport of the A-t to prevent penetration by environmental water and ototoxic compounds.

In certain embodiments, the kit includes the A-t, the C-d, and the D-d. The C-d, and the D-d are administered in a sequential order, wherein the C-d triggers the A-t to be cleaved, decomposed, or eluted from the conduit, and the D-d is applied to modify or close the conduit subsequent to transport of the A-t to prevent penetration by environmental water and ototoxic compounds.

In certain embodiments, the kit includes the A-t, the B-d, the C-d, and the D-d. The B-d, the C-d, and the D-d are administered in a sequential order, wherein the B-d is applied to promote transport of the C-d, the C-d is triggered to enable release of the A-t, and the D-d is applied to modify or close the conduit subsequent to transport of the A-t to prevent penetration by environmental water and ototoxic compounds.

In certain embodiments, the kit includes the A-t, the B-d, the C-d, and the D-d. The C-d, the B-d, and the D-d are administered in a sequential order, wherein the C-d is applied to promote transport of the B-d, the B-d is triggered to enable release of the A-t, and the D-d is applied to modify or close the conduit subsequent to transport of the A-t to prevent penetration by environmental water and ototoxic compounds.

In certain embodiments, the A-t is reapplied to become absorbed into the conduit again.

In certain embodiments, the kit includes the A-t, the B-d, and the C-d. The C-d, and the B-d are administered in a sequential order, wherein the C-d is applied to promote transport of the B-d, the B-d is triggered to enable release of the A-t.

In certain embodiments, the kit includes the A-d, the B-t, and the C-t. The A-d is administered, wherein activation of the C-t causes the B-t to elute, and the B-t that is eluted promotes transport of the A-d through the conduit.

In certain embodiments, the kit includes the A-d, the B-t, the C-d, and the C-t. The A-d, and the C-d are administered in a sequential order, wherein activation of the C-t causes the B-t to desorb or elute from the conduit, the B-t that is desorbed or eluted, in conjunction with a stimulus provided by the C-d, enables transport of the A-d through the conduit.

In certain embodiments, the kit includes the A-d, the B-d, the B-t, the C-d and the C-t. The A-d, the B-d, and the C-d are administered in a sequential order, wherein the B-d is administered in the lumen to release the B-t and the C-t that facilitate transport of the initially applied A-d.

In certain embodiments, the kit further includes D-d. The D-d is administered after the C-d, wherein the D-d is applied to modify or close the conduit subsequent to transport of the A-d to prevent penetration by environmental water and ototoxic compounds.

In certain embodiments, the kit includes the A-t, the B-d, the C-t, and the D-t. The B-d is administered, wherein the B-d promotes elution of the C-t from the conduit, the C-t is triggered to enable release of the A-t from the conduit, and the D-t is eluted or released subsequent to transport of the A-t to prevent penetration by environmental water and ototoxic compounds or to reverse one or more properties of the conduit to the first configuration.

In certain embodiments, the kit includes the A-p, the C-d, and the D-d. The C-d, and the D-d are administered in a sequential order, wherein the C-d activates release of the A-p from the plug, and the D-d is applied to reverse one or more properties of the conduit to the first configuration.

In certain embodiments, the kit includes the A-p, the B-d, the C-d and the D-d. The B-d, the C-d, and the D-d are administered in a sequential order, wherein the B-d enables transport of C-d, the C-d enables release, elution or application of the A-p from the plug, and the D-d is applied to reverse one or more properties of the conduit to the first configuration.

In certain embodiments, the kit includes the A-d, the B-p, the C-p and the D-t. The A-d is administered, wherein activation of the C-p causes release, elution or application of the B-p from the plug, the B-p that is released, eluted or applied promotes the transport of the A-d, and the D-t is released to reverse one or more properties of the conduit to the first configuration.

In certain embodiments, the kit includes the A-d, the B-d, the B-p, the C-p and the D-t. The A-d, and the B-d are administered in a sequential order, wherein activation of the C-p causes release, elution or application of the B-p from the plug, the B-p that is released, eluted or applied with administration of B-d promote the transport of the A-d, and the D-t is eluted or released to reverse one or more properties of the conduit to the first configuration.

In certain embodiments, the kit includes the A-p, the C-d, and the D-p. The C-d is administered, wherein the C-d activates release, elution or application of the A-p from the plug, and the D-p is released, eluted or applied to reverse one or more properties of the conduit to the first configuration.

In certain embodiments, the kit includes the A-p, the B-d, the C-d and the D-p. The B-d, the C-d are administered in a sequential order, wherein the B-d enables transport of C-d, the C-d enables release, elution or application of the A-p from the plug, and the D-p is released, eluted or applied to reverse one or more properties of the conduit to the first configuration.

In certain embodiments, the kit includes the A-d, the B-p, the C-p and the D-p. The A-d is administered, wherein activation of the C-p causes release, elution or application of the B-p from the plug, the B-p that is released, eluted or applied promotes the transport of the A-d, and the D-p is released, eluted or applied to reverse one or more properties of the conduit to the first configuration.

In certain embodiments, the kit includes the A-d, the B-d, the B-p, the C-p and the D-p. The A-d, and the B-d are administered in a sequential order, wherein activation of the C-p causes release, elution or application of the B-p from the plug, the B-p that is released, eluted or applied with administration of B-d promotes the transport of the A-d, and the D-p is released, eluted or applied subsequent to transport of the A-d to reverse one or more properties of the conduit to the first configuration.

In certain embodiments, one or more of the therapeutic agent A-d, the priming agent B-d, the activating agent C-d, and the reversal agent D-d replenishes one or more of the therapeutic agent A-t, the priming agent B-t, the activating agent C-t, and the reversal agent D-t.

In certain embodiments, one or more of the reversal agent D-d, the reversal agent D-t, and the reversal agent D-p is configured to dissolve, extract or reduce the amount of the lubricating fluid from a part of the inner surface of the conduit.

In certain embodiments, one or more of the priming agent B-d, the priming agent B-t, the priming agent B-p, the activating agent C-d, the activating agent C-t, and the activating agent C-p causes an exothermic reaction, the exothermic reaction modifies a surface tension between the drug formulation and the conduit.

In certain embodiments, one or more of the reversal agent D-d, the reversal agent D-t, and the reversal agent D-p causes an endothermic or exothermic reaction, the endothermic or exothermic reaction causes one of a transitory modification or a permanent modification of a surface tension between the drug formulation and the conduit.

In certain embodiments, the priming agent B-t further includes one or more of a crosslinking shape memory polymer compound, a gelating shape memory polymer compound, and a crosslinking agent.

In certain embodiments, one or more of the D-d, the D-t, and the D-p is a colloidal suspension.

In certain embodiments, one or more of the therapeutic agent A-d, the therapeutic agent A-t, the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p causes a phase transformation of the plug. The phase transformation of the plug enables a transport of one or more of the therapeutic agent A-d, the therapeutic agent A-t, and the therapeutic agent A-p.

In certain embodiments, the drug formulation further includes a solid agent and a liquid agent, wherein the liquid agent is capable of precipitating the solid agent from the drug formulation.

In certain embodiments, the drug formulation further includes a solid agent and a liquid agent, wherein the liquid agent is capable of dissolving the solid agent.

In certain embodiments, the drug formulation further includes a solid agent and a liquid agent. Further, the liquid agent modifies a physicochemical property of the solid agent.

In certain embodiments, the drug formulation is capable of modifying a frictional drag force between one or more of the therapeutic agent A-d, the therapeutic agent A-t, and the therapeutic agent A-p, and the conduit surface from a first value to a second value.

In certain embodiments, the reversal agent is capable of reversing the frictional drag force between one or more of the therapeutic agent A-d, the therapeutic agent A-t, and the therapeutic agent A-p and the conduit surface from the second value to the first value.

In certain embodiments, the drug formulation includes a shear thinning fluid.

In certain embodiments, the shape of the conduit is selected from the group consisting of a tube, a shunt, a stent, a block, a valve, a tab, a wire, a shell, a wick, a channel, a cable, a hose, a pipe, a catheter, a spiral, a helix, a cone, an oval, a duplex shape having two separate concentric transport channels from the proximal and distal ends, a triplex shape having three separate concentric transport channels from the proximal and distal ends, and a combination thereof.

In certain embodiments, a material of the conduit is selected from the group consisting of non-biodegradable compounds, biodegradable compounds, polyurethane, polyurethane copolymer, fluoropolymer, polyolefin, silicone rubber, hydrophobic polyanhydrides, polyorthoesters, polyphosphazenes, polyphosphoesters/phosphoesters, pseudopolyamino acids, poly(ethylene oxide), PEO, and poly(butylene terephthalate), PBT, biodegradable polymers with hydrolysable chemical bonds, wetting agents, emulsifiers, lubricants, sodium lauryl sulfate, calcium, and magnesium stearate, coloring agents, release agents, coating agents, sweetening, flavoring and perfuming agents, preservatives and antioxidants, poloxamers, polyamides, polyimides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene, polyvinylpryrrolidone, alginate, poly(caprolactone), dextran and chitosan, poly(lactic-co-glycolic acid) or PLGA, polycaprolactone or PLC, Gelatin, DNA hydrogen, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and a combination thereof.

In certain embodiments, a material of the conduit is selected from the group consisting of biodegradable polymers, poly(ethylene vinyl acetate), poly(vinyl acetate), silicone polymers, polyurethanes, polyamides, polyimides, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, copolymers, metals, cobalt, chromium, gold, nickel, platinum, stainless steel, titanium, tantalum, nickel-titanium, alloys, and a combination thereof.

In certain embodiments, a material of the conduit is selected from the group consisting of hydrogels, polyvinyl alcohol, polyvinyl pyrrolidone, polyethylene oxide, polyhydroxyethyl methacrylate, hydroxypropylmethyl cellulose and hydroxyethyl cellulose, polyesteramides, polyglycolic acid, polyvinyl alcohol, copolymers of polyethylene oxide/polylactic acid, copolymers of glycolide/lactide, poly-L-lactic acid compounds, polycarbophil, polyvinyl pyrrolidone, polyvinyl alcohol, hydroxyethyl cellulose, hydroxypropylmethyl cellulose, and a combination thereof.

In certain embodiments, a material of the conduit is selected from the group consisting of gels and polymers with stimuli-responsive functionality, thermoresponsive gels, PNIPAAm and its copolymers with hydrophobic or hydrophilic comonomers, copolymer of n-isopropylacrylamide and at least one acrylic and/or methacrylic monomer such as an alkyl acrylate, methyl acrylate, ethyl acrylate, butyl acrylate or 2-ethylhexyl acrylate, an acrylamide, acrylic acid or salt, 2-ethylacrylic acid, 2-propylacrylic acid, N-acryloxysuccinimide, methacrylic monomers, methacrylate, 2-hydroxymethacrylate, hydroxyethyl methacrylate, butyl methacrylate, methyl ether methacrylate, methyl methacrylate, methacrylamide, methacrylic acid and salt, acrylate monomer modified with poly(ethylene glycol), methacrylate monomer modified with poly(ethylene glycol), and a combination thereof.

In certain embodiments, a material of the conduit is selected from the group consisting of biostable or bioabsorbable polymers, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyimides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, chitins, and a combination thereof.

In certain embodiments, a material of the conduit is selected from the group consisting of non-polymers, sterols, cholesterol, stigmasterol, β-sitosterol, and estradiol; cholesteryl esters such as cholesteryl stearate, C12-C24 fatty acids, lauric acid, myristic acid, palmitic acid, stearic acid, arachidic acid, behenic acid, and lignoceric acid, C18-C36 mono-, di- and triacylglycerides, glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate, glyceryl monostearate, acetylated monoglycerides, sucrose fatty acid esters, sucrose distearate and sucrose palmitate, sorbitan fatty acid esters such as sorbitan monostearate, sorbitan monopalmitate, sorbitan tristearate, C16-C18 fatty alcohols, cetyl alcohol, myristyl alcohol, stearyl alcohol, cetostearyl alcohol, esters of fatty alcohols and fatty acids, cetyl palmitate, cetearyl palmitate, anhydrides of fatty acids, stearic anhydride, phospholipids including phosphatidylcholine (lecithin), phosphatidylserine, phosphatidylethanolamine, phosphatidylinositol, and lysoderivatives thereof, sphingosine and derivatives thereof, sphingomyelins, stearyl, palmitoyl, tricosanyl sphingomyelins, ceramides, stearyl, palmitoyl ceramides, glycosphingolipids, lanolin, lanolin alcohols, and a combination thereof.

In certain embodiments, a material of the conduit is selected from the group consisting of silicones, fluoroplastics, Nylon, polyethylene terephthalate, Polycarbonate, Acrylonitrile Butadiene Styrene, Poly(p-phenylene oxide), Polybutylene terephthalate, Acetal, Polypropylene, Polyurethane, Polyetheretherketone, hydroxylpatite, Ultra-high molecular weight polyethylene, High Density Polyethylene, Low Density Polyethylene, Polystyrene High Impact, Polysulfone, Polyvinylidene fluoride, polystyrene, polymethylmethacrylate, latex, polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, polyelectrolyte hydrogels, hydrogel-forming materials, acrylate, polyacrylate, methacrylic acid, (dimethylamino)ethyl methacrylate, hydroxyethyl methacrylate, poly(vinyl alcohol)/poly(acrylic acid), 2-acrylamido-2-methylpropane sulfonic acid, [(methacrylamido)-propyl]trimethyl ammonium chloride, poly(N-vinyl-2-pyrrolidone/itaconic acid), nonionic hydrogels, poly(ethylene glycol), ethylene glycol diacrylate, polyethylene glycol diacrylate poly(ethylene oxide), diacrylate, acrylamide, polyacrylamide, methylenebisacrylamide, N-isopropylacrylamides, poly(vinyl alcohol), hydrogels made of natural materials, hydrogels made of proteins collagen and silk) and polysaccharides (e.g., chitosan, dextran and alginate), superparamagnetic iron oxide nanoparticles (SPION), cubosomes, polymerosomes, and combinations thereof. In some embodiments, the conduits can be made of metals or metal oxides.

In certain embodiments, a material of the conduit is selected from a group consisting of metals, Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Pb, Bi, La, Ce, Pr, Nd, Sm, Eu, Gd, Tb, Dy, Ho, Er, Tm, Yb, Lu, and their oxides, and a combination thereof.

In certain embodiments, a material of the conduit is selected from a group consisting of metal foams, porous metallic substrates, and a combination thereof.

In certain embodiments, a material of the conduit includes colloidal particles. The colloidal particles are selected from a group consisting of polymethylmethacrylate, melamine resins, silica, polydivinylbenzene microparticles, poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, halogenated polymers, hydrogels, organogels, random and block copolymers, branched, star and dendritic polymers, supramolecular polymers, biopolymer colloid, protein- or polysaccharide-based material, silk fibroin, chitin, shellac, cellulose, chitosan, alginate, gelatin, metals, gold, palladium, platinum, silver, copper, rhodium, ruthenium, rhenium, titanium, osmium, iridium, iron, cobalt, or nickel, oxides, silica, alumina, beryllia, noble metal oxides, platinum group metal oxides, titania, tin oxide, zirconia, hafnia, molybdenum oxide, tungsten oxide, rhenium oxide, vanadium oxide, tantalum oxide, niobium oxide, chromium oxide, scandium oxide, yttria, lanthanum oxide, ceria, thorium oxide, uranium oxide, other rare earth oxides, ferromagnetic particles, ferrimagnetic particles, superparamagnetic particles, iron nanoparticles, nickel nanoparticles, cobalt nanoparticles, charged particles, uncharged particles, hydrophilic particles, hydrophobic particles, amphiphilic particles, and a combination thereof.

In certain embodiments, a precursor material is added to the composition of the conduit. The precursor material is selected from the group consisting of small molecules, dispersed liquid droplets, microparticle and nanoparticle fillers, talc, calcium carbonate, calcium phosphate, anti-oxidants, UV stabilizers, plasticizers, anti-static agents, porogens, slip agents, processing aids, foaming or antifoaming agents, nucleating agents and fillers, and a combination thereof.

In certain embodiments, the shape of the plug is selected from the group consisting of a valve, a membrane, a film, a lid, a handle, an inclusion, a pellet, a cage, a capsule, a powder, a foam, a gel, and a combination thereof.

In certain embodiments, a material of the plug is selected from the group consisting of stimuli-responsive polymer, a gas-selective mobile membrane, stimuli-responsive cilia-like and hair-like fibers, platelets, pillars, reconfigurable tunable nano- or microstructures with functionalized tips or functionalized pillars, and combinations thereof.

In certain embodiments, a material of the plug is selected from the group consisting of poly(lactic-co-glycolic acid) or PLGA, polycaprolactone or PLC, Chitosan, Gelatin, DNA hydrogen, acetalated dextran, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polycarbonate, polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and combinations thereof.

In certain embodiments, a material of the plug is selected from the group consisting of commercially available salts containing anions such as acetate, iodide, benzenesulfonate, isethionate, benzoate, lactate, bicarbonate, lactobionate, bitartrate, malate, bromide, maleate, calcium edetate, Mandelate, Carbonate, Methylbromide, Chloride, Methylnitrate, Dihydrochloride, Mucate, Edetate, Napsylate, Edisylate, Nitrate, Esylate, Pantothenate, Fumarate, Phosphateldiphosphate, Gluceptate, Polygalacturonate, Gluconate, Salicylate, Glutamate, Stearate, Glycollylarsanilateg, Subacetate, Hexylresorcinate, Succinate Hydrabamine, Sulfate, Hydrobromide, Tannate, Hydroxynaphthoate, Teoclate, Triethiodide, or cationis such as Benzathid, Aluminum, chloroprocaine, Calcium, Choline, Lithium, Diethanolamine, Magnesium, Ethylenediamine, Potassium, Meglumine, Sodium, Procaine, Zinc, and combination thereof.

In certain embodiments, the stimuli-responsive polymer of the plug is selected from a group consisting of nematic, smectic, chiral, discotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

In certain embodiments, the plug includes an extension, wherein the extension is in form of one or more of a sponge, absorbing pad, foam, receptacle, mesh, porous cladding, inflatable balloons, catheter, fiber, sieve, uniformly or non-uniformly expandable gel, roll, swirl, ball, wrap, film, and dressing.

In certain embodiments, one or more of the conduit and the plug is a microporous absorbent media, wherein the absorbent media is configured to guide and release one or more of the therapeutic agent A-d, the therapeutic agent A-t, and the therapeutic agent A-p.

In certain embodiments, a material of the absorbent media is selected from a group consisting of biocompatible polymers, bioerodable materials, elastomers, metals, metal alloys, glasses, laminates of hydrophilic polymers and hydrophobic polymers, multilaminates or polymer, metals, and/or glasses, biocompatible polymeric materials, homopolymers and copolymers of vinyl acetate, ethylene vinyl acetate copolymer; homopolymers and copolymers of acrylates, poly(methyl) methacrylate (PMMA), polyethylmethacrylate, ethylene glycol dimethacrylate, ethylene dimethacrylate and hydroxymethyl methacrylate, polyurethanes; polyethylenes; polyvinylchlorides; polycarbonates; polyamides; polysulfones; polyesters; polyimides; halogenated polymers, polytetrafluoroethylene (PTFE), polyvinyl fluoride, polychlorotrifluoroethylene, copolymers tetrafiuoroethylene and hexafluoropropylene, PFA, polyolefins, high density polyethylene (HDPE), low density polyethylene (LDPE), linear low density polyethylene (LLDPE), polypropylenes, polystyrenes, nylons; urethanes; homopolymers and copolymers of acrylonitrile, acrylonitrile-butadiene-styrene polymer, styrene acrylonitrile, polycarbonate-acrylonitrile-butadiene-styrene, polyvinylpyrrolidone; 2-pyrrolidone; polyacrylonitrile butadiene, cellulose acetate; polyethylene terephthalate; polymethylpentene; polyisobutylene, polymethylstyrene, polyvinylidine chloride and homopolymers and copolymers of polyvinylidine chloride, polyvinylchloride-acrylic copolymers; PEBAX™; HYTREL™, biocompatible elastomers, silicone rubbers, hydrogels, polyvinyl chloride elastomers; polyolefins, homopolymeric and copolymeric elastomers, urethane-based elastomers; natural rubbers; and fluorinated polymers, PTFE, metallic materials, titanium, platinum, tantalum, gold and their alloys, gold-plated ferrous alloys, platinum-plated titanium, stainless steel, tantalum, gold and their alloys, ferrous alloys, cobalt-chromium alloys, titanium nitride-coated stainless steel, titanium, platinum, tantalum, gold, and their alloys, TEFLON™, nickel titanium, superelastic nickel titanium, and combination thereof.

In certain embodiments, the absorbent media is configured to enhance the flow rate of the transport of one or more of the therapeutic agent A-d, the therapeutic agent A-t, and the therapeutic agent A-p.

In certain embodiments, one or more of the conduit or the plug includes one or more of a chemical sensor, a magnetic sensor, an electrical sensor, and an optical sensor.

In certain embodiments, one or more of the chemical sensor, the magnetic sensor, the electrical sensor, and the optical sensor measures the concentration of one or more of therapeutic agent A-d, the therapeutic agent A-t, and the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p.

In certain embodiments, the conduit is configured to be positioned in an ear, the conduit including: a proximal end configured to be received in an ear canal, the proximal end having a proximal end radius, a distal end opposite the proximal end, the distal end configured to be received in a middle ear, the distal end having a distal end radius, an inner surface connecting the proximal end and the distal end, at least a portion of the inner surface including a conical or curved geometry extending at least partially between the proximal end and the distal end, and an outer surface connecting the proximal end and the distal end; the distal end radius is larger than the proximal end radius. In certain embodiments, the conduit may be applied to other parts of human body for treating other diseases as well, such as for catheters, shunts, stents, tubes, and other conduit-like implantable and non-implantable medical devices and combination products. Non-limiting examples include inner ear conduits, prostatic and biliary stents, naso- and oro-intestinal catheters, sinus cavities, stents for sinus cavities, abdominally-based drains, such as drainage of gallbladder, pancreas, intestine; ventricular catheters, intra-abdominal shunt catheters, glaucoma shunts, nasolacrimal stents, central venous catheters; urinary catheters, vascular catheters, and other conduits.

In certain embodiments, the conduit is configured to have the distal and proximal ends of the same radius with a cylindrical inner surface connecting the ends. In such a conduit, there may be a plurality of holes, cavities, or indentations placed at the distal end or proximal end for transport in various patterns (staggered or parallel) relative to each other, wherein the holes, or cavities, or indentations, themselves are engineered to be curved or conical according to the design principles mentioned in this disclosure.

Certain embodiments of the present disclosure describe a kit for delivering a therapeutic agent to treat disorders. In some embodiments, the kit is configured to deliver therapeutic agent to treat ear disorders. The kit includes two components: a conduit and a therapeutic formulation preloaded into the conduit. The therapeutic formulation includes one or more of a therapeutic agent A-t, a priming agent B-t, an activating agent C-t, and a reversal agent D-t. The priming agent B-t modifies one or more properties of the conduit from a first configuration to a second configuration either upon activation of the priming agent B-t or passively by wetting and/or infusion of the priming agent B-t. The activating agent C-t is capable of releasing the therapeutic agent A-t toward a portion of an inner surface of the conduit upon activation of the activating agent C-t. The reversal agent D-t reverses one or more properties of the conduit from the second configuration to the first configuration upon secretion or activation of the reversal agent D-t.

In certain embodiments, the one or more of the properties include: physical properties, chemical properties, mechanical properties, optical properties, thermal properties, acoustical properties, electrical properties, magnetic properties, geometry, or combinations thereof.

In certain embodiments, the physical properties include wettability, drag, physical heterogeneity, surface energy, surface lubricity, porosity, texture, permeability, swellability, anisotropy, acoustic properties, or combinations thereof; the chemical properties include type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties include Young's moduli, elasticity, stiffness, hardness, toughness, friction, rigidity, compressibility, sound absorption, or combinations thereof; the optical properties include light absorption, light scattering, transmission, or combinations thereof; the thermal properties include glass transition temperature, thermal conductivity, thermal expansion, or combinations thereof; the acoustical properties include acoustical absorption, speed of sound, sound reflection, sound transfer, elasticity, or combinations thereof, for purposes of fluid transport driven by Surface Acoustic Waves (SAW); the electrical properties include capacitance, dielectric constant, resistivity, conductivity, susceptibility, permittivity, piezoelectric constant, or combinations thereof, for electrically driven transport; the magnetic properties include Curie temperature, diamagnetism, hall coefficient, hysteresis, magnetoresistance, permeability, piezomagnetism, spin hall effect, or combinations thereof, for magnetically driven flow; and the geometry includes size, shape, orifice geometry, profile, curvature, or combinations thereof.

In certain embodiments, the kit further includes a drug formulation. The drug formulation includes one or more of a therapeutic agent A-d, a priming agent B-d, an activating agent C-d, and a reversal agent D-d.

In certain embodiments, the therapeutic agent A-d and/or the therapeutic agent A-t is selected from the group consisting of anti-inflammatory, anti-allergic, and analgesic agents, non-steroidal anti-inflammatory (NSAID) agents, tranquilizing agents, corticosteroids, volume expanding agents, vasodilating agents, antihistaminic agents, anticholinergic agents, vaccines, adjuvants, enzymes, monoclonal antibodies, drug dissolution and disintegration promoters, exosomes, microcrystals, solid lipid nanoparticles, preservatives, antifoams, taste-masking agents, chelating agents, buffering agents, bulking agents, anti-coagulants, antiviral agents, immunosuppressive agents, antacids and H2-blockers, antiemetics, calcium channel blockers, anticancer agents, vitamins, silk, vascular rheologic agents, neuroprotective agents, neuromodulators; anti-apoptotic agents; antiseptics, therapeutic peptides, therapeutic polysaccharides, hormones, free radical scavengers, neurotrophins, fungicides, antibodies, antigens, bacteriocides, urea, mannitol, sorbitol, glycerol, lidocaine, xylocaine, epinephrine, immunoglobulins, sodium chloride, heparin, hyaluronidase, aminoglycoside neurotrophins, therapeutic peptides, therapeutic polysaccharides, cortico-steroids, vitamins, cortisone, analgesics, antipyrine, benzocaine, procaine, antioxidants, methionine, N-acetylcysteine, trolox, neurotrophins, GDNF, BDNF, anti-apoptotic agents, anti-necrotic agents, leupeptin or combinations thereof, fentanyl citrate; salicylates; droperidol, prochlorperazine; flucinolone, dexamethasone, prednisone; batahistine, niacin and papaverine; meclizine, dimenhydrinate, scopolamene, and promethazine; glycopyrrolate, propantheline, and atropine; ofloxacin, ciprofloxacin, finafloxacin ampicillin, cefuroxime, amoxicillin and ceftriaxone; cyclophosphomide and cyclosporine; thiazide, triamterene and carbonic anhydrase inhibitors; nizatidine and cimetidine; metoclopramide or diphenidol; diltiazem, nifedipine and verapamil; gentian violet, cersylate, mercurochrome, chloramphenicol, polymyxin; tolnaftate, nystatin, clotrimazole, miconazole; arylcycloalkyamine, ketamine, antibiotics, neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, oat extract (avenanthramide), and combinations thereof.

In certain embodiments, the said therapeutic agent A-d and/or the therapeutic agent A-t is selected from the group consisting of neomycin B, kanamycin A, kanamycin B, erythromycin and azithromycin, vancomycin, gentamicin, amikacin), dibekacin, tobramycin, streptomycin, paromomycin, sisomicin, butirosin, geneticin, isepamicin, and netilmicin, gentamicins A, C1, C1, C2 and D; neomycins B and C, hybrimycin, propikacin (UK 31214), ribostamycin, seldomycin, trehalosamine, D-mannosyl-D-glucosaminide, apramycin, bluensomycin, netromycin, tobramycin, sisomicin, destomycin, Antibiotic A-396-I, dibekacin, kasugamycin, fortimicin, or derivatives; calcineurin inhibitors, interleukin Inhibitors, TNF-α inhibitors, platelet activating factor antagonists, nitric oxide synthase inhibitors, histamine antagonists, estrogen-related receptor beta modulators and combinations thereof.

In certain embodiments, the therapeutic agent A-d and/or the therapeutic agent A-t is selected from the group consisting of thiazide, triamterene, or carbonic anhydrase; diazepam, meclizine, dimenhydrinate, prochlorperazine, promethazine, and preazepam; glycopyrrolate, propantheline, atropine, scopolamine, and combinations thereof.

In certain embodiments, the therapeutic agent A-d and/or the therapeutic agent A-t is selected from the group consisting of probiotics L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, and S. thermophiles, and combinations thereof.

In certain embodiments, the priming agent B-d and/or the priming agent B-t are selected from the group consisting of carrier liquid for the therapeutic agent, surfactants, low-surface-energy liquids, lubricating agents, acids and bases, emulsifiers, rheology control agents, flow-promoting agents, extenders, defoaming agents, plasticizers, thickeners, heat stabilizers, porogens, levelling agents, anti-cratering agents, fillers, UV absorbers, curing agents, diluents, adjuvants, buffering agents, moistening agents, anti-oxidants, oxidizing or reducing agents, mono-dentate or multi-dentate or chelating ligands, crosslinking agents, solubilizing agents, organic and inorganic solvents, SPION, cubosomes, polymersomes, preservatives, polyglycols, long chain hydroxy polyanionic polysaccharides, long chain nitrogen containing polymers, long chain hydroxy polyanionic polysaccharides, glycerol esters, glycerol ethers, glycols, glycolesters and glycolethers, glycerol, monoacetin, diacetin and diacetone alcohol, poly(lactams), polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, waxes, organic co-solvents, alkylene glycols, ethoxylated or propoxylated alcohols, amines, waterless colloid silica, povidone, talc, monolein, magnesium stearate, flavouring agents, lactose, glucose, sucrose, starches, cellulose, derivatives of cellulose, tragacanth, malt, talc, partially or fully fluorinated liquids, oils, cocoa butter, animal fat, vegetable fats, paraffins, silicones, bentonites, silicic acid, zinc oxide, glycols, polyols, esters, agar, buffering agents, alginic acid, pyrogen-free water, isotonic saline, Ringer's solution, ethyl alcohol, phosphate buffer solutions, acacia, colloidal silicon dioxide, calcium glycerophosphate, calcium lactate, maltodextrin, glycerine, magnesium silicate, cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, propylene glycol, dimethyl isosorbide, polyoxyethylene fatty acid glycerides, vegetable oils, polyoxyethylene alkylethers, alkylphenyl ethers, and combinations thereof.

In certain embodiments, the activating agent C-d and/or the activating agent C-t are selected from the group consisting of accelerators, acid scavengers, activators, adhesion promoters, antiblocking agents, antifogging agents, antimicrobials, catalysts, coagulants, colorants, curing agents, thermal stabilizers, viscosity control agents, oxidizing or reducing agents, mono-dentate or multi-dentate or chelating ligands, solubilizing agents, organic and inorganic solvents, acids and bases, pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, azobenzenes, diarylethenes, fulgides, overcrowded alkenes, plasmonic, photothermal or magnetic nanoparticles, catalysts, enzymes, agents capable of extracting therapeutic components from the conduit, and combinations thereof.

In certain embodiments, the reversal agent D-d and/or the reversal agent D-t are selected from the group consisting of stoichiometric reagents, chelators, Ringer's solution, saline solution, water, oils, liquid crystal molecules, thermoresponsive molecules, supramolecules, biomolecules, antibodies, antigens, fluorophores, sterols, surfactants, pigments, dyes, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, diarylethenes, fulgides, overcrowded alkenes, plasmonic, photothermal or magnetic nanoparticles, catalysts, enzymes and combinations thereof.

In certain embodiments, the kit further includes a lubricating fluid formed over at least a portion of the inner surface and the outer surface of the conduit.

In certain embodiments, the lubricating fluid is selected from the group consisting of castor oil, silicone oil, fluocinolone acetonide oil, mineral oil, fully or partially fluorinated hydrocarbons, organosilicone compound, silicone elastomer, tertiary perfluoroalkylamines, the tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, fully, partially or non-fluorinated poly- and oligoethers, fully, partially or non-fluorinated poly- and oligoesters, fully, partially or non-fluorinated poly- and oligoisocyanates, lecithin, glycerin, lipid emulsions, peanut oil, cottonseed oil, safflower oil, sesame oil, olive oil, corn oil and soybean oil, almond oil, borage oil, black currant seed oil, castor oil, corn oil, safflower oil, rapeseed oil, coconut oil, palm oil, canola oil, tea tree oil, and combinations thereof.

In certain embodiments, the therapeutic formulation is embedded in the conduit between the inner surface and the outer surface.

In certain embodiments, the kit further includes a plug over the inner surface of the conduit. The plug includes a plug formulation, and the plug formulation includes one or more of a therapeutic agent A-p, a priming agent B-p, an activating agent C-p, and a reversal agent D-p.

In certain embodiments, any one of the priming agent B-d, the priming agent B-t, or the priming agent B-p is the carrier liquid for any one of the therapeutic agent A-d, the therapeutic agent A-t, or the therapeutic agent A-p.

In certain embodiments, one or more of the priming agent B-d, the priming agent B-t, and the priming agent B-p is a low-surface-tension liquid.

In certain embodiments, one or more of the priming agent B-d, the priming agent B-t, and the priming agent B-p is the lubricating fluid.

In certain embodiments, one or more of the priming agent B-d, the priming agent B-t, and the priming agent B-p is silicone oil.

In certain embodiments, one or more of the therapeutic agent A-d, the priming agent B-d, the activating agent C-d, and the reversal agent D-d are administered sequentially.

In certain embodiments, the kit includes the A-d and the D-t. The A-d is deposited onto the conduit, and the D-t is released from the conduit to modify or close the conduit subsequent to transport of the A-d to prevent penetration by environmental water and ototoxic compounds.

In certain embodiments, the kit includes the A-d, the C-d, and the D-t. The A-d, the C-d, and the D-t are administered in a sequential order, wherein the A-d is deposited into the conduit, the C-d stimulates the progression of the A-d through the conduit, and the D-t is applied to modify or close the conduit subsequent to transport of the A-d to prevent penetration by environmental water and ototoxic compounds.

In certain embodiments, the kit includes the A-d, the C-d, and the D-t. The C-d, and the A-d are administered in a sequential order, wherein deposition of the C-d allows penetration of the A-d to be triggered, and the D-t is secreted from the conduit or activated within the conduit to modify or close the conduit subsequent to transport of the A-d to prevent penetration by environmental water and ototoxic compounds.

In certain embodiments, the kit includes the A-d, the B-d, and the D-t. The B-d, and the A-d are administered in a sequential order, wherein the B-d is applied to promote transport of the A-d, and the D-t is released from the conduit to modify or close the conduit subsequent to transport of the A-d to prevent penetration by environmental water and ototoxic compounds.

In certain embodiments, the kit includes the A-d, the B-d, the C-d and the D-t. The B-d, the C-d, and the A-d are administered in a sequential order, wherein the B-d is applied to promote transport of the C-d, the C-d is triggered to allow penetration of the A-d, and the D-t is released from the conduit to modify or close the conduit subsequent to transport of the A-d to prevent penetration by environmental water and ototoxic compounds.

In certain embodiments, the kit includes the A-t, the B-d, and the D-t, wherein the B-d is administered. The B-d allows A-t to desorb from the conduit, and the D-t is released from the conduit to modify or close the conduit subsequent to transport of the A-t to prevent penetration by environmental water and ototoxic compounds.

In certain embodiments, the kit includes the A-t, the C-d, and the D-d. The C-d, and the D-d are administered in a sequential order, wherein the C-d triggers the A-t to be cleaved, decomposed, or eluted from the conduit, and the D-d is applied to modify or close the conduit subsequent to transport of the A-t to prevent penetration by environmental water and ototoxic compounds.

In certain embodiments, the kit includes the A-t, the B-d, the C-d, and the D-d. The B-d, the C-d, and the D-d are administered in a sequential order, wherein the B-d is applied to promote transport of the C-d, the C-d is triggered to enable release of the A-t from the conduit, and the D-d is applied to modify or close the conduit subsequent to transport of the A-t to prevent penetration by environmental water and ototoxic compounds

In certain embodiments, the kit includes the A-t, the B-d, and the C-d. The B-d, and the C-d are administered in a sequential order, wherein the B-d is applied to promote transport of the C-d, the C-d is triggered to enable release of the A-t from the conduit.

In certain embodiments, the kit includes the A-t, the B-d, the C-d, and the D-d. The B-d, the C-d, and the D-d are administered in a sequential order, wherein the B-d activates transport of the C-d, the C-d triggers the release of the A-t from the conduit, and the D-d is applied to modify or close the conduit subsequent to transport of the A-t to prevent penetration by environmental water and ototoxic compounds.

In certain embodiments, the kit includes the A-t, the B-d, the C-d, and the D-d. The C-d, the B-d, and the D-d are administered in a sequential order, wherein the C-d activates transport of the B-d, the B-d triggers the release of the A-t from the conduit, and the D-d is applied to modify or close the conduit subsequent to transport of the A-t to prevent penetration by environmental water and ototoxic compounds.

In certain embodiments, the kit includes the A-d, the B-t, and the C-t. The A-d is administered, wherein the activation of the C-t causes the B-t to elute from the conduit, the B-t that is eluted enables transport of the A-d.

In certain embodiments, the kit includes the A-d, the B-t, the C-d, and the C-t. The A-d, and the C-d are administered in a sequential order, wherein activation of the C-t causes the B-t to desorb or elute from the conduit, the B-t that is desorbed or eluted, in conjunction with a stimulus or activation provided by the C-d, enables transport of the A-d through the conduit.

In certain embodiments, the kit includes the A-d, the B-d, the B-t, and the C-t. The B-d, and the A-d are administered in a sequential order, wherein the B-d is administered in the lumen, the B-t and the C-t are released from the conduit by an external stimulus, facilitating transport of the A-d.

In certain embodiments, the kit further includes D-d, wherein the D-d is administered after the C-d, wherein the D-d is applied to modify or close the conduit subsequent to transport of the A-d to prevent penetration by environmental water and ototoxic compounds.

In certain embodiments, the kit includes the A-t, the B-d, the C-t, and the D-t. The B-d, and the C-d are administered in a sequential order, wherein the B-d promotes elution of the C-t from the conduit, the C-d is triggered to enable release of the A-t from the conduit, and the D-t is released to reverse one or more properties of the conduit to the first configuration.

In certain embodiments, one or more of the reversal agent D-d, the reversal agent D-t, and the reversal agent D-p is configured to dissolve the lubricating fluid from a part of the inner surface of the conduit.

In certain embodiments, one or more of the priming agent B-d, the priming agent B-t, the priming agent B-p, the activating agent C-d, the activating agent C-t, and the activating agent C-p causes an exothermic reaction, the exothermic reaction modifies a surface tension between the therapeutic agent and the conduit.

In certain embodiments, one or more of the reversal agent D-d, the reversal agent D-t, and the reversal agent D-p causes an endothermic or exothermic reaction, the endothermic or exothermic reaction causes one of a transitory modification or a permanent modification of a surface tension between the therapeutic agent and the conduit.

In certain embodiments, the priming agent B-t further includes one or more of a crosslinking shape memory polymer compound, a gelating shape memory polymer compound, and a crosslinking agent.

In certain embodiments, one or more of the reversal agent D-d, the priming agent D-t, and the priming agent D-p is a colloidal suspension. The colloidal suspension is adsorbed on the inner surface of the conduit to enable one of a dewetting or a pinning of any of the therapeutic agent A-d, the therapeutic agent A-t, or the therapeutic agent A-p.

In certain embodiments, one or more of the therapeutic agent A-d, the therapeutic agent A-t, the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p causes a phase transformation of the plug. The phase transformation of the plug enables a transport of one or more of the therapeutic agent A-d, the therapeutic agent A-t, and the therapeutic agent A-p.

In certain embodiments, the drug formulation further comprises a solid agent and a liquid agent. The liquid agent is capable of precipitating out the solid agent from the drug formulation.

In certain embodiments, the drug formulation further comprises a solid agent and a liquid agent. The liquid agent is capable of dissolving the solid agent.

In certain embodiments, the drug formulation further comprises a solid agent and a liquid agent. The liquid agent modifies a physicochemical property of the solid agent.

In certain embodiments, one or more of the drug formulation and the therapeutic formulation is capable of modifying a frictional drag force between one or more of the therapeutic agent A-d, the therapeutic agent A-t, and the therapeutic agent A-p, and the conduit surface from a first value to a second value.

In certain embodiments, the reversal agent is capable of reversing the frictional drag force between one or more of the therapeutic agent A-d, the therapeutic agent A-t, and the therapeutic agent A-p and the conduit surface from the second value to the first value.

In certain embodiments, one or more of the drug formulation, the therapeutic formulation and the plug formulation includes a shear thinning fluid.

In certain embodiments, the shape of the conduit is selected from the group consisting of a tube, a shunt, a stent, a block, a valve, a tab, a wire, a shell, a wick, a channel, a cable, a hose, a pipe, a catheter, a spiral, a helix, a cone, an oval, a duplex shape having two separate concentric transport channels from the proximal and distal ends, a triplex shape having three separate concentric transport channels from the proximal and distal ends, and a combination thereof.

In certain embodiments, a material of the conduit is selected from the group consisting of non-biodegradable compounds, biodegradable compounds, polyurethane, polyurethane copolymer, fluoropolymer, polyolefin, silicone rubber, hydrophobic polyanhydrides, polyorthoesters, polyphosphazenes, polyphosphoesters/phosphoesters, pseudopolyamino acids, poly(ethylene oxide), PEO, and poly(butylene terephthalate), PBT, biodegradable polymers with hydrolysable chemical bonds, wetting agents, emulsifiers, lubricants, sodium lauryl sulfate, calcium, and magnesium stearate, coloring agents, release agents, coating agents, sweetening, flavoring and perfuming agents, preservatives and antioxidants, poloxamers, polyamides, polyimides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene, polyvinylpryrrolidone, alginate, poly(caprolactone), dextran and chitosan, poly(lactic-co-glycolic acid) or PLGA, polycaprolactone or PLC, Gelatin, DNA hydrogen, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and a combination thereof.

In certain embodiments, a material of the conduit is selected from the group consisting of biodegradable polymers, poly(ethylene vinyl acetate), poly(vinyl acetate), silicone polymers, polyurethanes, polyamides, polyimides, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, copolymers, metals, cobalt, chromium, gold, nickel, platinum, stainless steel, titanium, tantalum, nickel-titanium, alloys, and a combination thereof.

In certain embodiments, a material of the conduit is selected from the group consisting of hydrogels, polyvinyl alcohol, polyvinyl pyrrolidone, polyethylene oxide, polyhydroxyethyl methacrylate, hydroxypropylmethyl cellulose and hydroxyethyl cellulose, polyesteramides, polyglycolic acid, polyvinyl alcohol, copolymers of polyethylene oxide/polylactic acid, copolymers of glycolide/lactide, poly-L-lactic acid compounds, polycarbophil, polyvinyl pyrrolidone, polyvinyl alcohol, hydroxyethyl cellulose, hydroxypropylmethyl cellulose, and a combination thereof.

In certain embodiments, a material of the conduit is selected from the group consisting of gels and polymers with stimuli-responsive functionality, thermoresponsive gels, PNIPAAm and its copolymers with hydrophobic or hydrophilic comonomers, copolymer of n-isopropylacrylamide and at least one acrylic and/or methacrylic monomer such as an alkyl acrylate, methyl acrylate, ethyl acrylate, butyl acrylate or 2-ethylhexyl acrylate, an acrylamide, acrylic acid or salt, 2-ethylacrylic acid, 2-propylacrylic acid, N-acryloxysuccinimide, methacrylic monomers, methacrylate, 2-hydroxymethacrylate, hydroxyethyl methacrylate, butyl methacrylate, methyl ether methacrylate, methyl methacrylate, methacrylamide, methacrylic acid and salt, acrylate monomer modified with poly(ethylene glycol), methacrylate monomer modified with poly(ethylene glycol), and a combination thereof.

In certain embodiments, a material of the conduit is selected from the group consisting of biostable or bioabsorbable polymers, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyimides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, chitins, and a combination thereof.

In certain embodiments, a material of the conduit is selected from the group consisting of non-polymers, sterols, cholesterol, stigmasterol, β-sitosterol, and estradiol; cholesteryl esters such as cholesteryl stearate, C12-C24 fatty acids, lauric acid, myristic acid, palmitic acid, stearic acid, arachidic acid, behenic acid, and lignoceric acid, C18-C36 mono-, di- and triacylglycerides, glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate, glyceryl monostearate, acetylated monoglycerides, sucrose fatty acid esters, sucrose distearate and sucrose palmitate, sorbitan fatty acid esters such as sorbitan monostearate, sorbitan monopalmitate, sorbitan tristearate, C16-C18 fatty alcohols, cetyl alcohol, myristyl alcohol, stearyl alcohol, cetostearyl alcohol, esters of fatty alcohols and fatty acids, cetyl palmitate, cetearyl palmitate, anhydrides of fatty acids, stearic anhydride, phospholipids including phosphatidylcholine (lecithin), phosphatidylserine, phosphatidylethanolamine, phosphatidylinositol, and lysoderivatives thereof, sphingosine and derivatives thereof, sphingomyelins, stearyl, palmitoyl, tricosanyl sphingomyelins, ceramides, stearyl, palmitoyl ceramides, glycosphingolipids, lanolin, lanolin alcohols, and a combination thereof.

In certain embodiments, a material of the conduit is selected from the group consisting of silicones, fluoroplastics, Nylon, polyethylene terephthalate, Polycarbonate, Acrylonitrile Butadiene Styrene, Poly(p-phenylene oxide), Polybutylene terephthalate, Acetal, Polypropylene, Polyurethane, Polyetheretherketone, hydroxylpatite, Ultra-high molecular weight polyethylene, High Density Polyethylene, Low Density Polyethylene, Polystyrene High Impact, Polysulfone, Polyvinylidene fluoride, polystyrene, polymethylmethacrylate, latex, polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, polyelectrolyte hydrogels, hydrogel-forming materials, acrylate, polyacrylate, methacrylic acid, (dimethylamino)ethyl methacrylate, hydroxyethyl methacrylate, poly(vinyl alcohol)/poly(acrylic acid), 2-acrylamido-2-methylpropane sulfonic acid, [(methacrylamido)-propyl]trimethyl ammonium chloride, poly(N-vinyl-2-pyrrolidone/itaconic acid), nonionic hydrogels, poly(ethylene glycol), ethylene glycol diacrylate, polyethylene glycol diacrylate poly(ethylene oxide), diacrylate, acrylamide, polyacrylamide, methylenebisacrylamide, N-isopropylacrylamides, poly(vinyl alcohol), hydrogels made of natural materials, hydrogels made of proteins collagen and silk) and polysaccharides (e.g., chitosan, dextran and alginate), superparamagnetic iron oxide nanoparticles (SPION), cubosomes, polymerosomes, and combinations thereof. In some embodiments, the conduits can be made of metals or metal oxides.

In certain embodiments, a material of the conduit is selected from a group consisting of metals, Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Ti, Pb, Bi, La, Ce, Pr, Nd, Sm, Eu, Gd, Tb, Dy, Ho, Er, Tm, Yb, Lu, and their oxides, and a combination thereof.

In certain embodiments, a material of the conduit is selected from a group consisting of metal foams, porous metallic substrates, and a combination thereof.

In certain embodiments, a material of the conduit includes colloidal particles. The colloidal particles are selected from a group consisting of polymethylmethacrylate, melamine resins, silica, polydivinylbenzene microparticles, poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, halogenated polymers, hydrogels, organogels, random and block copolymers, branched, star and dendritic polymers, supramolecular polymers, biopolymer colloid, protein- or polysaccharide-based material, silk fibroin, chitin, shellac, cellulose, chitosan, alginate, gelatin, metals, gold, palladium, platinum, silver, copper, rhodium, ruthenium, rhenium, titanium, osmium, iridium, iron, cobalt, or nickel, oxides, silica, alumina, beryllia, noble metal oxides, platinum group metal oxides, titania, tin oxide, zirconia, hafnia, molybdenum oxide, tungsten oxide, rhenium oxide, vanadium oxide, tantalum oxide, niobium oxide, chromium oxide, scandium oxide, yttria, lanthanum oxide, ceria, thorium oxide, uranium oxide, other rare earth oxides, ferromagnetic particles, ferrimagnetic particles, superparamagnetic particles, iron nanoparticles, nickel nanoparticles, cobalt nanoparticles, charged particles, uncharged particles, hydrophilic particles, hydrophobic particles, amphiphilic particles, and a combination thereof.

In certain embodiments, a precursor material is added to the composition of the conduit. The precursor material is selected from the group consisting of small molecules, dispersed liquid droplets, microparticle and nanoparticle fillers, talc, calcium carbonate, calcium phosphate, anti-oxidants, UV stabilizers, plasticizers, anti-static agents, porogens, slip agents, processing aids, foaming or antifoaming agents, nucleating agents and fillers, and a combination thereof.

In certain embodiments, the shape of the plug is selected from the group consisting of a valve, a membrane, a film, a lid, a handle, an inclusion, a pellet, a cage, a capsule, a powder, a foam, a gel, and a combination thereof.

In certain embodiments, a material of the plug is selected from the group consisting of stimuli-responsive polymer, a gas-selective mobile membrane, stimuli-responsive cilia-like and hair-like fibers, platelets, pillars, reconfigurable tunable nano- or microstructures with functionalized tips or functionalized pillars, and combinations thereof.

In certain embodiments, a material of the plug is selected from the group consisting of poly(lactic-co-glycolic acid) or PLGA, polycaprolactone or PLC, Chitosan, Gelatin, DNA hydrogen, acetalated dextran, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polycarbonate, polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and combinations thereof.

In certain embodiments, a material of the plug is selected from the group consisting of commercially available salts containing anions such as acetate, iodide, benzenesulfonate, isethionate, benzoate, lactate, Bbicarbonate, lactobionate, bitartrate, malate, bromide, maleate, calcium edetate, Mandelate, Carbonate, Methylbromide, Chloride, Methylnitrate, Dihydrochloride, Mucate, Edetate, Napsylate, Edisylate, Nitrate, Esylate, Pantothenate, Fumarate, Phosphateldiphosphate, Gluceptate, Polygalacturonate, Gluconate, Salicylate, Glutamate, Stearate, Glycollylarsanilateg, Subacetate, Hexylresorcinate, Succinate Hydrabamine, Sulfate, Hydrobromide, Tannate, Hydroxynaphthoate, Teoclate, Triethiodide, or cations such as Benzathid, Aluminum, chloroprocaine, Calcium, Choline, Lithium, Diethanolamine, Magnesium, Ethylenediamine, Potassium, Meglumine, Sodium, Procaine, Zinc, and combination thereof.

In certain embodiments, the stimuli-responsive polymer of the plug is selected from a group consisting of nematic, smectic, chiral, discotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

In certain embodiments, the plug includes an extension. The extension is in form of one or more of a sponge, absorbing pad, foam, receptacle, mesh, porous cladding, inflatable balloons, catheter, fiber, sieve, uniformly or non-uniformly expandable gel, roll, swirl, ball, wrap, film, and dressing.

In certain embodiments, one or more of the conduit and the plug is a microporous absorbent media. The absorbent media is configured to guide and release one or more of the therapeutic agent A-d, the therapeutic agent A-t, and the therapeutic agent A-p.

In certain embodiments, a material of the absorbent media is selected from a group consisting of biocompatible polymers, bioerodable materials, elastomers, metals, metal alloys, glasses, laminates of hydrophilic polymers and hydrophobic polymers, multilaminates or polymer, metals, and/or glasses, biocompatible polymeric materials, homopolymers and copolymers of vinyl acetate, ethylene vinyl acetate copolymer; homopolymers and copolymers of acrylates, poly(methyl) methacrylate (PMMA), polyethylmethacrylate, ethylene glycol dimethacrylate, ethylene dimethacrylate and hydroxymethyl methacrylate, polyurethanes; polyethylenes; polyvinylchlorides; polycarbonates; polyamides; polysulfones; polyesters; polyimides; halogenated polymers, polytetrafluoroethylene (PTFE), polyvinyl fluoride, polychlorotrifluoroethylene, copolymers tetrafiuoroethylene and hexafluoropropylene, PFA, polyolefins, high density polyethylene (HDPE), low density polyethylene (LDPE), linear low density polyethylene (LLDPE), polypropylenes, polystyrenes, nylons; urethanes; homopolymers and copolymers of acrylonitrile, acrylonitrile-butadiene-styrene polymer, styrene acrylonitrile, polycarbonate-acrylonitrile-butadiene-styrene, polyvinylpyrrolidone; 2-pyrrolidone; polyacrylonitrile butadiene, cellulose acetate; polyethylene terephthalate; polymethylpentene; polyisobutylene, polymethylstyrene, polyvinylidine chloride and homopolymers and copolymers of polyvinylidine chloride, polyvinylchloride-acrylic copolymers; PEBAX™; HYTREL™, biocompatible elastomers, silicone rubbers, hydrogels, polyvinyl chloride elastomers; polyolefins, homopolymeric and copolymeric elastomers, urethane-based elastomers; natural rubbers; and fluorinated polymers, PTFE, metallic materials, titanium, platinum, tantalum, gold and their alloys, gold-plated ferrous alloys, platinum-plated titanium, stainless steel, tantalum, gold and their alloys, ferrous alloys, cobalt-chromium alloys, titanium nitride-coated stainless steel, titanium, platinum, tantalum, gold, and their alloys, TEFLON™, nickel titanium, superelastic nickel titanium, and combination thereof.

In certain embodiments, the absorbent media is configured to enhance the flow rate of the transport of one or more of the therapeutic agent A-d, the therapeutic agent A-t, and the therapeutic agent A-p.

In certain embodiments, one or more of the conduit and the plug includes one or more of a chemical sensor, a magnetic sensor, an electrical sensor, and an optical sensor.

In certain embodiments, one or more of the chemical sensor, the magnetic sensor, the electrical sensor, and the optical sensor measures the concentration of one or more of therapeutic agent A-d, the therapeutic agent A-t, and the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p.

In certain embodiments, the conduit is configured to be positioned in an ear, the conduit including: a proximal end configured to be received in an ear canal, the proximal end having a proximal end radius, a distal end opposite the proximal end, the distal end configured to be received in a middle ear, the distal end having a distal end radius, an inner surface connecting the proximal end and the distal end, at least a portion of the inner surface including a conical or curved geometry extending at least partially between the proximal end and the distal end, and an outer surface connecting the proximal end and the distal end; the distal end radius is larger than the proximal end radius. In certain embodiments, the conduit may be applied to other parts of human body for treating other diseases as well, such as for catheters, shunts, stents, tubes, and other conduit-like implantable and non-implantable medical devices and combination products. Non-limiting examples include inner ear conduits, prostatic and biliary stents, naso- and oro-intestinal catheters, sinus cavities, stents for sinus cavities, abdominally-based drains, such as drainage of gallbladder, pancreas, intestine; ventricular catheters, intra-abdominal shunt catheters, glaucoma shunts, nasolacrimal stents, central venous catheters; urinary catheters, vascular catheters, and other conduits.

Certain embodiments of the present disclosure describe a kit for delivering a therapeutic agent to treat disorders. In some embodiments, the kit is configured to deliver therapeutic agent to treat ear disorders. The kit includes two components: a conduit and a plug for insertion into the conduit. The plug includes a plug formulation comprising one or more of a therapeutic agent A-p, a priming agent B-p, an activating agent C-p, and a reversal agent D-p. The priming agent B-p modifies one or more properties of the conduit from a first configuration to a second configuration either upon activation of the priming agent B-p or passively by wetting and/or infusion of the priming agent B-p. The activating agent C-p is capable of releasing the therapeutic agent A-p toward a portion of an inner surface of the conduit upon activation of the activating agent C-p. The reversal agent D-p reverses one or more properties of the conduit from the second configuration to the first configuration upon activation of the reversal agent D-p.

In certain embodiments, the one or more of the properties include: physical properties, chemical properties, mechanical properties, optical properties, thermal properties, acoustical properties, electrical properties, magnetic properties, geometry, or combinations thereof.

In certain embodiments, the physical properties include wettability, drag, physical heterogeneity, surface energy, surface lubricity, porosity, texture, permeability, swellability, anisotropy, acoustic properties, or combinations thereof; the chemical properties include type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties include Young's moduli, elasticity, stiffness, hardness, toughness, friction, rigidity, compressibility, sound absorption, or combinations thereof; the optical properties include light absorption, light scattering, transmission, or combinations thereof; the thermal properties include glass transition temperature, thermal conductivity, thermal expansion, or combinations thereof; the acoustical properties include acoustical absorption, speed of sound, sound reflection, sound transfer, elasticity, or combinations thereof, for purposes of fluid transport driven by Surface Acoustic Waves (SAW); the electrical properties include capacitance, dielectric constant, resistivity, conductivity, susceptibility, permittivity, piezoelectric constant, or combinations thereof, for electrically driven transport; the magnetic properties include Curie temperature, diamagnetism, hall coefficient, hysteresis, magnetoresistance, permeability, piezomagnetism, spin hall effect, or combinations thereof, for magnetically driven flow; and the geometry includes size, shape, orifice geometry, profile, curvature, or combinations thereof.

In certain embodiments, the kit further includes a drug formulation. The drug formulation includes one or more of a therapeutic agent A-d, a priming agent B-d, an activating agent C-d, and a reversal agent D-d.

In certain embodiments, the therapeutic agent A-d and/or the therapeutic agent A-p is selected from the group consisting of anti-inflammatory, anti-allergic, and analgesic agents, non-steroidal anti-inflammatory (NSAID) agents, tranquilizing agents, corticosteroids, volume expanding agents, vasodilating agents, antihistaminic agents, anticholinergic agents, vaccines, adjuvants, enzymes, monoclonal antibodies, drug dissolution and disintegration promoters, exosomes, microcrystals, solid lipid nanoparticles, preservatives, antifoams, taste-masking agents, chelating agents, buffering agents, bulking agents, anti-coagulants, antiviral agents, immunosuppressive agents, antacids and H2-blockers, antiemetics, calcium channel blockers, anticancer agents, vitamins, silk, vascular rheologic agents, neuroprotective agents, neuromodulators; anti-apoptotic agents; antiseptics, therapeutic peptides, therapeutic polysaccharides, hormones, free radical scavengers, neurotrophins, fungicides, antibodies, antigens, bacteriocides, urea, mannitol, sorbitol, glycerol, lidocaine, xylocaine, epinephrine, immunoglobulins, sodium chloride, heparin, hyaluronidase, aminoglycoside neurotrophins, therapeutic peptides, therapeutic polysaccharides, cortico-steroids, vitamins, cortisone, analgesics, antipyrine, benzocaine, procaine, antioxidants, methionine, N-acetylcysteine, trolox, neurotrophins, GDNF, BDNF, anti-apoptotic agents, anti-necrotic agents, leupeptin or combinations thereof, fentanyl citrate; salicylates; droperidol, prochlorperazine; flucinolone, dexamethasone, prednisone; batahistine, niacin and papaverine; meclizine, dimenhydrinate, scopolamene, and promethazine; glycopyrrolate, propantheline, and atropine; ofloxacin, ciprofloxacin, finafloxacin ampicillin, cefuroxime, amoxicillin and ceftriaxone; cyclophosphomide and cyclosporine; thiazide, triamterene and carbonic anhydrase inhibitors; nizatidine and cimetidine; metoclopramide or diphenidol; diltiazem, nifedipine and verapamil; gentian violet, cersylate, mercurochrome, chloramphenicol, polymyxin; tolnaftate, nystatin, clotrimazole, miconazole; arylcycloalkyamine, ketamine, antibiotics, neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, oat extract (avenanthramide), and combinations thereof.

In certain embodiments, the therapeutic agent A-d and/or the therapeutic agent A-p is selected from the group consisting of neomycin B, kanamycin A, kanamycin B, erythromycin and azithromycin, vancomycin, gentamicin, amikacin), dibekacin, tobramycin, streptomycin, paromomycin, sisomicin, butirosin, geneticin, isepamicin, and netilmicin, gentamicins A, C1, C1, C2 and D; neomycins B and C, hybrimycin, propikacin (UK 31214), ribostamycin, seldomycin, trehalosamine, D-mannosyl-D-glucosaminide, apramycin, bluensomycin, netromycin, tobramycin, sisomicin, destomycin, Antibiotic A-396-I, dibekacin, kasugamycin, fortimicin, or derivatives; calcineurin inhibitors, interleukin Inhibitors, TNF-α inhibitors, platelet activating factor antagonists, nitric oxide synthase inhibitors, histamine antagonists, estrogen-related receptor beta modulators and combinations thereof.

In certain embodiments, the therapeutic agent A-d and/or the therapeutic agent A-p is selected from the group consisting of thiazide, triamterene, or carbonic anhydrase; diazepam, meclizine, dimenhydrinate, prochlorperazine, promethazine, and preazepam; glycopyrrolate, propantheline, atropine, scopolamine, and combinations thereof.

In certain embodiments, the therapeutic agent A-d and/or the therapeutic agent A-t is selected from the group consisting of probiotics L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, and S. thermophiles, and combinations thereof.

In certain embodiments, the priming agent B-d and/or the priming agent B-p are selected from the group consisting of carrier liquid for the therapeutic agent, surfactants, low-surface-energy liquids, lubricating agents, acids and bases, emulsifiers, rheology control agents, flow-promoting agents, extenders, defoaming agents, plasticizers, thickeners, heat stabilizers, porogens, levelling agents, anti-cratering agents, fillers, UV absorbers, curing agents, diluents, adjuvants, buffering agents, moistening agents, anti-oxidants, oxidizing or reducing agents, mono-dentate or multi-dentate or chelating ligands, crosslinking agents, solubilizing agents, organic and inorganic solvents, SPION, cubosomes, polymersomes, preservatives, polyglycols, long chain hydroxy polyanionic polysaccharides, long chain nitrogen containing polymers, long chain hydroxy polyanionic polysaccharides, glycerol esters, glycerol ethers, glycols, glycolesters and glycolethers, glycerol, monoacetin, diacetin and diacetone alcohol, poly(lactams), polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, waxes, organic co-solvents, alkylene glycols, ethoxylated or propoxylated alcohols, amines, waterless colloid silica, povidone, talc, monolein, magnesium stearate, flavouring agents, lactose, glucose, sucrose, starches, cellulose, derivatives of cellulose, tragacanth, malt, talc, partially or fully fluorinated liquids, oils, cocoa butter, animal fat, vegetable fats, paraffins, silicones, bentonites, silicic acid, zinc oxide, glycols, polyols, esters, agar, buffering agents, alginic acid, pyrogen-free water, isotonic saline, Ringer's solution, ethyl alcohol, phosphate buffer solutions, acacia, colloidal silicon dioxide, calcium glycerophosphate, calcium lactate, maltodextrin, glycerol, magnesium silicate, cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, propylene glycol, dimethyl isosorbide, polyoxyethylene fatty acid glycerides, vegetable oils, polyoxyethylene alkylethers, alkylphenyl ethers, and combinations thereof.

In certain embodiments, the activating agent C-d and/or the activating agent C-p are selected from the group consisting of accelerators, acid scavengers, activators, adhesion promoters, antiblocking agents, antifogging agents, antimicrobials, catalysts, coagulants, colorants, curing agents, thermal stabilizers, viscosity control agents, oxidizing or reducing agents, mono-dentate or multi-dentate or chelating ligands, solubilizing agents, organic and inorganic solvents, acids and bases, pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, azobenzenes, diarylethenes, fulgides, overcrowded alkenes, plasmonic, photothermal or magnetic nanoparticles, catalysts, enzymes, agents capable of extracting therapeutic components from the conduit, and combinations thereof.

In certain embodiments, the reversal agent D-d and/or the reversal agent D-p are selected from the group consisting of stoichiometric reagents, chelators, Ringer's solution, saline solution, water, oils, liquid crystal molecules, thermoresponsive molecules, supramolecules, biomolecules, antibodies, antigens, fluorophores, sterols, surfactants, In some embodiments, the reversal agent is selected from the group consisting of pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, diarylethenes, fulgides, overcrowded alkenes, plasmonic, photothermal or magnetic nanoparticles, catalysts, enzymes and combinations thereof.

In certain embodiments, the kit further includes a lubricating fluid formed over at least a portion of the inner surface of the conduit.

In certain embodiments, the lubricating fluid is selected from the group consisting of castor oil, silicone oils, fluocinolone acetonide oil, mineral oil, fully or partially fluorinated hydrocarbons, organosilicone compound, silicones, tertiary perfluoroalkylamines, the tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, fully, partially or non-fluorinated poly- and oligoethers, fully, partially or non-fluorinated poly- and oligoesters, fully, partially or non-fluorinated poly- and oligoisocyanates, lecithin, glycerin, lipid emulsions, peanut oil, cottonseed oil, safflower oil, sesame oil, olive oil, corn oil and soybean oil, almond oil, borage oil, black currant seed oil, castor oil, corn oil, safflower oil, rapeseed oil, coconut oil, palm oil, canola oil, tea tree oil, and combinations thereof.

In certain embodiments, the kit further includes a therapeutic formulation preloaded into the conduit, wherein the therapeutic formulation comprises one or more of a therapeutic agent A-t, a priming agent B-t, an activating agent C-t, and a reversal agent D-t.

In certain embodiments, any one of the priming agent B-d, the priming agent B-t, or the priming agent B-p is the carrier liquid for any one of the therapeutic agent A-d, the therapeutic agent A-t, or the therapeutic agent A-p.

In certain embodiments, one or more of the priming agent B-d, the priming agent B-t, and the priming agent B-p is a low-surface-tension liquid.

In certain embodiments, one or more of the priming agent B-d, the priming agent B-t, and the priming agent B-p is the lubricating fluid.

In certain embodiments, one or more of the priming agent B-d, the priming agent B-t, and the priming agent B-p is silicone oil.

In certain embodiments, one or more of the therapeutic agent A-d, the priming agent B-d, the activating agent C-d, and the reversal agent D-d is administered sequentially.

In certain embodiments, the kit includes the A-p, the C-d, and the D-d. The C-d, and the D-d are administered in a sequential order, wherein the C-d activates the release, elution or application of the A-p from the plug, and the D-d applied to reverse one or more properties of the conduit to the first configuration to prevent the elution of the A-p from the plug.

In certain embodiments, the kit includes the A-p, the B-d, the C-d and the D-d. The B-d, the C-d, and the D-d are administered in a sequential order, wherein the B-d enables transport of C-d, the C-d enables release, elution or application the A-p from the plug, and the D-d is applied to reverse one or more properties of the conduit to the first configuration.

In certain embodiments, the kit includes the A-d, the B-p, the C-p and the D-t. The A-d is administered to activate the C-p, which then will cause release, elution or application of the B-p from the plug, in turn, the B-p that is released, eluted or applied promotes the transport of the A-d, and the D-t is eluted or released to reverse one or more properties of the conduit to the first configuration.

In certain embodiments, the kit includes the A-p, the C-d, and the D-p. The C-d activates release, elution or application of the A-p from the plug, and the D-p is released, eluted or applied to reverse one or more properties of the conduit to the first configuration.

In certain embodiments, the kit includes the A-p, the B-d, the C-d and the D-p. The B-d and the C-dare administered in a sequential order, wherein the B-d enables transport of the C-d, the C-d enables release of the A-p from the plug, and the D-p is released, eluted or applied to reverse one or more properties of the conduit to the first configuration.

In certain embodiments, the kit includes the A-d, the B-p, the C-p and the D-p. The A-d is administered, wherein activation of the C-p causes release, elution or application of the B-p from the plug, the B-p that is released, eluted or applied promotes the transport of the A-d, and the D-p is released, eluted or applied to reverse one or more properties of the conduit to the first configuration.

In certain embodiments, the kit includes the A-d, the B-p, the C-p and the D-p. The A-d is administered, wherein activation of the C-p causes release, elution or application of the B-p from the plug, the B-p that is released, eluted or applied promotes the transport of the A-d, and the D-p is sequentially released, eluted or applied from the plug to reverse one or more properties of the conduit to the first configuration.

In certain embodiments, one or more of the reversal agent D-d, the reversal agent D-t, and the reversal agent D-p is configured to dissolve the lubricating fluid from a part of the inner surface of the conduit.

In certain embodiments, one or more of the priming agent B-d, the priming agent B-t, the priming agent B-p, the activating agent C-d, the activating agent C-t, and the activating agent C-p causes an exothermic reaction, the exothermic reaction modifies a surface tension between the therapeutic agent and the conduit.

In certain embodiments, one or more of the reversal agent D-d, the reversal agent D-t, and the reversal agent D-p causes an endothermic or exothermic reaction, the endothermic or exothermic reaction causes one of a transitory modification or a permanent modification of a surface tension between the therapeutic agent and the conduit.

In certain embodiments, the priming agent B-t further comprises one or more of a crosslinking shape memory polymer compound, a gelating shape memory polymer compound, and a crosslinking agent.

In certain embodiments, one or more of the reversal agent D-d, the priming agent D-t, and the priming agent D-p is a colloidal suspension. The colloidal suspension is adsorbed on the inner surface of the conduit to enable one of a dewetting or a pinning of any of the therapeutic agent A-d, the therapeutic agent A-t, or the therapeutic agent A-p.

In certain embodiments, one or more of the therapeutic agent A-d, the therapeutic agent A-t, the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p causes a phase transformation of the plug. The phase transformation of the plug enables a transport of one or more of the the therapeutic agent A-d, the therapeutic agent A-t, and the therapeutic agent A-p.

In certain embodiments, the drug formulation further includes a solid agent and a liquid agent. The liquid agent is capable of precipitating out the solid agent from the drug formulation.

In certain embodiments, the drug formulation further includes a solid agent and a liquid agent. The liquid agent is capable of dissolving the solid agent.

In certain embodiments, the drug formulation further includes a solid agent and a liquid agent. The liquid agent modifies a physicochemical property of the solid agent.

In certain embodiments, one or more of the drug formulation and the therapeutic formulation is capable of modifying a frictional drag force between one or more of the therapeutic agent A-d, the therapeutic agent A-t, and the therapeutic agent A-p, and the conduit surface from a first value to a second value.

In certain embodiments, the reversal agent is capable of reversing the frictional drag force between one or more of the therapeutic agent A-d, the therapeutic agent A-t, and the therapeutic agent A-p and the conduit surface from the second value to the first value.

In certain embodiments, one or more of the drug formulation, the therapeutic formulation and the plug formulation includes a shear thinning fluid.

In certain embodiments, the shape of the conduit is selected from the group consisting of a tube, a shunt, a stent, a block, a valve, a tab, a wire, a shell, a wick, a channel, a cable, a hose, a pipe, a catheter, a spiral, a helix, a cone, an oval, a duplex shape having two separate concentric transport channels from the proximal and distal ends, a triplex shape having three separate concentric transport channels from the proximal and distal ends, and a combination thereof.

In certain embodiments, a material of the conduit is selected from the group consisting of non-biodegradable compounds, biodegradable compounds, polyurethane, polyurethane copolymer, fluoropolymer, polyolefin, silicone rubber, hydrophobic polyanhydrides, polyorthoesters, polyphosphazenes, polyphosphoesters/phosphoesters, pseudopolyamino acids, poly(ethylene oxide), PEO, and poly(butylene terephthalate), PBT, biodegradable polymers with hydrolysable chemical bonds, wetting agents, emulsifiers, lubricants, sodium lauryl sulfate, calcium, and magnesium stearate, coloring agents, release agents, coating agents, sweetening, flavoring and perfuming agents, preservatives and antioxidants, poloxamers, polyamides, polyimides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene, polyvinylpryrrolidone, alginate, poly(caprolactone), dextran and chitosan, poly(lactic-co-glycolic acid) or PLGA, polycaprolactone or PLC, Gelatin, DNA hydrogen, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and a combination thereof.

In certain embodiments, a material of the conduit is selected from the group consisting of biodegradable polymers, poly(ethylene vinyl acetate), poly(vinyl acetate), silicone polymers, polyurethanes, polyamides, polyimides, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, copolymers, metals, cobalt, chromium, gold, nickel, platinum, stainless steel, titanium, tantalum, nickel-titanium, alloys, and a combination thereof.

In certain embodiments, a material of the conduit is selected from the group consisting of hydrogels, polyvinyl alcohol, polyvinyl pyrrolidone, polyethylene oxide, polyhydroxyethyl methacrylate, hydroxypropylmethyl cellulose and hydroxyethyl cellulose, polyesteramides, polyglycolic acid, polyvinyl alcohol, copolymers of polyethylene oxide/polylactic acid, copolymers of glycolide/lactide, poly-L-lactic acid compounds, polycarbophil, polyvinyl pyrrolidone, polyvinyl alcohol, hydroxyethyl cellulose, hydroxypropylmethyl cellulose, and a combination thereof.

In certain embodiments, a material of the conduit is selected from the group consisting of gels and polymers with stimuli-responsive functionality, thermoresponsive gels, PNIPAAm and its copolymers with hydrophobic or hydrophilic comonomers, copolymer of n-isopropylacrylamide and at least one acrylic and/or methacrylic monomer such as an alkyl acrylate, methyl acrylate, ethyl acrylate, butyl acrylate or 2-ethylhexyl acrylate, an acrylamide, acrylic acid or salt, 2-ethylacrylic acid, 2-propylacrylic acid, N-acryloxysuccinimide, methacrylic monomers, methacrylate, 2-hydroxymethacrylate, hydroxyethyl methacrylate, butyl methacrylate, methyl ether methacrylate, methyl methacrylate, methacrylamide, methacrylic acid and salt, acrylate monomer modified with poly(ethylene glycol), methacrylate monomer modified with poly(ethylene glycol), and a combination thereof.

In certain embodiments, a material of the conduit is selected from the group consisting of biostable or bioabsorbable polymers, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyimides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, chitins, and a combination thereof.

In certain embodiments, a material of the conduit is selected from the group consisting of non-polymers, sterols, cholesterol, stigmasterol, β-sitosterol, and estradiol; cholesteryl esters such as cholesteryl stearate, C12-C24 fatty acids, lauric acid, myristic acid, palmitic acid, stearic acid, arachidic acid, behenic acid, and lignoceric acid, C18-C36 mono-, di- and triacylglycerides, glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate, glyceryl monostearate, acetylated monoglycerides, sucrose fatty acid esters, sucrose distearate and sucrose palmitate, sorbitan fatty acid esters such as sorbitan monostearate, sorbitan monopalmitate, sorbitan tristearate, C16-C18 fatty alcohols, cetyl alcohol, myristyl alcohol, stearyl alcohol, cetostearyl alcohol, esters of fatty alcohols and fatty acids, cetyl palmitate, cetearyl palmitate, anhydrides of fatty acids, stearic anhydride, phospholipids including phosphatidylcholine (lecithin), phosphatidylserine, phosphatidylethanolamine, phosphatidylinositol, and lysoderivatives thereof, sphingosine and derivatives thereof, sphingomyelins, stearyl, palmitoyl, tricosanyl sphingomyelins, ceramides, stearyl, palmitoyl ceramides, glycosphingolipids, lanolin, lanolin alcohols, and a combination thereof.

In certain embodiments, a material of the conduit is selected from the group consisting of silicones, fluoroplastics, Nylon, polyethylene terephthalate, Polycarbonate, Acrylonitrile Butadiene Styrene, Poly(p-phenylene oxide), Polybutylene terephthalate, Acetal, Polypropylene, Polyurethane, Polyetheretherketone, hydroxylpatite, Ultra-high molecular weight polyethylene, High Density Polyethylene, Low Density Polyethylene, Polystyrene High Impact, Polysulfone, Polyvinylidene fluoride, polystyrene, polymethylmethacrylate, latex, polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, polyelectrolyte hydrogels, hydrogel-forming materials, acrylate, polyacrylate, methacrylic acid, (dimethylamino)ethyl methacrylate, hydroxyethyl methacrylate, poly(vinyl alcohol)/poly(acrylic acid), 2-acrylamido-2-methylpropane sulfonic acid, [(methacrylamido)-propyl]trimethyl ammonium chloride, poly(N-vinyl-2-pyrrolidone/itaconic acid), nonionic hydrogels, poly(ethylene glycol), ethylene glycol diacrylate, polyethylene glycol diacrylate poly(ethylene oxide), diacrylate, acrylamide, polyacrylamide, methylenebisacrylamide, N-isopropylacrylamides, poly(vinyl alcohol), hydrogels made of natural materials, hydrogels made of proteins collagen and silk) and polysaccharides (e.g., chitosan, dextran and alginate), superparamagnetic iron oxide nanoparticles (SPION), cubosomes, polymerosomes, and combinations thereof. In some embodiments, the conduits can be made of metals or metal oxides.

In certain embodiments, a material of the conduit is selected from a group consisting of metals, Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Pb, Bi, La, Ce, Pr, Nd, Sm, Eu, Gd, Tb, Dy, Ho, Er, Tm, Yb, Lu, and their oxides, and a combination thereof.

In certain embodiments, a material of the conduit is selected from a group consisting of metal foams, porous metallic substrates, and a combination thereof.

In certain embodiments, a material of the conduit includes colloidal particles. The colloidal particles are selected from a group consisting of polymethylmethacrylate, melamine resins, silica, polydivinylbenzene microparticles, poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, halogenated polymers, hydrogels, organogels, random and block copolymers, branched, star and dendritic polymers, supramolecular polymers, biopolymer colloid, protein- or polysaccharide-based material, silk fibroin, chitin, shellac, cellulose, chitosan, alginate, gelatin, metals, gold, palladium, platinum, silver, copper, rhodium, ruthenium, rhenium, titanium, osmium, iridium, iron, cobalt, or nickel, oxides, silica, alumina, beryllia, noble metal oxides, platinum group metal oxides, titania, tin oxide, zirconia, hafnia, molybdenum oxide, tungsten oxide, rhenium oxide, vanadium oxide, tantalum oxide, niobium oxide, chromium oxide, scandium oxide, yttria, lanthanum oxide, ceria, thorium oxide, uranium oxide, other rare earth oxides, ferromagnetic particles, ferrimagnetic particles, superparamagnetic particles, iron nanoparticles, nickel nanoparticles, cobalt nanoparticles, charged particles, uncharged particles, hydrophilic particles, hydrophobic particles, amphiphilic particles, and a combination thereof.

In certain embodiments, a precursor material is added to the composition of the conduit. The precursor material is selected from the group consisting of small molecules, dispersed liquid droplets, microparticle and nanoparticle fillers, talc, calcium carbonate, calcium phosphate, anti-oxidants, UV stabilizers, plasticizers, anti-static agents, porogens, slip agents, processing aids, foaming or antifoaming agents, nucleating agents and fillers, and a combination thereof.

In certain embodiments, the shape of the plug is selected from the group consisting of a valve, a membrane, a film, a lid, a handle, an inclusion, a pellet, a cage, a capsule, a powder, a foam, a gel, and a combination thereof.

In certain embodiments, a material of the plug is selected from the group consisting of stimuli-responsive polymer, a gas-selective mobile membrane, stimuli-responsive cilia-like and hair-like fibers, platelets, pillars, reconfigurable tunable nano- or microstructures with functionalized tips or functionalized pillars, and combinations thereof.

In certain embodiments, a material of the plug is selected from the group consisting of poly(lactic-co-glycolic acid) or PLGA, polycaprolactone or PLC, Chitosan, Gelatin, DNA hydrogen, acetalated dextran, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polycarbonate, polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine), and combinations thereof.

In certain embodiments, a material of the plug is selected from the group consisting of commercially available salts containing anions such as acetate, iodide, benzenesulfonate, isethionate, benzoate, lactate, Bbicarbonate, lactobionate, bitartrate, malate, bromide, maleate, calcium edetate, Mandelate, Carbonate, Methylbromide, Chloride, Methylnitrate, Dihydrochloride, Mucate, Edetate, Napsylate, Edisylate, Nitrate, Esylate, Pantothenate, Fumarate, Phosphateldiphosphate, Gluceptate, Polygalacturonate, Gluconate, Salicylate, Glutamate, Stearate, Glycollylarsanilateg, Subacetate, Hexylresorcinate, Succinate Hydrabamine, Sulfate, Hydrobromide, Tannate, Hydroxynaphthoate, Teoclate, Triethiodide, or cationis such as Benzathid, Aluminum, chloroprocaine, Calcium, Choline, Lithium, Diethanolamine, Magnesium, Ethylenediamine, Potassium, Meglumine, Sodium, Procaine, Zinc, and combination thereof.

In certain embodiments, the stimuli-responsive polymer of the plug is selected from a group consisting of nematic, smectic, chiral, discotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

In certain embodiments, the plug comprises an extension. The extension is in form of one or more of a sponge, absorbing pad, foam, receptacle, mesh, porous cladding, inflatable balloons, catheter, fiber, sieve, uniformly or non-uniformly expandable gel, roll, swirl, ball, wrap, film, and dressing.

In certain embodiments, one or more of the conduit and the plug is a microporous absorbent media. The absorbent media is configured to guide and release one or more of the therapeutic agent A-d, the therapeutic agent A-t, and the therapeutic agent A-p.

In certain embodiments, a material of the absorbent media is selected from a group consisting of biocompatible polymers, bioerodable materials, elastomers, metals, metal alloys, glasses, laminates of hydrophilic polymers and hydrophobic polymers, multilaminates or polymer, metals, and/or glasses, biocompatible polymeric materials, homopolymers and copolymers of vinyl acetate, ethylene vinyl acetate copolymer; homopolymers and copolymers of acrylates, poly(methyl) methacrylate (PMMA), polyethylmethacrylate, ethylene glycol dimethacrylate, ethylene dimethacrylate and hydroxymethyl methacrylate, polyurethanes; polyethylenes; polyvinylchlorides; polycarbonates; polyamides; polysulfones; polyesters; polyimides; halogenated polymers, polytetrafluoroethylene (PTFE), polyvinyl fluoride, polychlorotrifluoroethylene, copolymers tetrafiuoroethylene and hexafluoropropylene, PFA, polyolefins, high density polyethylene (HDPE), low density polyethylene (LDPE), linear low density polyethylene (LLDPE), polypropylenes, polystyrenes, nylons; urethanes; homopolymers and copolymers of acrylonitrile, acrylonitrile-butadiene-styrene polymer, styrene acrylonitrile, polycarbonate-acrylonitrile-butadiene-styrene, polyvinylpyrrolidone; 2-pyrrolidone; polyacrylonitrile butadiene, cellulose acetate; polyethylene terephthalate; polymethylpentene; polyisobutylene, polymethylstyrene, polyvinylidine chloride and homopolymers and copolymers of polyvinylidine chloride, polyvinylchloride-acrylic copolymers; PEBAX™; HYTREL™, biocompatible elastomers, silicone rubbers, hydrogels, polyvinyl chloride elastomers; polyolefins, homopolymeric and copolymeric elastomers, urethane-based elastomers; natural rubbers; and fluorinated polymers, PTFE, metallic materials, titanium, platinum, tantalum, gold and their alloys, gold-plated ferrous alloys, platinum-plated titanium, stainless steel, tantalum, gold and their alloys, ferrous alloys, cobalt-chromium alloys, titanium nitride-coated stainless steel, titanium, platinum, tantalum, gold, and their alloys, TEFLON™, nickel titanium, superelastic nickel titanium, and combination thereof.

In certain embodiments, the absorbent media is configured to enhance the flow rate of the transport of one or more of the therapeutic agent A-d, the therapeutic agent A-t, and the therapeutic agent A-p.

In certain embodiments, one or more of the conduit and the plug includes one or more of a chemical sensor, a magnetic sensor, an electrical sensor, and an optical sensor.

In certain embodiments, one or more of the chemical sensor, the magnetic sensor, the electrical sensor, and the optical sensor measures the concentration of one or more of therapeutic agent A-d, the therapeutic agent A-t, and the therapeutic agent A-p, the priming agent B-d, the priming agent B-t, the priming agent B-p, the activating agent C-d, the activating agent C-t, the activating agent C-p, the reversal agent D-d, the reversal agent D-t, and the reversal agent D-p.

In certain embodiments, the conduit is configured to be positioned in an ear, the conduit including: a proximal end configured to be received in an ear canal, the proximal end having a proximal end radius, a distal end opposite the proximal end, the distal end configured to be received in a middle ear, the distal end having a distal end radius, an inner surface connecting the proximal end and the distal end, at least a portion of the inner surface including a conical or curved geometry extending at least partially between the proximal end and the distal end, and an outer surface connecting the proximal end and the distal end; the distal end radius is larger than the proximal end radius.

Although certain embodiments of the present disclosure discuss tympanostomy conduits, it shall be understood that the systems and kits described herein can be used for other medical and biological purposes outside of the middle and inner ear and for treatment of other diseases in different parts of human body, such as catheters, shunts, stents, tubes, and other conduit-like implantable and non-implantable medical devices and combination products. Non-limiting examples include inner ear conduits, prostatic and biliary stents, naso- and oro-intestinal catheters, sinus cavities, stents for sinus cavities, abdominally-based drains, such as drainage of gallbladder, pancreas, intestine; ventricular catheters, intra-abdominal shunt catheters, glaucoma shunts, nasolacrimal stents, central venous catheters; urinary catheters, vascular catheters, and other conduits.

### BRIEF DESCRIPTION OF THE FIGURES

FIG. 1A is a schematic of the kit for optimized drug transport, in accordance with certain embodiments.

FIG. 1B is another schematic of the kit for optimized drug transport, in accordance with certain embodiments.

FIG. 2 illustrates another schematic of the kit for optimized drug transport in accordance with certain embodiments. The drug formulations contain one or more of the following compounds: a therapeutic compound drug, a priming agent, an activating agent, and a reversal agent applied onto the responsive, compound-loaded conduit, that (individually or at the same time) contains a therapeutic compound drug, a priming agent, an activating agent, and a reversal agent, in accordance with certain embodiments. The conduit may further contain a stimuli-responsive “plug” with compounds: a therapeutic compound drug, a priming agent, an activating agent, and a reversal agent.

FIG. 3 illustrates a flowchart of the pathway for a conduit exhibiting self-sustaining release from a therapeutic matrix or a plug with formulation in combination with the application of a topical therapeutic, in accordance with certain embodiments.

FIG. 4A is a phase plot of Ohnesorge number in the surface tension—dynamic viscosity space for a unidirectional conduit design, in accordance with certain embodiments.

FIG. 4B is a phase plot of Weber number in the surface tension—dynamic viscosity space for a unidirectional conduit design, in accordance with certain embodiments.

FIG. 5 illustrates a conduit with a thin permeable or semi-permeable membrane that allows two drug components to be administered on separate parts of the conduit, in accordance with certain embodiments.

FIG. 6 is a schematic showing drug formulations containing one or more of the following compounds: a therapeutic compound, a priming agent, an activating agent, and a reversal agent applied onto the non-responsive, non-therapeutic-loaded conduit, in accordance with certain embodiments.

FIG. 7 is a schematic of drug medium containing an immiscible priming agent that modifies the surface of the conduit to facilitate the drug transport, in accordance with certain embodiments.

FIG. 8A is a schematic showing how the conduit matrix can be embedded with one or multiple materials that, upon release, can facilitate the drug transport, in accordance with certain embodiments.

FIG. 8B illustrates the effect of application of a priming agent B-d onto the conduit: i) the images confirm absence of transport of A-d (ofloxacin) through the tube, whereas ii) the images show how the application of the priming agent B-d allows for an immediate transport of the A-d component through the conduit.

FIG. 9 is a schematic showing how the priming agent or a “stimulus” can be introduced after the drug formulation as a stimuli responsive agent, in accordance with certain embodiments.

FIG. 10 illustrates the administration of a releasing agent of a specific viscosity or surface tension to enable the release of drugs encapsulated within the matrix and a self-regulated transport of drug into the middle ear space to prevent overdosing or underdosing, in accordance with certain embodiments.

FIG. 11 is a schematic showing drug formulations containing one or more of the following compounds: a therapeutic compound drug, a priming agent, an activating agent, and a reversal agent applied onto the responsive, non-therapeutic-loaded conduit containing an activating agent that has the ability to “activate” the conduit on demand, in accordance with certain embodiments.

FIG. 12 illustrates a flowchart of the pathway of one or more therapeutic agents and additives in a topically applied therapeutic drug, in accordance with certain embodiments.

FIG. 13 illustrates fluid transport through the conduit facilitated by the Marangoni effect, in accordance with certain embodiments. Added drop of low surface tension spreads towards high surface tension surface on conduit and facilitates transport of therapeutic fluid.

FIG. 14 shows a series of photographs demonstrating the fluid transport of antibiotic drops Ciprodex® through the optimized conduit with 0.60 mm diameter (i) and lack of any transport of antibiotic drops through the silicone conduit with conventional cylindrical geometry, in accordance with certain embodiments.

FIG. 15 illustrates no fluid transport of water through the optimized curved geometry of the silicone-oil infused silicone elastomer conduit with 0.60 mm diameter, in accordance with certain embodiments.

FIG. 16A demonstrates a process of a formation of a solid biodegradable plug D-p made of solidified egg whites in air within the conduit lumen on demand. The solid plug was used to prevent water from entering the conduit.

FIG. 16B illustrates the plug D-p being removed on demand by applying ofloxacin to restore the fluid transport capabilities.

FIG. 17 demonstrates a process of a formation of liquid plug D-p by applying a high-viscosity silicone fluid and the consequent removal of D-p, by applying the priming agent B-d (a low-viscosity silicone fluid), to restore the transport of A-d through the tube.

FIG. 18 is a schematic of experiments indicating feasibility of assessing drug elution in vivo in chinchillas, in accordance with certain embodiments.

FIG. 19A illustrates middle ear and external auditory canal (EAC) endoscopic visualization of drug elution through the optimized conduit—the liquid-infused tympanostomy tube with ID=0.66 mm, “LI-TT” (i) and control conduit—Summit Medical VT-1002-01, sterile commercial silicone Collar Button, ID=1.27 mm, “SM-TTs” (ii), in which Ciprodex® can be seen as it rapidly progresses into the middle ear with the LI-TT leaving no drug pool in the EAC (i), as opposed to no Ciprodex® travelling through the SM-TT, and rather staying within the EAC (ii), in accordance with certain embodiments.

FIG. 19B compares mass of ciprofloxacin in middle ear through sequential bullar rinses after drug elution through the optimized LI-TT, (denoted as the optimized drug-tube combination in the plot) and control SM TT (denoted as the conventional design in the plot), as assessed by liquid chromatography-mass spectrometry (LC-MS), in accordance with certain embodiments. N/A indicates an amount of drug below the detection limit of 100 ng/ml. Chinchillas underwent a sequential series of middle ear saline washes, and middle ear fluid was aspirated for the subsequent analysis by LC-MS.

FIG. 20 shows the Kaplan-Meier curve for the probability of elution of the first drop of Ciprodex® through the LI-TT and SM-TT conduits (LI-TT, denoted as Test TT, and SM-TT, denoted as SoC TT) versus time. The plot takes into account user-defined censoring of data at 5 min after the start of observation, and, in the inset, the corresponding mass of ciprofloxacin in fluid aspirated from the middle as detected by LCMS, in accordance with certain embodiments.

### DETAILED DESCRIPTION OF THE INVENTION

The disclosed subject matter relates to a drug delivery device and a drug delivery method for the treatment of middle ear (e.g., acute otitis media, otitis media with effusion) and/or inner ear disorders (including but not limited to sudden hearing loss, noise-induced hearing loss, progressive hearing loss, aminoglycoside-induced hearing loss, presbycusis, tinnitus, Ménière's disease, autoimmune inner ear disorder, and infections, dizziness, vertigo, disequilibrium, and pre-syncope, Ramsay Hunt's Syndrome, mal de debarquement, benign paroxysmal positional vertigo, labyrinthitis, vestibular neuritis, viral neuronitis, labyrinthitis, viral endolymphatic labyrinthitis, drug-induced ototoxicity, endolymphatic hydrops, head trauma with lesional vestibular deficits, labyrinthine haemorrhage, chronic or acute labyrinthine infection, serous labyrinthine, barotraumatism, autoimmune inner ear disease, presbyvestibulia, and toxic vestibular impairments). In order to treat these disorders, it is often desirable to administer therapeutic agents, e.g., medications or other medical fluids, into the middle and inner ear of a patient, and the delivery of medicines to such structures is often of primary importance. Typical clinical practice involves either oral, venous, or arterial drug delivery, or topical drug delivery. Topical access to the inner ear may be achieved through a variety of middle-inner ear interface tissue structures, such as the tympanic membrane, the round window membrane, the oval window/stapes footplate, the annual ligament or the endolymphatic sac/endolymphatic duct.

Although certain embodiments of the present disclosure discuss tympanostomy conduits, it shall be understood that the systems and kits described herein can be used for other medical and biological purposes outside of the middle and inner ear and for treatment of other diseases in different parts of human body, such as catheters, shunts, stents, tubes, and other conduit-like implantable and non-implantable medical devices and combination products. Non-limiting examples include inner ear conduits, prostatic and biliary stents, naso- and oro-intestinal catheters, sinus cavities, stents for sinus cavities, abdominally-based drains, such as drainage of gallbladder, pancreas, intestine; ventricular catheters, intra-abdominal shunt catheters, glaucoma shunts, nasolacrimal stents, central venous catheters; urinary catheters, vascular catheters, and other conduits.

It has become routine for otolaryngologists to perform intratympanic injections, and the efficacy of this approach compared to the systemic drug administration has been confirmed for various clinical indications. However, there remains a considerable variability in clinical outcomes. This approach can be limited by the size and characteristics of therapeutic substance's molecules (molecular weight, concentration, liposolubility, electrical charge), and is dependent on the frequency of drug administration into the middle ear as well as on the physical properties of the round window membrane (e.g., thickness). Several (even more invasive) approaches have been focused on overcoming the variability of the diffusion-limited drug delivery via intratympanic injections. For example, one of the approaches invokes a plurality of micro-needles which can be delivered to the round window membrane by a delivery device. Intratympanic delivery of drugs involves making an incision in the ear canal, i.e., anesthetized tympanic membrane (ear drum) to create an access point to the middle ear. Once the access is available, the medical provider can inject the drug into the middle ear in liquid form and allow it to be absorbed into the inner ear by diffusion across the round window membrane. Another method is based on a system for inner ear drug delivery via trans-round window membrane injection and apparatus for the controlled and site-specific transfer, e.g., “microdosing”, of fluid materials into and out of the inner ear, or via round window catheters, microimplants, or osmotic pumps.

Less invasive and more convenient methods for the patient include placing a ventilation (tympanostomy) tubular conduit in the tympanic membrane and then having the patient self-dispense the drug into the outer ear canal whereby it is intended to pass through the conduit (tube) into the middle ear, and thence the inner ear. For example, for patients at high risk for middle ear infections, antibiotics may also be administered through the tympanostomy conduit in a prophylactic manner. Traditionally, this method has been considered to be unreliable for passage of therapeutics because the passage of the liquid into the middle ear can be inhibited by the surface tension of the liquid. The miniature, mm-scale radius of the conduit lumen presents a significant barrier to drug permeation, which is further exacerbated by bio adhesion-associated clogging. Strategies to coerce therapeutic droplets through tympanostomy conduits using tragal pumping, in which the tragus is pressed repeatedly to induce transient pressure gradients, rely exclusively on patient training and compliance (which poses a major challenge for young patients). Thus, a complete and reliable delivery of therapeutic drugs remains a challenge in existing tympanostomy conduits. In the case of topical antibiotics, suboptimal dosing of antibiotics can not only prolong infection but also promote the selection of antibiotic-resistant bacteria. Although there have been many research advances in materials science and surface chemistry, there has not been a meaningful upgrade to tympanostomy conduits. Previous attempts to improve the drug delivery efficaciousness of the tympanostomy conduits were primarily focused on drug elution mechanisms from the conduit matrix, e.g., in bio-absorbable ventilation conduits that are designed with a specific degradation rate. Alternatively, the conduits were proposed to be used with the Silverstein Microwick, allowing for repeated dosing of commercially available medications, or packaging or suspending the drug in a vehicle that permits sustained exposure to the middle ear. The pharmacokinetic profiles achievable with this device are largely to be determined and are ultimately limited by the behavior/characteristics of the wick. Using the wick may result in additional surgical efforts (and costs) to place and subsequently remove it, with an uncontrollable risk of overdosing the inner ear. Furthermore, such devices do not easily allow for selective transport of fluid into the middle ear space; for example, they may not prevent exogenous water from entering the ear, posing an infection risk.

As mentioned above, the currently available drug delivery methods for middle and inner ear diseases carry the risk of attendant side effects. They can be invasive and risky, require multiple daily doses (e.g., intratympanic injections or infusions) of drugs, can cause patient discomfort and non-compliance, may introduce infections or other immune disorders as a result of microbial or endotoxin presence, resulting in a permanent structural damage (e.g., perforation of the tympanic membrane) or hearing loss and the like. Therefore, there is a need for a drug delivery method of providing therapeutically effective concentrations of therapeutic agents in the middle and inner ear over a prolonged or other desired period of time, with fluid selectivity and/or at prescribed times.

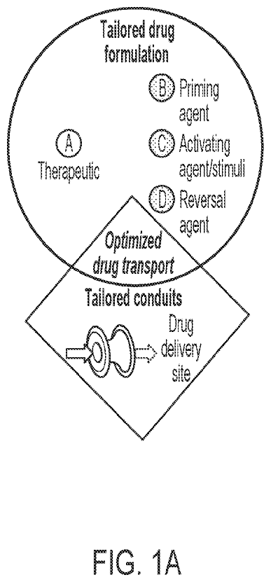

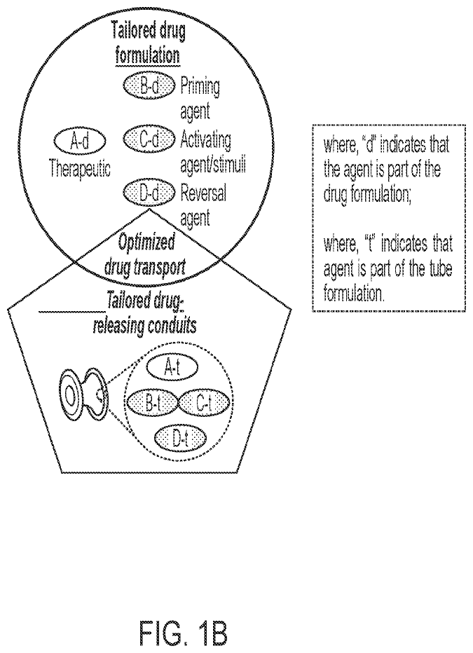

Herein, improved drug delivery devices and methods, such as drug transport for middle ear drugs are provided. In certain embodiments, as shown in FIG. 1A, a drug formulation (DF) can be optimized for drug transport in conduit, depending on the desired characteristics of the drug delivery.

In certain situations, a patient may require the administration of multiple otic drug prescriptions that differ in fluid properties in which changing of the conduit geometry for each drug is infeasible for the patient. Hence, a scheme is presented herein in which the benefits of a customized conduit for delivering a set of therapeutic drugs, pro-drugs or co-drugs, are maintained through delivery of additional agents that modify the conduit (tube) before, during, or after transport of the drugs, pro-drugs or co-drugs. In the same way a single conduit can be optimized for promoting effective transport of a selected set of drugs, so does a kit containing multiple topical formulations can be designed to enable selective transport of a set of drugs within a single conduit. This can be achieved by manipulating the formulation containing the drug, pro-drug and co-drug, for which all existing components including the active components and the carrier medium can be tuned. Alternatively, new agents that serve to prime, activate, or restore the tube for drug transport can be applied before the formulations containing the drug, pro-drug and co-drug, or incorporated directly into the formulation containing the drug, pro-drug and co-drug itself. Furthermore, the conduit can also exhibit controlled or sustained release properties, and/or exhibit useful interactions with topically administered formulations to provide therapeutically effective concentrations of therapeutic agents in the middle ear and inner ear for a desired period of time.

For example, the drug formulations can contain one or more of the following agents:
a therapeutic agent, such as a therapeutic compound drug(s), that needs to be delivered for treatment of a variety of disorders, e.g., ear disorders (denoted as “A-d” where “d” indicates the therapeutic agent is part of the drug formulation DF);
a priming agent that modifies the physical properties of the conduit including size, shape, physical heterogeneity, wettability, surface energy, drag, surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, in preparation for drug transport (denoted as “B-d” where “d” indicates the priming agent is part of the drug formulation);
an activating agent that has the ability to “activate” drug delivery through the conduit tube/drug on demand (either just prior to or during drug administration) through adjusting drug/tube surface interactions (for example, photothermal agents such as gold nanoparticles embedded in the tube or incorporated in the topically administered drug formulation DF that can produce heat locally to reduce the surface tension and promote flow) (denoted as “C-d” where “d” indicates the activating agent is part of the drug formulation); and/or
a reversal agent that reverses, restores, or changes the physical properties of the conduit including size, shape, physical heterogeneity, wettability, surface energy, drag, surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, subsequent to drug transport to prepare the conduit for the next phase of delivery or to prevent exogeneous water transport through the conduit (denoted as “D-d” where “d” indicates the reversal agent is part of the drug formulation).

In certain embodiments, the reversal agent, D-d, by reverting or changing the physical properties of the conduit to an original state, prevents the water transport through the conduit.

In certain embodiments, the components of DF may enter sequentially through a kit containing a set of topical formulations. Following are the examples of combination kits comprising certain components of DF that are administered in a specific sequential order:

1. Agents A-d, and D-d: The components are administered in following sequence (1) a formulation containing A-d that is deposited onto the conduit, and (2) a formulation containing a reversal agent D-d that is applied to modify or close the conduit after transport of A-d to prevent penetration by exogeneous, environmental water and ototoxic compounds.

2. Agents A-d, C-d, D-d: The components are administered in following sequence (1) a formulation containing A-d (2) a formulation containing C-d and (3) a formulation containing D-d. In this sequence, (1) is deposited into the tube, followed by (2) that stimulates the progression of A-d through the tube, followed by (3) that is applied to modify or close the conduit after the transport of the A-d to prevent penetration by environmental water and ototoxic compounds.

3. Agents C-d, A-d, D-d: The components are administered in following sequence (1) a formulation containing C-d that is topically applied to the tube, (2) formulation containing A-d, and (3) formulation containing D-d. In this sequence, C-d deposition through (1) allows the penetration of (2), and can be followed by (3) that is applied to modify or close the tube after transport of the A-d to prevent penetration by environmental water and ototoxic compounds.

4. Agents B-d, A-d, D-d: The components are administered in following sequence (1) a formulation with B-d, such as an oil, (2) a formulation with A-d, and (3) a formulation with D-d. In this sequence, application of (1) promotes the transport of (2), and (3) is applied to modify or close the tube after therapeutic transport to prevent penetration by environmental water and ototoxic compounds.

5. Agents B-d, C-d, A-d, D-d: The components are administered in following sequence (1) a formulation with B-d, (2) a formulation with C-d, (3) a formulation with A-d, and (4) a formulation with D-d. In this sequence, (1) promotes the transport of (2), which can be triggered to allow the penetration of (3), and which can be followed by (4) that is applied to modify or close the tube after therapeutic transport to prevent penetration by environmental water and ototoxic compounds.

6. Agents A-d: A formulation containing A-d that is deposited onto the conduit.

7. Agents A-d, C-d: The components are administered in following sequence (1) a formulation containing A-d, and (2) a formulation containing C-d. In this sequence, (1) is deposited into the tube, followed by (2) that stimulates the progression of A-d through the tube.

8 Agents C-d, A-d: The components are administered in following sequence (1) a formulation containing C-d that is topically applied to the tube, and (2) formulation containing therapeutic compound A-d. In this sequence, C-d deposition allows the penetration of A-d to be triggered.

9. Agents B-d, A-d: The components are administered in following sequence (1) a formulation with B-d, such as an oil, and (2) a formulation with A-d. In this sequence, application of (1) promotes the transport of (2).

10. Agents B-d, C-d, A-d: The components are administered in following sequence (1) a formulation with B-d, (2) a formulation with C-d, and (3) a formulation with A-d. In this sequence, (1) promotes the transport of (2), which can be triggered to allow the penetration of (3).

The drug formulations can be formulated with several compounds that interact synergistically with the conduit to promote drug transport. The interaction of the drug formulation with the conduit (or with the various agents within the drug formulation) can be initiated with certain stimuli, such as physical, chemical, thermal, optical, electrical, or other types of stimuli.

In another embodiment, as shown in FIG. 1B, in addition to or separately from the drug formulation discussed above in FIG. 1A, the conduit can be designed with therapeutic formulations preloaded into the conduit (i.e., tube formulations (TF)) that can interact with the topically administered set of drug formulations, depending on the desired characteristics of drug delivery. The tailored drug formulation (DF) or tube formulations (TF) can be introduced externally or embedded directly into the conduit, respectively, and reapplied multiple times onto the conduit.

In certain embodiments, the conduits can be preloaded with formulation that contains one or more of the following agents:
A therapeutic agent, such as therapeutic compound drug(s), that needs to be delivered for treatment of a variety of disorders, e.g., ear disorders (denoted as “A-t” where “t” indicates the therapeutic agent is part of the tube's (or conduit's) therapeutic formulation);
A priming agent that modifies the physical properties of the conduit including size, shape, physical heterogeneity, wettability, surface energy, drag, surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, in preparation for drug transport (denoted as “B-t” where “t” indicates the priming agent is part of the tube's (conduit's) therapeutic formulation);
An activating agent that has the ability to “activate” drug delivery through the conduit tube/drug on demand (either just prior to or during drug administration) through adjusting drug/tube surface interactions (for example, photothermal agents such as gold nanoparticles embedded in the tube or incorporated in the topically administered drug formulation that can produce heat locally to reduce the surface tension and promote flow) (denoted as “C-t” where “t” indicates the activating agent is part of the conduit's therapeutic formulation); and/or
A reversal agent that reverses or changes the physical properties of the conduit including size, shape, physical heterogeneity, wettability, surface energy, drag, surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, subsequent to drug transport to prepare the conduit for the next phase of delivery or to prevent exogenous water transport through the conduit (denoted as “D-t” where “t” indicates the reversal agent is part of the tube's (or conduit's) therapeutic formulation).

In certain embodiments, the reversal agent, D-t, by reverting or changing the physical properties of the conduit to an original state, prevents the water transport through the conduit.

In certain embodiments, the components of DF and TF interact sequentially. Following are non-limiting examples of combination kits comprising certain components of DF and TF that interact in a specific sequential order:

1. Agents A-d, D-t: The kit comprises (1) a formulation containing A-d that is deposited onto the tube, and (2) a conduit with D-t that is already embedded in the conduit. The components are administered and subsequently interact in following sequence: delivery of (1) causes (2) to elute from the tube, thus modifying or closing the tube after transport of A-d to prevent penetration by environmental water and ototoxic compounds.

2. Agents A-d, C-d, D-t: The kit comprises (1) a formulation containing A-d, (2) a DF formulation containing C-d, and (3) a conduit (tube) containing D-t pre-embedded in the tube. The components are administered and subsequently interact in following sequence: (1) is deposited into the conduit, followed by (2) which stimulates the progression of A-d through the conduit, causing (3) that is pre-embedded in the conduit to elute and thus closing the conduit after transport of A-d to prevent penetration by environmental water and ototoxic compounds.

3. Agents C-d, A-d, D-t: The kit comprises (1) a formulation containing C-d that is topically applied to the tube, (2) a formulation containing A-d and (3) a conduit containing D-t pre-embedded in the tube. The components are administered and subsequently interact in following sequence: deposition of (1) allows the penetration of A-d in (2) to be triggered, causing (3) to be secreted from the conduit and thus modifying or closing the conduit after transport of A-d to prevent penetration by environmental water and ototoxic compounds.

4. Agents B-d, A-d, D-t: The kit comprises (1) a formulation with B-d, such as an oil (2) a formulation with A-d, and (3) a tube with formulation containing D-t reversal agent pre-embedded in the tube. The components are administered and interact in following sequence: application of (1) promotes the transport of (2), and (3) is released to modify or close the tube after transport of A-d to prevent penetration by environmental water and ototoxic compounds.

5. Agents B-d, C-d, A-d, D-t: The kit comprises (1) a formulation with B-d, (2) a formulation with C-d, (3) a formulation with A-d, and (4) a conduit with agent D-t pre-embedded in the tube. The components are administered and interact in following sequence: delivery of (1) promotes the transport of (2), which can be triggered to allow the penetration of (3), and which can be followed by (4) that is released from the conduit to modify or close the tube after transport of A-d to prevent penetration by environmental water and ototoxic compounds.

6. Agents B-d, A-t, D-d: The kit comprises (1) a formulation with B-d, (2) a conduit with A-t embedded, and (3) a DF formulation with a reversal agent D-d. The components are administered and subsequently interact in following sequence: delivery of (1) allows for a therapeutic compound A-t to desorb, be cleaved from, or elute from (2), and can be followed by a reversal agent D-d that is applied to modify or close the conduit after transport of therapeutic agent A-t to prevent penetration by environmental water and ototoxic compounds.

7. Agents C-d, A-t, D-d: The kit comprises (1) a DF formulation with an activating agent C-d, (2) a tube with TF formulation containing therapeutic compound A-t embedded in the conduit, and (3) a DF formulation with a reversal agent D-d. The components are administered and subsequently interact in following sequence: delivery of (1) triggers A-t to be cleaved, decomposed, or elute from the conduit, and can be followed by (3) that is applied to modify or close the tube after transport of A-t to prevent penetration by environmental water and ototoxic compounds.

8. Agents B-d, C-d, A-t, D-d: The kit comprises (1) a formulation with C-d and B-d, (2) a conduit with A-t embedded into it, and (3) a formulation with D-d. The components are administered and subsequently interact in following sequence: delivery and activation of C-d in (1) triggers the transport of promoting agent B-d, triggering the release of therapeutic compound A-t from (2), and can be followed by (3) that is applied to modify or close the conduit after transport of A-t to prevent penetration by environmental water and ototoxic compounds.

9. Agents C-d, B-d, A-t: The kit comprises (1) a formulation with C-d, (2) a formulation with B-d, and (3) a conduit with A-t embedded into it. The components are administered and subsequently interact in following sequence: delivery and activation of C-d in (1) triggers the transport of promoting agent B-d, triggering the release of therapeutic compound A-t from (3).

10. Agents C-d, B-d, A-t, D-d: The kit comprises (1) a formulation with C-d, (2) a formulation with B-d, (3) a conduit with A-t embedded into it, and (4) a formulation with D-d. The components are administered and subsequently interact in following sequence: delivery and activation of C-d in (1) triggers the transport of promoting agent B-d, triggering the release of therapeutic compound A-t from (3), and can be followed by D-d in (4) that is applied to modify or close the conduit after transport of A-t to prevent penetration by environmental water and ototoxic compounds.

11. Agents B-t, C-t, A-d: The kit comprises (1) a conduit with a priming agent B-t and activating agent C-t and (2) a formulation containing A-d. The components are administered and subsequently interact in following sequence: delivery and activation of C-t in (1) causes B-t to elute, which promotes the transport of therapeutic compound A-d in (2) through the conduit.

12. Agents B-t, C-t, A-d, C-d: The kit comprises (1) a tube with a priming agent B-t and activating agent C-t and (2) a formulation containing A-d and C-d. The components are administered and subsequently interact in following sequence: delivery and activation of C-t in (1) causes B-t to desorb or elute from the conduit, which allows A-d to be transported efficiently through the tube in conjunction with a stimulus provided by C-d.

13. Agents B-t, C-t, A-d, B-d, C-d: The kit comprises (1) a tube with a priming agent B-t and activating agent C-t, (2) a formulation containing A-d, B-d and C-d. In this sequence, the conduit already contains necessary agents to facilitate transport. However, they are released upon particular stimulus. In this sequence, the formulation is administered which enters the lumen with one priming agent B-d. This is followed by an external stimulus that enables the conduit to release agents B-t and activate agent C-t that further facilitate the transport of the DF A-d. This sequence can be optionally followed by D-d which reverts the conduit back to its original shape to prevent the transport of environmental water or ototoxic materials. This allows drugs to be administered and released on separate mechanisms.

14. Agents B-d, C-d, A-t, D-t: The kit comprises (1) a formulation containing B-d, (2) a formulation containing C-d, (3) a conduit with A-t and D-t. The components are administered and subsequently interact in following sequence: delivery of (1) allows for the delivery of (2), which when triggered enables (3) containing A-t to be released from the conduit, though the effect of C-d is eventually suppressed due to the continuous presence of D-t which serves to revert the conduit back to its original non-transporting state.

Therapeutic formulations (i.e., tube formulation) can be formulated with several compounds that interact synergistically with the conduit, one or more agents of the drug formulation and/or with one or more other agents of the therapeutic formulation. The interaction of the therapeutic formulation with the conduit, the drug formulation DF and/or with the one or more other agents of the tube formulation TF can be initiated with certain stimuli, such as physical, chemical, thermal, optical, electrical, or other types of stimuli.

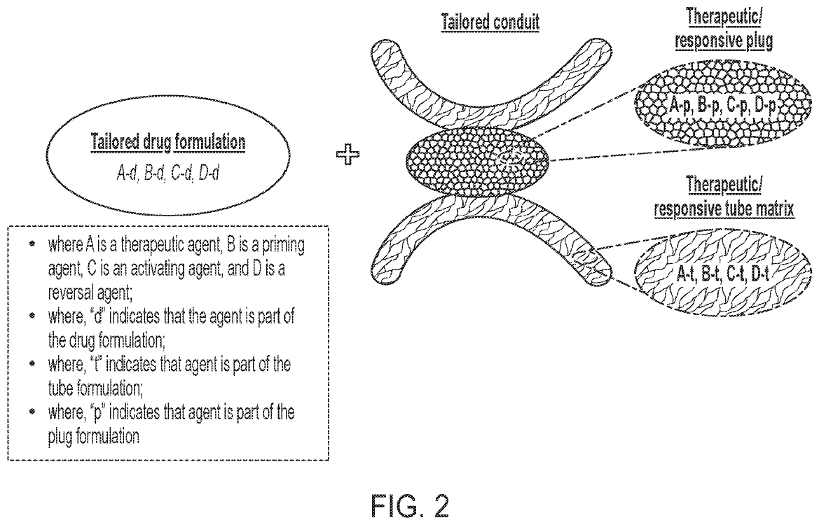

In yet another embodiment, as shown in FIG. 2, in addition to or separately from the drug formulation and the tube formulation discussed above in FIGS. 1A and 1B, the conduit can be designed such that a “plug” can be inserted into the conduit, where the plug is provided with certain plug formulations (PF) that can interact with the drug formulation and/or the therapeutic formulation depending on the desired characteristic of drug delivery.

The plug may be in a number of different forms, such as a valve, a lid, a handle, an inclusion, and the like. The interaction of the plug formulation with the conduit, drug formulation and/or the therapeutic formulation can be initiated with certain stimuli, such as physical, chemical, thermal, optical, electrical, or other types of stimuli.

In certain embodiments, the plug can be preloaded with a plug formulation that contains one or more of the following agents:
A therapeutic agent, such as therapeutic compound drug(s), that needs to be delivered for treatment of a variety of disorders, e.g., ear disorders (denoted as “A-p” where “p” indicates the therapeutic agent is part of the plug formulation);
a priming agent that modifies the physical properties of the conduit including size, shape, physical heterogeneity, wettability, surface energy, drag surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, in preparation for drug transport (denoted as “B-p” where “p” indicates the priming agent is part of the plug formulation);
an activating agent that has the ability to “activate” drug delivery through the conduit tube/drug on demand (either just prior to or during drug administration) through adjusting drug/tube surface interactions (for example, photothermal agents such as gold nanoparticles embedded in the tube or incorporated in the topically administered drug formulation that can produce heat locally to reduce the surface tension and promote flow) (denoted as “C-p” where “p” indicates the activating agent is part of the plug formulation); and/or
a reversal agent that reverses or changes the physical properties of the conduit including size, shape, physical heterogeneity, wettability, surface energy, drag, surface lubricity, porosity, texture, roughness, permeability, swellability, anisotropy, acoustic properties, or combinations thereof; the chemical properties of the conduit including type and degree of chemical functionalization, pH, reactivity, degradability, degree of polymerization, degree of crosslinking, chemical gradient and anisotropy, surface composition, chemical heterogeneity, concentration and ratios of components of different molecular weights, polydispersity, ability to uptake liquids, compatibility with transported liquids, including pharmaceutical formulations, or combinations thereof; the mechanical properties of the conduit including Young's moduli, elasticity, stiffness, hardness, toughness, friction, rigidity, compressibility, sound absorption, or combinations thereof; the optical properties of the conduit including light absorption, light scattering, transmission, or combinations thereof; the thermal properties of the conduit including glass transition temperature, thermal conductivity, thermal expansion, or combinations thereof; the acoustical properties of the conduit including acoustical absorption, speed of sound, sound reflection, sound transfer, elasticity, or combinations thereof, for purposes of fluid transport driven by Surface Acoustic Waves (SAW); the electrical properties of the conduit including capacitance, dielectric constant, resistivity, conductivity, susceptibility, permittivity, piezoelectric constant, or combinations thereof, for electrically driven transport; the magnetic properties of the conduit including Curie temperature, diamagnetism, hall coefficient, hysteresis, magnetoresistance, permeability, piezomagnetism, spin hall effect, or combinations thereof, for magnetically driven flow, subsequent to drug transport to prepare the conduit for the next phase of delivery or to prevent exogenous water transport through the conduit (denoted as “D-p” where “p” indicates the reversal agent is part of the plug formulation).

In certain embodiments, the reversal agent, D-p, by reverting or changing the physical properties of the conduit to an original state, prevents the water transport through the conduit.

In certain embodiments, the components of DF may be deposited sequentially in separate administrations. Following are non-limiting examples of combination kits comprising certain components of DF that are delivered in separate formulations, and interact with components of TF and PF in a specific sequential order:

1. Agents C-d, A-p, D-d: The kit comprises (1) a formulation with activating agent C-d, (2) a conduit with a plug with A-p, and (3) a formulation with a reversal agent D-d. The components are administered and subsequently interact in following sequence: (1) activates the release of therapeutic agent from (2), which is followed by (3) that modifies the conduit to the original state in which A-p is no longer eluting from the plug.

2 Agents B-d, C-d, A-p, D-d: The kit comprises (1) a formulation containing B-d, (2) a formulation containing C-d, (3) a conduit with a plug containing A-p, and (4) a formulation containing D-d. The components are administered and subsequently interact in following sequence: the application of the B-d in (1) allows for transport of (2), which enables therapeutic compound A-p from the (3), and can be followed by application of (4), which reverts the conduit back to its original shape to prevent the transport of (3).

3. Agents C-p, B-p, A-d, D-t: The kit comprises (1) a conduit containing a plug with B-p and C-p, (2) a formulation containing A-d, and (3) a conduit with matrix containing D-t. The components are administered and subsequently interact in following sequence: activation of C-p in the plug allows for B-p to elute in (1), which promotes delivery of A-d in (2), while continuous presence of D-t in the conduit in (3) reverts the action of the plug over time.

4. Agents C-p, B-p, A-d, B-d, D-t: The kit comprises (1) a conduit containing a plug with B-p and C-p, (2) a formulation containing A-d and B-d, and (3) a conduit with matrix containing D-t. The components are administered and subsequently interact in following sequence: activation of C-p in the plug allows for B-p to elute in (1), which promotes delivery of A-d in (2), while continuous presence of D-t in (3) reverts the action of the plug over time.

5. Agents C-d, A-p, D-p: The kit comprises (1) a formulation with activating agent C-d, (2) a conduit with a plug with A-p, and (3) a conduit containing a plug with D-p. The components are administered and subsequently interact in following sequence: (1) activates the release of therapeutic agent from (2), which is followed by (3) that modifies the conduit to the original state in which A-p is no longer eluting from the plug.

6. Agents B-d, C-d, A-p, D-p: The kit comprises (1) a formulation containing B-d, (2) a formulation containing C-d, (3) a conduit with a plug containing A-p, and (4) a conduit containing a plug with D-p. The components are administered and subsequently interact in following sequence: the application of promoting agent B-d in (1) allows for transport of (2), which enables therapeutic compound A-p from the (3), and can be followed by application of (4), which reverts the conduit back to its original shape to prevent the transport of (3).

7. Agents C-p, B-p, A-d, D-p: The kit comprises (1) a conduit containing a plug with B-p and C-p, (2) a formulation containing A-d, and (3) a conduit containing a plug with D-p. The components are administered and subsequently interact in following sequence: activation of C-p in the plug allows for B-p to elute in (1), which promotes delivery of A-d in (2), while D-p in (3) reverts the action of the plug over time.

8. Agents C-p, B-p, A-d, B-d, D-p: The kit comprises (1) a conduit containing a plug with B-p and C-p, (2) a formulation containing A-d and B-d, and (3) a conduit containing a plug with D-p. The components are administered and subsequently interact in following sequence: activation of C-p in the plug allows for B-p to elute in (1), which promotes delivery of A-d in (2), while D-p in (3) reverts the action of the plug over time.

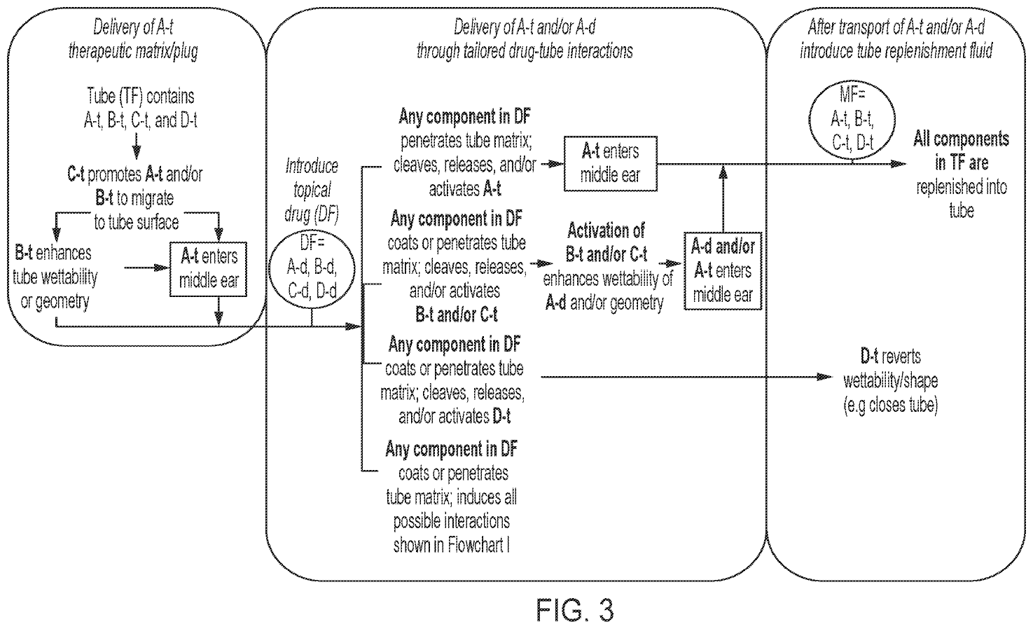

FIG. 3 shows a representative flowchart of various possible interactions between one or more agents of the tube formulation (TF) with one or more agents of the drug formulation (DF) to achieve a sustained drug release and enhanced drug formulation flux through the conduit. Although the interaction between the plug formulation PF with the drug formulation DF, therapeutic formulation TF, and/or conduit is not explicitly discussed, one skilled in the art would readily be able to recognize similar interactions can be achieved.

In certain embodiments of FIG. 3, TF may include a combination of A-t, B-t, C-t, and/or D-t, where “Component-t” is a component initially placed within the matrix or lumen of the conduit. In the presence of a stimulus, C-t can be activated to release (i.e. cause migration) of A-t and/or B-t to the inner surface of the conduit. In situations where B-t migrates to the inner surface of the conduit, B-t is configured to enhance the wettability or size, or shape or porosity of the conduit that enables transport of A-t to the middle ear. In some embodiments, the wettability or size, or shape or porosity of the conduit is configured such that it enables the transport of A-t that has migrated to the surface of the conduit. The components within TF may also self-release, without the stimulus provided by C-t.

In certain embodiments of FIG. 3, the conduit may exhibit self-sustaining release from the therapeutic matrix or the plug with formulation TF (including a combination of A-t, B-t, C-t, and/or D-t) in combination with the application of a topical therapeutic DF (including a combination of A-d, B-d, C-d, and/or D-d), where “Component-d” is a component that is externally deposited into the lumen or matrix of the conduit through topical drug.

For example, one or more of the components of DF may be introduced concurrently in a single suspension to undergo a single or a sequence of interactions with one or more of the components of TF. For example, one or more components of DF may penetrate the conduit matrix, causing release and/or activation of A-t via one or more of disassembly, linker cleavage, swelling, cap removal or gas generation mechanisms. This results in a transport and eventual delivery of A-t to the middle ear. In another example, one or more components of DF may coat or penetrate the conduit matrix, causing cleavage, release, and/or activation of B-t and/or C-t. The components B-t and/or C-t can further modify the wettability or size, or shape or porosity of the conduit to enable transportation and delivery of A-d and/or A-t to the middle ear. In another embodiment, one or more components of DF may coat or penetrate the conduit matrix, causing cleavage, release, and/or activation of D-t. The activated D-t reverses or changes conduit shape, size, porosity or wettability after drug transport to prepare it for the next phase of delivery.

In certain embodiments, all the components of DF may enter sequentially.

After the delivery of active components is completed, the components that have exhausted can be replenished via a matrix replenishment fluid (MRF), which can contain any of the components from TF and/or DF. Components embedded within the matrix of the conduit can be replenished through application of a matrix replenishment fluid, which contains moieties that diffuse or can be convected through the matrix of the conduit material. In some cases, these moieties can cause swelling of the conduit material, facilitating the replenishment process.

The present disclosure can provide a higher degree of control over rate and sequence of delivery for the therapeutic components of interest, depending on: 1) whether a single or multiple drugs are administered to the patient's ear for one particular indication, and 2) on the drug administration modality (i) topical administration via ear drops, gels, or other formulations ear drops vs ii) sustained release from the conduit directly, where (i) and (ii) are not mutually exclusive. The implementation can further be adjusted by various design restraints, including but not limited to e.g., minimization of the conduit radius to reduce invasiveness, utilization of materials that prevent the irritability of the tissue, and others. The above-described scenarios for an abstract patient are discussed as case studies in below sections.

In general, the properties of a fluid material that is transported across the conduit may vary within the space of surface tension and dynamic viscosity. Reynolds number is defined as the ratio of inertial forces to viscous drag forces within a fluid, while Weber number is the ratio of kinetic energy of a fluid to its surface energy. These dimensionless numbers can be compared in a ratio to understand the overall behavior of the fluid being transporting through a conduit using Ohnesorge number (Oh), which relates the viscous forces to inertial and surface tension forces. Although the Ohnesorge number is typically used to determine jettability of fluids from inkjet printers, the concepts of fluid properties can be extended to understanding transport and depinning of fluids, such as drugs, from sub-annular conduits. It can be defined as:

$$Oh = \frac{\sqrt{We}}{Re} = \frac{μ}{\sqrt{ρ σ l}} ,$$

where

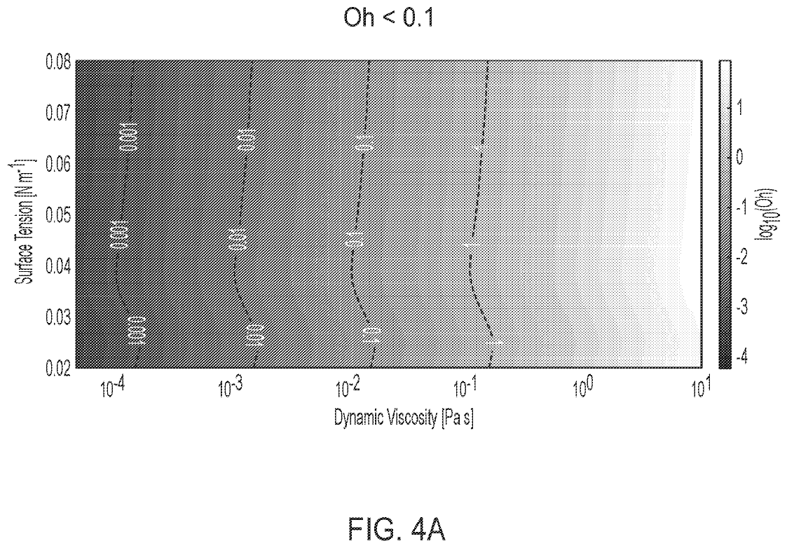

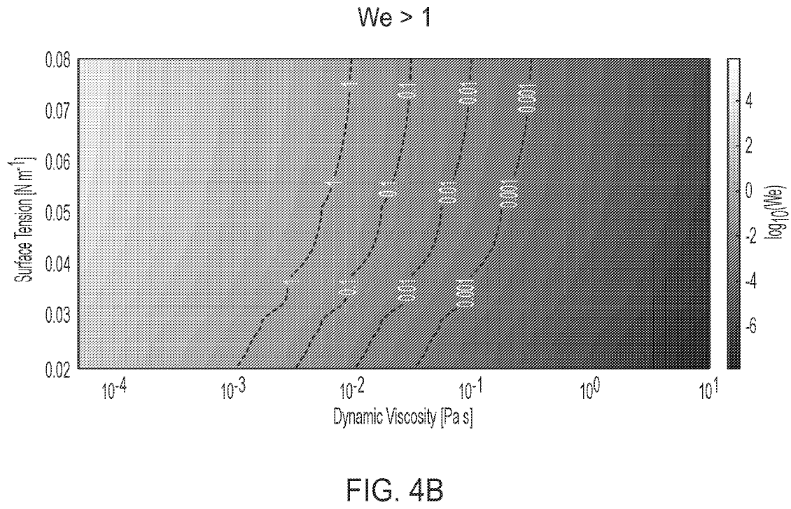

We is Weber number, and Re is Reynolds number; μ is the dynamic viscosity, ρ is the density, σ is the surface tension, and l is the characteristic length scale. The phase plots of Ohnesorge number and Weber number in the surface tension—dynamic viscosity space for a unidirectional conduit design are shown in the FIGS. 4A and 4B, respectively for Oh and We using MATLAB simulations to compute the velocity profile (the local velocity at various points within the tube) of the fluid and viscosity that is being transported through optimized curved tubes, which will then be used to compute the dimensionless numbers such as Re, Oh, and We. Additionally, an efficient transport is also realized when the viscous drag forces acting on the transporting fluid are minimized when compared to the inertial forces. Ohnesorge number lower than 0.1 indicates a lower energy loss due to fluid wall friction. To achieve an optimal transport volumetric flow rate, a combination of both Weber number greater than 1 and Ohnesorge number lower than 0.1 are necessary. In certain embodiments, the fluid for DF and/or TF may have an Ohnesorge number greater than 0.1 (or −1 in log₁₀ scale). This can be remedied by additive agents and medium that act to modify viscosity of DF and/or TF to maintain optimal transport properties scale). This can be remedied by additive agents and medium that act to modify viscosity of DF and/or TF to maintain optimal transport properties scale). This can be remedied by additive agents and medium that act to modify viscosity of DF and/or TF to maintain optimal transport properties scale). This can be remedied by additive agents and medium that act to modify viscosity of DF and/or TF to maintain optimal transport properties.

### I. Conduit

### A. Shape of the Conduit

In some embodiments, a conduit can be in the form of a tube, a shunt, a stent, a block, a wick, a channel, a cable, a hose, a pipe, a catheter, a spiral, a helix, a cone, an oval, a duplex shape having two separate concentric transport channels from the proximal and distal ends, a triplex shape having three separate concentric transport channels from the proximal and distal ends, or in a form of conduit with one or more flanges, valves, tabs, or wires, a shell or removable parts, that may be self-supporting or supported externally, bendable or rigid, or shape retaining or deformable. It may contain one or more nano-, micro-, or millimeter-sized pores.

### B. Materials of the Conduit

In certain embodiments, the conduit is formed of a base polymer consisting of non-biodegradable or biodegradable compounds. One class of composition which may be used involves non-biodegradable compounds, such as a polyurethane, a polyurethane copolymer, a fluoropolymer and a polyolefin or a silicone rubber. Another class of compositions which may be used involves biodegradable compounds (e.g., organic polymers): hydrophobic polyanhydrides, polyorthoesters, polyphosphazenes, polyphosphoesters/phosphoesters, and pseudopolyamino acids, poly(ethylene oxide), PEO, and poly(butylene terephthalate), PBT, that can degrade through surface erosion, diffusion, or hydrolysis or enzymatically, to produce biocompatible, toxicologically safe by-products that are further are eliminated by the normal metabolic pathways. Biodegradability can be engineered into polymers by the appropriate addition of chemical linkages such as anhydride, ester or amide bonds, among others. The degradation is affected by hydrolysis or enzymatic cleavage, resulting in a scission of the polymer backbone. Preferred are biodegradable polymers with hydrolysable chemical bonds. Wetting agents, emulsifiers and lubricants, such as sodium lauryl sulfate, calcium, and magnesium stearate, coloring agents, release agents, coating agents, sweetening, flavoring and perfuming agents, preservatives and antioxidants can also be present in the composition. Other suitable polymers include: poloxamers, polyamides, polyimides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene polyethylene glycol), poly(ethylene oxide), poly(ethylene terephthalate), poly(vinyl alcohols), poly(vinyl acetate), poly vinyl chloride polystyrene, polyvinylpryrrolidone, alginate, poly(caprolactone), dextran and chitosan, poly(lactic-co-glycolic acid) or PLGA, polycaprolactone or PLC, Gelatin, DNA hydrogen, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine).

The non-biodegradable polymer can be selected from, but not limited to, poly(ethylene vinyl acetate), poly(vinyl acetate), silicone polymers, polyurethanes, polyamides, polyimides, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, and copolymers and blends thereof. The metal can be selected from, but not limited to, cobalt, chromium, gold, nickel, platinum, stainless steel, titanium, tantalum, nickel-titanium, and alloys and combinations thereof.

In certain embodiments, the conduit material can be formed from a group of materials known as “hydrogels”, with the representative compounds which include but are not limited to polyvinyl alcohol, polyvinyl pyrrolidone, polyethylene oxide, and polyhydroxyethyl methacrylate alone or in combination. Other compositions include hydroxypropylmethyl cellulose and hydroxyethyl cellulose that can be broken-down by enzymes within the body, or polyesteramides, polyglycolic acid, polyvinyl alcohol, copolymers of polyethylene oxide/polylactic acid, and copolymers of glycolide/lactide, poly-L-lactic acid compounds, polycarbophil, polyvinyl pyrrolidone, polyvinyl alcohol, hydroxyethyl cellulose, hydroxypropylmethyl cellulose, and the like.

Another class of compositions which may be used as the conduit involves gels and polymers, including stimuli-responsive functionality, including but not limited to thermoresponsive gels, which include PNIPAAm, its copolymers with hydrophobic or hydrophilic comonomers, including but not limited to a copolymer of n-isopropylacrylamide and at least one acrylic and/or methacrylic monomer such as an alkyl acrylate (e.g., methyl acrylate, ethyl acrylate, butyl acrylate or 2-ethylhexyl acrylate); an acrylamide; or an acrylic acid or salt (e.g., 2-ethylacrylic acid, 2-propylacrylic acid, N-acryloxysuccinimide). Illustrative methacrylic monomers include a methacrylate (e.g., 2-hydroxymethacrylate, hydroxyethyl methacrylate, butyl methacrylate, methyl ether methacrylate or methyl methacrylate); a methacrylamide; or a methacrylic acid or salt. In certain embodiments, the acrylate monomer or methacrylate monomer may be modified with poly(ethylene glycol) to provide a co-poly(ethylene glycol) acrylate or co-poly(ethylene glycol) methacrylate prior to reaction with the n-isopropylacrylamide monomer.

In some embodiments, the conduit material can be a material that can attach to mucosa (including the interior side wall of the round window niche) for variable amounts of time up to about 1-2 hours in duration or more. Other patch structures of interest may be constructed from bilayer mucoadhesive polymers consisting of a “fast release” layer and a “sustained release” layer in order to achieve controlled drug release over a 24-hour period or longer.

In some embodiments, the polymers that can be used for forming the conduit include biostable or bioabsorbable polymers. Non-limiting examples include isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyimides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, alginates, gelatins, chitins, and combinations thereof.

In certain embodiments, the conduits can also be made with non-polymers. Non-limiting examples of useful non-polymers include sterols such as cholesterol, stigmasterol, β-sitosterol, and estradiol; cholesteryl esters such as cholesteryl stearate; C12-C24 fatty acids such as lauric acid, myristic acid, palmitic acid, stearic acid, arachidic acid, behenic acid, and lignoceric acid; C18-C36 mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C16-C18 fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Particularly useful non-polymers include cholesterol, glyceryl monostearate, glycerol tristearate, stearic acid, stearic anhydride, glyceryl monooleate, glyceryl monolinoleate, acetylated monoglycerides, and combinations thereof.

In some embodiments, the materials for the conduit designs can be selected from a group consisting of the Food and Drug Administration (FDA)-approved materials, such as silicones and fluoroplastics, Nylon, polyethylene terephthalate, Polycarbonate, Acrylonitrile Butadiene Styrene, Poly(p-phenylene oxide), Polybutylene terephthalate, Acetal, Polypropylene, Polyurethane, Polyetheretherketone, hydroxylpatite, Ultra-high molecular weight polyethylene, High Density Polyethylene, Low Density Polyethylene, Polystyrene High Impact, Polysulfone, Polyvinylidene fluoride, polystyrene, polymethylmethacrylate, latex, polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, and mixtures thereof. In addition, they can include polyelectrolyte hydrogels: ionic (including anionic or cationic) and ampholytic (including both anionic and cationic), for which incorporating more hydrophilic or hydrophobic monomers in hydrogel composition would allow for regulation of the volume transition behavior of the hydrogel. Non-limiting examples include hydrogel-forming materials such acrylate, polyacrylate, methacrylic acid, (dimethylamino)ethyl methacrylate, hydroxyethyl methacrylate, poly(vinyl alcohol)/poly(acrylic acid), 2-acrylamido-2-methylpropane sulfonic acid, [(methacrylamido)-propyl]trimethyl ammonium chloride, poly(N-vinyl-2-pyrrolidone/itaconic acid). Another category of materials can be represented by nonionic hydrogels. Non-limiting examples include poly(ethylene glycol), ethylene glycol diacrylate, polyethylene glycol diacrylate poly(ethylene oxide), diacrylate, acrylamide, polyacrylamide, methylenebisacrylamide, N-isopropylacrylamides, poly(vinyl alcohol) and mixtures thereof. In some embodiments, the hydrogel can be made of natural materials, such as proteins (e.g., collagen and silk) and polysaccharides (e.g., chitosan, dextran and alginate), superparamagnetic iron oxide nanoparticles (SPION), cubosomes, polymerosomes, and combinations thereof. In some embodiments, the conduits can be made of metals or metal oxides.

In certain embodiments, the materials can also contain colloidal particles that are dispersed or suspended in another substance. Non-limiting examples of suitable colloidal particles that can be used in the hydrogel-based conduits include polystyrene and polymethylmethacrylate, melamine resins (having a large number of reactive amino and imino groups for immobilization of different metal ions or metal nanoparticles), silica and polydivinylbenzene microparticles. In some embodiments the colloidal particles are made of one or more of the following polymers: poly(methyl methacrylate), polyacrylate, polyalkylacrylate, substituted polyalkylacrylate, polystyrene, poly(divinylbenzene), polyvinylpyrrolidone, poly(vinylalcohol), polyacrylamide, poly(ethylene oxide), polyvinylchloride, polyvinylidene fluoride, polytetrafluoroethylene, other halogenated polymers, hydrogels, organogels, or combinations thereof. Other polymers of different architectures can be utilized as well, such as random and block copolymers, branched, star and dendritic polymers, and supramolecular polymers. In certain embodiments, the colloidal particles are of natural origin (biopolymer colloid), such as a protein- or polysaccharide-based material, silk fibroin, chitin, shellac, cellulose, chitosan, alginate, gelatin, or a mixture thereof. In certain embodiments, the colloidal particles include one or more metals, such as gold, palladium, platinum, silver, copper, rhodium, ruthenium, rhenium, titanium, osmium, iridium, iron, cobalt, or nickel, or a combination thereof. In certain embodiments, the colloidal particles include one or more oxides, such as silica, alumina, beryllia, noble metal oxides, platinum group metal oxides, titania, tin oxide, zirconia, hafnia, molybdenum oxide, tungsten oxide, rhenium oxide, vanadium oxide, tantalum oxide, niobium oxide, chromium oxide, scandium oxide, yttria, lanthanum oxide, ceria, thorium oxide, uranium oxide, other rare earth oxides, or a combination thereof. Other class of particles to include is ferromagnetic, ferrimagnetic or superparamagnetic particles (diameter usually 10 nanometers or less). Exemplary nanoparticles include iron, nickel and cobalt containing particles, such as magnetite or hematite, Colloidal particles useful in the conduits described herein can be charged, or uncharged, hydrophilic, hydrophobic, or amphiphilic. In some embodiments, the conduits can contain colloidal particles of two or more different types, compositions, or average sizes.

In any of these preceding embodiments, the precursor composition can comprise one or more additives selected from the group consisting small molecules, dispersed liquid droplets, or microparticle and nanoparticle fillers (e.g., talc, calcium carbonate, calcium phosphate), anti-oxidants, UV stabilizers, plasticizers, anti-static agents, porogens, slip agents, processing aids, foaming or antifoaming agents, nucleating agents and fillers to enhance mechanical properties or roughness, and to control optical properties or viscosity and uniformity of application, according to certain embodiments.

Other excipients can be found in the United States Pharmacopeia and National Formulary and FDA's database Drug Information: Electronic Orange Book.

In certain embodiments, for medical and non-medical fluidic applications, the materials for the conduit designs listed in this innovation can include metals selected from the group of Li, Be, B, Na, Mg, Al, Si, K, Ca, Sc, Ti, V, Cr, Mn, Fe, Co, Ni, Cu, Zn, Ga, Ge, Rb, Sr, Y, Zr, Nb, Mo, Ru, Rh, Pd, Ag, Cd, In, Sn, Sb, Cs, Ba, Hf, Ta, W, Re, Os, Ir, Pt, Au, Pb, Bi, La, Ce, Pr, Nd, Sm, Eu, Gd, Tb, Dy, Ho, Er, Tm, Yb, Lu, and their oxides or a combination thereof. In certain embodiments, the metal-containing conduit contains aluminum and the roughened metal-containing surface contains boehmite. In certain embodiments, the metal-containing sol-gel precursor contains a porogen.

In certain embodiments, the materials for the conduit designs can include metal foams or porous metallic substrates. In certain embodiments, these porous substrates can be formed typically by the solidification process of a mixture of pre-melted metals with injected gas/gas-releasing blowing agents, or by compressing metal powders into special tooling to form different shapes and forms (e.g., sheet, cylindrical shape, hollow cylinders etc.). Metal foams can be manufactured either in closed-cell or open-cell structures (i.e., interconnected network of metals). Metal foams of different materials, such as aluminum, titanium, nickel, zinc, copper, steel, iron, or other metals and alloys, can be used, and have been produced by various methods, such as direct foaming and powder compact melting methods, which have been extensively discussed in J. Banhart, Prog. Mater. Sci 46, 559-632 (2001), which is incorporated herein by reference.

### C. Lubricating Liquids

Lubricating liquid can be selected from a number of different fluids. These fluids can be selected based on their suitability for biocompatibility, low toxicity, anti-fouling performance, drug release and chemical stability under physiological conditions. In one or more aspects, the lubricating liquid is a chemically inert, high-density biocompatible fluid, non-limiting examples of which include castor oil, silicone oils, fluocinolone acetonide oil, mineral oil, fully or partially fluorinated hydrocarbons and other partially or fully fluorinated compounds bearing heteroatom-containing functional groups, organosilicone compound, silicones, tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, fully, partially or non-fluorinated poly- and oligoethers, fully, partially or non-fluorinated poly- and oligoesters, fully, partially or non-fluorinated poly- and oligoisocyanates, lecithin, glycerin, lipid emulsions, peanut oil, cottonseed oil, safflower oil, sesame oil, olive oil, corn oil and soybean oil, almond oil, borage oil, black currant seed oil, castor oil, corn oil, safflower oil, rapeseed oil, coconut oil, palm oil, canola oil, tea tree oil, and combinations thereof.

The lubricating liquid infiltrates, wets, and stably adheres to the substrate. Moreover, it is chemically inert with respect to the solid substrate and the fluid to be repelled. The lubricating liquid is non-toxic. Further, the lubricating liquid in accordance with certain aspects is capable of repelling immiscible fluids of any surface tension. In one or more aspects, the lubricating liquid is a chemically-inert and high-density biocompatible fluid. Further, the lubricating liquid is capable of repelling immiscible fluids, and in particular biological fluids of any surface tension. For example, the enthalpy of mixing between the fluid to be repelled and lubricating liquids be can be sufficiently high (e.g., water and oil) that they phase separate from each other when mixed together. In one or more embodiments, lubricating liquid is inert with respect to the solid surface and biological fluid. Lubricating liquid flows readily into the recesses of the roughened surface and generally possesses the ability to form an ultra-smooth surface when provided over the roughened surface. Some exemplary suitable lubricating liquid includes perfluorinated hydrocarbons, organosilicone compound (e.g., silicone elastomer), hydrophobic materials, and the like. In particular, the tertiary perfluoroalkylamines (such as perfluorotri-npentylamine, FC-70 by 3M, perfluorotri-n-butylamine FC-40, etc), perfluoroalkylsulfides and perfluoroalkylsulfoxides, perfluoroalkylethers, perfluorocycloethers (like FC-77) and perfluoropolyethers (such as KRYTOX family of lubricants by DuPont), perfluoroalkylphosphines and perfluoroallcylphosphineoxides, fully, partially or non-fluorinated poly- and oligoethers, fully, partially or non-fluorinated poly- and oligoesters, fully, partially or non-fluorinated poly- and oligoisocyanates, as well as their mixtures can be used for these applications, as well as their mixtures with perfluorocarbons and any and all members of the classes mentioned. In addition, long-chain perfluorinated carboxylic acids (e.g., perfluorooctadecanoic acid and other homologues), fluorinated phosphonic and sulfonic acids, fluorinated silanes, and combinations thereof can be used as the lubricating liquid. The perfluoroalkyl group in these compounds could be linear or branched and some or all linear and branched groups can be only partially fluorinated. In certain embodiments, hydrophobic materials such as olive oil, silicone oil, hydrocarbons, and the like can be utilized as the lubricating liquid. In certain embodiments, ionic liquids can be utilized as the lubricating liquid.

In certain embodiments, the lubricating liquids used to facilitate repellency are selected to create a fluid surface that is intrinsically smooth, stable, and defect free. The lubricating liquid of certain embodiments infiltrate, wet, and stably adhere to the substrate. Moreover, the lubricating liquid of certain embodiments should be chemically inert with respect to the solid substrate and the fluid to be repelled. The lubricating liquid of certain embodiments should provide for adequate release of the drug and be non-toxic. Further, the lubricating liquid in accordance with certain aspects is capable of repelling immiscible fluids of any surface tension. In one or more aspects, the lubricating liquid is a chemically-inert and high-density biocompatible fluid.

Lubricating liquid can be selected from a number of different fluids according to certain embodiments. These fluids can be selected based on their suitability for drug release, biocompatibility, low toxicity, anti-clotting performance, and chemical stability under physiological conditions. In one or more aspects, the lubricating liquid is a chemically inert, high-density biocompatible fluid, non-limiting examples of which include vegetable oils. Vegetable oil refers to oil derived from plant seeds or nuts. Exemplary vegetable oils include, but are not limited to, almond oil, borage oil, black currant seed oil, castor oil, corn oil, safflower oil, soybean oil, sesame oil, cottonseed oil, peanut oil, olive oil, rapeseed oil, coconut oil, palm oil, canola oil, tea tree oil, etc. Vegetable oils are typically “long-chain triglycerides,” formed when three fatty acids (usually about 14 to about 22 carbons in length, with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of oil-in-water emulsions. In certain embodiments, hydrogenated vegetable oils, which are produced by controlled hydrogenation of the vegetable oil, can be used in the systems disclosed herein.

Other oils can also be used but it can be necessary to modify the composition to provide for adequate solubilization of the drug in the oil. For example, perfluorinated hydrocarbons or organosilicone compound (e.g., silicone elastomer) and the like can be utilized. In particular, in certain embodiments the tertiary perfluoroalkylamines (such as perfluorotri-n-pentylamine, FC-70 by 3M, perfluorotri-n-butylamine FC-40, etc), perfluoroalkylsulfides and perfluoroalkylsulfoxides, perfluoroalkylethers, perfluorocycloethers (like FC-77) and perfluoropolyethers (such as KRYTOX family of lubricants by DuPont), perfluoroalkylphosphines and perfluoroalkylphosphineoxides, fully, partially or non-fluorinated poly- and oligoethers, fully, partially or non-fluorinated poly- and oligoesters, fully, partially or non-fluorinated poly- and oligoisocyanates, as well as their mixtures can be used for these applications, as well as their mixtures with perfluorocarbons and any and all members of the classes mentioned. In addition, long-chain perfluorinated carboxylic acids (e.g., perfluorooctadecanoic acid and other homologues), fluorinated phosphonic and sulfonic acids, fluorinated silanes, and combinations thereof can be used as lubricants in certain embodiments. The perfluoroalkyl group in these compounds could be linear or branched and some or all linear and branched groups can be only partially fluorinated in certain embodiments. To improve drug solubility in these other oils, surfactants and emulsifiers can be included in the compositions in certain embodiments.

For applications in certain non-medical applications, the lubricant can be selected from the group consisting of fluorinated lubricants (liquids or oils), silicones, mineral oil, plant oil, water (or aqueous solutions including physiologically compatible solutions), ionic liquids, polyolefins, including polyalpha-olefins (PAO), synthetic esters, polyalkylene glycols (PAG), phosphate esters, alkylated naphthalenes (AN) and silicate esters or any mixture thereof.

In certain embodiments, the lubricant has a high density. For example, lubricant that has a density that is more than 1.0 g/cm³, 1.6 g/cm³, or even 1.9 g/cm³ can be used.

In certain embodiments, the lubricant has a low freezing temperature, such as below than −5° C., −25° C., or even below than −80° C. Having a low freezing temperature will allow the lubricant to maintain its slippery behavior at reduced temperatures and to repel a variety of liquids or solidified fluids.

In certain embodiments, the lubricant can have a low depletion rate from the surface through evaporation, such as less than 1 nm/s, less than 0.1 nm/s, or even less than 0.01 nm/s. Taking a typical thickness of lubricant to be about 10 μm and an evaporation rate of about 0.01 nm/s, the surface can remain highly liquid-repellant for a long period of time without any refilling mechanisms.

In certain embodiments, the viscosity of the oil is in the range of about 1 to 2000 cSt. In certain embodiments, the viscosity of the oil is in the range of about 1 to 500 sCt.

In certain embodiments, the viscosity of the oil is in the range of about 8 to 1500 cSt. In certain embodiments, the viscosity of the oil is in the range of about 10 to 550 cSt. In certain embodiments, the viscosity of the oil is in the range of about 8 to 80 cSt. In certain embodiments, the viscosity of the oil is in the range of about 8 to 350 cSt. In certain embodiments, the viscosity of the oil is in the range of about 80 to 350 cSt. In certain embodiments, the viscosity of the oil is in the range of about 80 to 550 cSt.

### D. Stimuli-Responsive Materials

The stimuli-responsive valves for the conduit lumen or the conduits themselves can comprise a nematic, smectic, chiral, discotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

In one embodiment, the shape-responsive layer comprises a liquid crystal elastomer. Shape-changes in monodomain LCEs, which have a uniformly aligned liquid crystal (LC) director, can range from 10% to 400% of the initial LCE size. In some embodiments, the LCE is a polydomain liquid crystal elastomer. In some embodiments, the LCE includes a nematic director and a mesogen (liquid crystal molecule or moiety) associated with a polymer. In some embodiments, the mesogen content of the LCE ranges from about 20% molar content to about 90% molar content of the liquid crystal elastomer. In some embodiments, the mesogen is generally a molecule that produces a liquid crystal phase at room temperature and can include at least one of aromatic rings, aliphatic rings, poly aromatic rings, poly aliphatic rings, phenyls, biphenyls, cyanobiphenyls, benzenes, and combinations thereof. In some embodiments, the mesogen is functionalized with one or more functional groups, such as alkenes, alkanes, alkynes, carboxyl groups, esters, halogens, and combinations thereof. In certain embodiments, the mesogen is 4-methoxyphenyl 4-(3-butenyloxy) benzoate.

In some embodiments, mesogens in LCEs are cross-linked polymers. In some embodiments, the polymer includes at least one of polysiloxanes, poly(methyl) siloxanes (PMS), poly(dimethyl) siloxanes (PDMS), polymethylhydrosiloxane (PMHS), poly(methyl methacrylate), polyethylene, polypropylene, poly(butylacrylate) network chains and combinations thereof.

The polymers can be associated with mesogens in various arrangements. For instance, in some embodiments, the mesogens can be cross-linked to polymers. The crosslinker can be any reactive molecule that produces a physically or chemically crosslinked, elastomeric network. For example, a di(methacrylate) crosslinker is used or a diacrylate crosslinker. The crosslinker concentration can be varied to increase or decrease the elastomer modulus, at higher or lower crosslinker contents, respectively. Various catalysts and methods can be used to crosslink the network, including thermal annealing or platinum catalysts providing the desired level of reactivity. The solvent content can also be varied during synthesis.

In some embodiments, a plurality of mesogens can be covalently coupled to a single polymer chain. In some embodiments, a plurality of mesogens can be covalently coupled to multiple polymer chains. In some embodiments, the mesogens and polymers can be intertwined within a matrix. LCEs can be made using methods known in the art.

In yet another embodiment, conductive material can be added to the shape-responsive layer. The conductive filler can provide the LCE nanocomposite with an electrical, magnetic, or light-induced response, as examples. For example, the LCE can comprise one or more wires. Alternatively or in addition to, carbon nanoconduits, carbon black nanoparticles, or conductive gold nanoparticles can be used.

In addition to tympanostomy conduits, the embodiments of the present disclosure can also enhance the field of other conduit-like medical implants, such as but not limited to surgical drains, vascular stents, catheter, dialysis tubing, feeding conduits, colostomy conduits, and eustachian implants, laryngeal stents, ventriculoperitoneal shunts.

In some embodiments, the simuli-responsive valves for the conduit lumen or the conduits themselves can comprise stimuli-responsive (or intelligent) hydrogels, including but not limited to temperature-, light-, magnetic-field, electric-field-, pH-, electrolyte-, glucose-ultrasonic-radiation-, calcium-, antigen-, DNA- and enzyme-responsive. For instance, in some embodiments, the hydrogels can include methylcellulose, hydroxypropyl methylcellulose, chitosan, isopropylacrylamide (NIPAAm), poly(vinyl methyl ether) (PVME), poly(N-vinylisobutyramide) (PNVIBA), poly(ethylene oxide-propylene oxide-b-ethylene oxide) (PEO-PPOPEO), poly(N-isopropylacrylamide) (PNIPAAm); polyvinyl sulfonic acid, polymethacrylic acid, polydiethylaminoethyl methacrylate, polydimethylaminoethyl methacrylate, ethylene-co-vinyl alcohol, Ethylene-co-vinyl acetate (EVAc), poloxamer and alginic acid, hyaluronic acid, cellulose esters, N-vinyl pyrrolidone, hydroxy alkyl methacrylate, acrylamide and N—N-dialkylacrylamide, polysaccharide-based hydrogel, catechol borate ester, Pluronic, and others.

In some embodiments, the stimuli-responsive valves for the conduit lumen or the conduits themselves can comprise shape-memory polymers in this category are polyurethanes polyurethanes with ionic or mesogenic components, block copolymers of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran; polynorbornene, or organic-inorganic hybrid polymers consisting of polynorbornene units that are partially substituted by polyhedral oligosilsesquioxane (POSS); copolymers consisting of polycyclooctene (PCOE) and poly(5-norbornene-exo,exo-2,3-dicarboxylic anhydride) (PNBEDCA), synthesized through ring-opening metathesis polymerization (ROMP), also modified by grafting reaction of NBEDCA units with polyhedral oligomeric silsesquioxanes (POSS) to afford a functionalized copolymer P(COE-co-NBEDCA-g-POSS), polycaprolactone, polyenes, nylons, blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, and blends thereof. In some embodiments, the polymeric material includes a natural polymer, e.g., zein, casein, gelatin, gluten, serum albumin, collagen, polysaccharides, polyhyaluronic acid, poly(3-hydroxyalkanoate)s, alginate, dextran, cellulose, collagen or mixtures of these polymers. In some embodiments, the polymeric material includes a synthetic polymer, e.g., chemical derivatives of collagen, chemical derivatives of cellulose, polyphosphazenes, poly(vinyl alcohols), polyamides, polyimides, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, degradable polymers, polyester amides, polyanhydrides, polycarbonates, polyorthoesters, polylactides, polyglycolides, polysiloxanes, polyurethanes, cellulose derivatives or mixtures of these polymers. In some embodiments, polymeric material includes mixtures of natural and synthetic polymers. In some embodiments, the polymeric material is cross-linked.

### II. Plugs

As explained above, a plug can be provided into the conduit. Some exemplary shapes and materials for the plugs are described below.

### A. Shape of Plugs

The plug may be in a number of different forms, such as a valve, a membrane, a film, a lid, a handle, an inclusion, a pellet, a cage, a capsule, a powder, a foam, a gel, and the like.

In some embodiments, the plug can have an extension into the middle ear in form of a sponge, absorbing pad, foam, receptacle, mesh, porous cladding, inflatable balloons, catheter, fiber, sieve, uniformly or non-uniformly expandable gel, roll, swirl, ball, wrap, film, and dressing.

### B. Materials of Plugs

In some embodiments, the valve is selected from one of a stimuli-responsive polymer, a gas-selective mobile membrane, stimuli-responsive cilia-like and hair-like fibers, platelets, pillars, reconfigurable tunable nano- or microstructures with functionalized tips or functionalized pillars, and combinations thereof.

The stimuli-responsive plugs for the conduit lumen can comprise a nematic, smectic, chiral, discotic, bowlic liquid crystals with thermotropic, lyotropic and metallotropic phases. Liquid crystal can also be a cholesteric (chiral nematic) liquid crystal, a smectic A, smectic C, or smectic C* (chiral smectic C), a ferroelectric or antiferroelectric smectic liquid crystal, a liquid crystal compound comprising a bent-core molecule, a columnar mesophase liquid crystal, a discotic liquid crystalline porphyrin, or a lyotropic liquid crystal, or any combination thereof. Next example would be a photo-responsive liquid crystal composition composed of a liquid crystalline compound and a gelling agent mixed with the liquid crystalline compound to form a gelling mixture, wherein the liquid crystalline compound is capable of being controlled in a state oriented in one direction by an irradiation of light. As the specific liquid crystalline compound, can be used those exhibiting a nematic phase at room temperature such as, cyanobiphenyl compounds, phenylcyclohexane compounds, benzylideneaniline compounds, phenylbenzoate compounds, phenylacetylene compounds and phenylpyrimidine, cyanobiphenyl compounds such as 4-pentyl-4′-cyanobiphenyl, benzylideneaniline compounds such as 4-methoxybenzylidene-4′-butylaniline, phenylcyclohexane compounds such as 4-(trans-4-pentylcyclohexyl)benzonitrile. In addition, isoleucine derivatives having an azobenzene structural part, BDH-17886 from Merck Ltd., liquid crystal composition p-meth-oxy-n-p-benzilidene butylaniline (MBBA) can be used. Liquid crystal mixtures with polymers can include polyurethane (PU), polyethylene oxide (PEO), polyacrylonitrile (PAN), polyvinyl acetate (PVA), cellulose acetate; polyaniline, polypyrrole, polythiophene, polyphenol, polyacteylene, polyphenylene, poly(lactic acid) (PLA), poly(methyl methacrylate) (PMMA), poly(glycolic acid) (PGA), poly(ethylene oxide), polyacrylate, polyester, polyamide, polyolefin, polyvinylchloride (PVC), poly(amic acid), polyimide, polyether, polysulfone, and any combination thereof.

The plug materials can also be selected from, but not limited to, poly(lactic-co-glycolic acid) or PLGA, polycaprolactone or PLC, Chitosan, Gelatin, DNA hydrogen, acetalated dextran, poly(lactide), poly(glycolide), poly(lactide-co-glycolide), poly(lactic acid), poly(glycolic acid), poly(lactic acid-co-glycolic acid), poly(lactide)/poly(ethylene glycol) copolymers, poly(glycolide)/poly(ethylene glycol) copolymer, poly(lactide-co-glycolide)/poly(ethylene glycol) copolymers, poly(lactic acid)/poly(ethylene glycol) copolymer, poly(glycolic acid)/poly(ethylene glycol) copolymer, poly(lactic acid-co-glycolic acid)/poly(ethylene glycol) copolymer, poly(caprolactone), poly(caprolactone)/poly(ethylene glycol) copolymer, poly(orthoester), poly(phosphazene), poly(hydroxybutyrate), poly(hydroxybutyrate), poly(lactide-co-caprolactone), polycarbonate, polyesteramide, polyanhydride, poly(dioxanone), poly(alkylene alkylate), polyethylene glycol/polyorthoester copolymer, polyurethane, poly(amino acid), polyetherester, polyacetal, polycyanoacrylate, poly(oxyethylene)/poly(oxypropylene) copolymer, Sephadex® copolymers (made from dextran cross-linked with epicholorhydine).

In some embodiment, the plug or the conduit itself can represent or comprise a rigid or flexible absorbent or thread that has a mechanism of on-demand guiding and releasing the drug formulation to a site of interest within the middle ear either passively, under stimulus, or upon introduction of a drug formulation. Such thread would allow a precise delivery of the drug formulation, for example, to the round window membrane. The conduit with the thread can change shape, orientation, size, porosity on demand upon introduction of a therapeutic, activating, priming, or reversal agents. The conduit can also be created with materials that have an absorbent-like-property (i.e. created from fibers directed into the middle ear) to enhance the flowrate of the drug formulation.

In some embodiments the tube conduit can comprise pores, e.g., from 5% to about 10%, from about 10% to about 20%, from about 20% to about 30%, from about 30% to about 40%, from about 40% to about 50%, from about 50% to about 60%, from about 60% to about 70%, from about 70% to about 80%, or from about 80% to about 90%.

The wick can be made of a single material, or can comprise two or more materials layered upon one another. Exemplary materials include, but are not necessarily limited to, biocompatible polymers, bioerodable materials, elastomers, metals, metal alloys, glasses, laminates of hydrophilic polymers and hydrophobic polymers, multilaminates or polymer, metals, and/or glasses; and the like; biocompatible polymeric materials including but are not necessarily limited to, homopolymers and copolymers of vinyl acetate (e.g., ethylene vinyl acetate copolymer); homopolymers and copolymers of acrylates (e.g., poly(methyl) methacrylate (PMMA), polyethylmethacrylate, ethylene glycol dimethacrylate, ethylene dimethacrylate and hydroxymethyl methacrylate); polyurethanes; polyethylenes; polyvinylchlorides; polycarbonates; polyamides; polysulfones; polyesters; polyimides; halogenated polymers (e.g., polytetrafluoroethylene (PTFE), polyvinyl fluoride, polychlorotrifluoroethylene, copolymers tetrafiuoroethylene and hexafluoropropylene; PFA, and the like); polyolefins (e.g., high density polyethylene (HDPE), low density polyethylene (LDPE), linear low density polyethylene (LLDPE), polypropylenes, and the like); polystyrenes; nylons; urethanes; homopolymers and copolymers of acrylonitrile (e.g., acrylonitrile-butadiene-styrene polymer, styrene acrylonitrile, polycarbonate-acrylonitrile-butadiene-styrene; and the like); polyvinylpyrrolidone; 2-pyrrolidone; polyacrylonitrile butadiene; cellulose acetate; polyethylene terephthalate; polymethylpentene; polyisobutylene; polymethylstyrene; polyvinylidine chloride and homopolymers and copolymers of polyvinylidine chloride (e.g., polyvinylchloride-acrylic copolymers); PEBAX™; HYTREL™; and other similar compounds known to those skilled in the art. Further exemplary polymers are described in Brydson's Plastics Materials, Eighth Edition, 2016. Suitable, biocompatible elastomers include, but are not necessarily limited to, biocompatible elastomers such as medical grade synthetic (e.g., silicone) rubbers; hydrogels, polyvinyl chloride elastomers; polyolefins; homopolymeric and copolymeric elastomers: urethane-based elastomers; natural rubbers; and fluorinated polymers (e.g., PTFE), and the like. Metallic materials suitable for the delivery body comprise stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; titanium nitride-coated stainless steel, titanium, platinum, tantalum, gold, and their alloys; TEFLON™; nickel titanium; and superelastic nickel titanium.

In some embodiments, the plug can be solubilized by solvents present in the tube. For example, the plug can be composed of (but not limited to) FDA-approved commercially available salts containing anions such as acetate, iodide, benzenesulfonate, isethionate, benzoate, lactate, bicarbonate, lactobionate, bitartrate, malate, bromide, maleate, calcium edetate, Mandelate, Carbonate, Methylbromide, Chloride, Methylnitrate, Dihydrochloride, Mucate, Edetate, Napsylate, Edisylate, Nitrate, Esylate, Pantothenate, Fumarate, Phosphateldiphosphate, Gluceptate, Polygalacturonate, Gluconate, Salicylate, Glutamate, Stearate, Glycollylarsanilateg, Subacetate, Hexylresorcinate, Succinate Hydrabamine, Sulfate, Hydrobromide, Tannate, Hydroxynaphthoate, Teoclate, Triethiodide, or cationis such as Benzathid, Aluminum, chloroprocaine, Calcium, Choline, Lithium, Diethanolamine, Magnesium, Ethylenediamine, Potassium, Meglumine, Sodium, Procaine, Zinc.

### III. Agents of the Drug Formulation, Therapeutic Formulation and Plug Formulation

As explained above, the drug formulation, therapeutic formulation (i.e., tube formulation), and plug formulation can each have one or more of the following agents: a therapeutic agent, a priming agent, an activating agent and a reversal agent. Some exemplary materials for these different agents are discussed below.

### A. Therapeutic Agent

As described herein, the therapeutic agent refers to a compound, drug, pharmaceutical composition, or combinations thereof, that is effective for the treatment of ear and hearing disorders. A therapeutic agent may comprise one or more agents (drugs). Pro- and co-drugs in the present application may include one or more drugs combined. Co-drugs in the present application also include co-drug of a single compound (i.e, a co-drug in which the two active components are the same agent). Those skilled in the art will readily appreciate that the present application is not limited to the specific agents listed herein, but extends to compounds with desirable therapeutic effects and/or for which the use is indicated for the particular disease state of interest.

In certain embodiments, the therapeutic agents may be selected from the group consisting of anti-inflammatory, anti-allergic, and analgesic agents, non-steroidal anti-inflammatory (NSAID) agents, tranquilizing agents, corticosteroids, volume expanding agents, vasodilating agents, antihistaminic agents, anticholinergic agents, vaccines, adjuvants, enzymes, monoclonal antibodies, drug dissolution and disintegration promoters, exosomes, microcrystals, solid lipid nanoparticles, preservatives, antifoams, taste-masking agents, chelating agents, buffering agents, bulking agents, anti-coagulants, antiviral agents, immunosuppressive agents, antacids and H2-blockers, antiemetics, calcium channel blockers, anticancer agents, vitamins, silk, vascular rheologic agents, neuroprotective agents, neuromodulators; anti-apoptotic agents; antiseptics, therapeutic peptides, therapeutic polysaccharides, hormones, free radical scavengers, neurotrophins, fungicides, antibodies, antigens, bacteriocides, urea, mannitol, sorbitol, glycerol, lidocaine, xylocaine, epinephrine, immunoglobulins, sodium chloride, heparin, hyaluronidase, aminoglycoside neurotrophins, cortico-steroids, vitamins, cortisone, analgesics, antioxidants, neurotrophins, anti-apoptotic agents, anti-necrotic agents, leupeptin or combinations thereof.

The composition can be formulated as a frozen composition, e.g., flash frozen, dried or lyophilized for storage and/or transport. In addition, the composition can be administered alone or in combination with a carrier, such as a pharmaceutically acceptable carrier or a biocompatible scaffold.

In certain embodiments, the therapeutic agents may be a pharmaceutical compound that is typically used to treat inner ear tissues including but are not limited to urea, mannitol, sorbitol, glycerol, lidocaine, xylocaine, epinephrine, immunoglobulins, sodium chloride, steroids, heparin, hyaluronidase, aminoglycoside antibiotics (streptomycin/gentamycin), antioxidants, neurotrophins, nerve growth factors, various therapeutic peptides, and polysaccharides, cortico-steroids, vitamins or rheologic agents, cortisone, analgesics, antipyrine, benzocaine, procaine, antioxidants, e.g., methionine, N-acetylcysteine, trolox, neurotrophins, e.g., GDNF or BDNF, anti-apoptotic or anti-necrotic agents, e.g., leupeptin, caspase inhibitors, Ginkgo biloba extract, etc. Of particular interest in this list are compounds which are used to alter the permeability of the round window membrane within the ear using, for example, hyaluronidase and iontophoretic techniques. For example, hyaluronic acid and histamine increase the permeability of the round window membrane and result in higher concentrations of the drug in the perilymph of the inner ear. Such improved permeation must come however without disturbing the osmotic balance between inner ear perilymph and the middle ear space and without inducing toxicity in the cochlea. Particular attention has to be paid to potential ototoxicity from permeability enhancing substances, which may themselves pass across the round window and have a cytotoxic effect within the inner ear.

In certain embodiments, the therapeutic agent treatments include the use of vasodilators, e.g., papaverine, histamine, nicotinic acid, procaine, and niacin; rheologic agents such as pentoxyfylline, heparin and warfarin; anti-inflammatory agents, particularly corticosteroids; antiviral agents such as acyclovir, famciclovir, valacyclovir and amantadine; and diatrizoate meglumine; anticoagulants; plasma expanders such as dextran; renograffin or urograffin, and growth factors such as insulin-like growth factor-1 (IGF-1) and/or fibroblast growth factor-2 (FGF-2), or their agonists. Additionally, one could use otoprotective agents, such as free radical scavengers, iron chelators and certain NMDA receptor antagonists, aspirin, reduced glutathione, N-methyl-(D)-glucaminedithiocarbamate, (D)-methionine, and iron chelators such as tartrate and maleate, and vasodilators such as betahistine, niacin, and papaverine.

In certain embodiments, the therapeutic agent may be an antibiotic including but not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, oat extract (avenanthramide), or combinations thereof. Particular antibiotics include neomycin B, kanamycin A, kanamycin B, erythromycin and azithromycin, vancomycin, gentamicin, amikacin), dibekacin, tobramycin, streptomycin, paromomycin, sisomicin, butirosin, geneticin, isepamicin, and netilmicin, gentamicins A, C1, C1, C2 and D; neomycins B and C, hybrimycin, propikacin (UK 31214), ribostamycin, seldomycin, trehalosamine, D-mannosyl-D-glucosaminide, apramycin, bluensomycin, netromycin, tobramycin, sisomicin, destomycin, Antibiotic A-396-I, dibekacin, kasugamycin, fortimicin, or derivatives, analogs or variants thereof and the like antimicrobial agents to provide improved methods for prevention and treatment diseases induced by Gram-positive, Gram-negative, and acid-fast bacteria. Antibiotics can be introduced with steroids, calcineurin inhibitors, interleukin inhibitors, TNF-α inhibitors, platelet activating factor antagonists, nitric oxide synthase inhibitors, histamine antagonists, estrogen-related receptor beta modulators aural pressure modulators, cyclosporin A (CsA) and CsA derivatives, voclosporin, FK506 (tacrolimus) and derivatives, ascomycin, pimecrolimus (SDZ AM 981, 33-epi-chlor-33desoxy-ascomycin), L-732,531 (32-O-(1-hydroxyethylindol-5-yl)-ascomycin), L-685,818 (FK506BD) and V-10,367; FMPP (4-(fluoromethyl)phenyl phosphate), tyrphostins, norcantharidin, okadaic acid, endothall, kaempferol, barbiturates, 1,5-dibenzoyloxymethyl-norcantharidin, gossypol, Lie120, PD144795, diberufin, dipyridamole, NCI3, INCA compounds, BTPs or 3,5-bis(trifluoromethyl)pyrazoles, BTP1, BTP2 (YM-58483), BTP3, BTP A-285222, ST1959, AM404, UR-1505, Triflusal, rocoglamide derivatives, WIN 53071, trifluoroperazine, KRM-III, caffeic acid phenyl ethyl ester (CAPE), YM-53792, quinazolinediones, pyrrolopyrimidinediones, NFAT-68, NFAT-133, punicalagin, imperatorin, quinolone alkaloids, 2-aminopyrimidine derivatives compound, pyrimidine derivatives compound, quinazoline derivatives compound, quinazoline sulfonamide compound, bicyclic heteroaryl-substituted imidazole compound, thieno-pyrimidine compound, furo-pyrimidine compound and mequitazine compound, 1-[(5-chloro-1H-indol-2-yl)carbonyl]-4-methylpiperazine or 4-((3R)-3-Aminopyrrolidin-1-yl)-6,7-dihydro-5H-benzo[6,7]cyclohepta[1,2-d]pyrimidin-2-ylamine or cis-4-(Piperazin-1-yl)-5,6,7a,8,9,10,11,11a-octahydrobenzofuro[2,3-h]quinazolin-2-amine or 7-(furan-2-yl)-4-(piperazin-1-yl)quinazolin-2-amine or 1-(7-(2-amino-6-(4-methylpiperazin-1-yl) pyrimidin-4-yl)-3,4-dihdroisoquinolin-2(1H)-yl)-2-cyclopentylethanone or 1-[(5-Chloro-1H-benzimidazol-2-yl)carbonyl]-4-methylpiperazine maleate or PF-3893787 or PF-3893787-18 or JNJ39758979 or UR-63325, antisense RNA or DNA molecules, small inhibitory RNAs (siRNAs), short hairpin RNA, micro RNA, DNAzymes, modified or synthetic DNA or RNA degradation-resistant polynucleoside amides, peptide nucleic acids (PNAs), locked nucleic acids (LNAs) and other nucleobase-containing polymers, 1H-pyrrole-2,5-dione compounds, 1,5-dihydro-2H-pyrrol-2-one compounds, 3-(pyridin-2-yl)-1H-indol-2-ol based compounds, 2-pyrimidinylaminoethylamino-2-pyridyl containing compounds, a carboxy alkyl ester, a quinic acid derivative, a caffeic acid derivative, a ferulic acid derivative, or a quinic acid lactone or derivative thereof or pharmaceutically acceptable salt thereof, and combinations thereof in conjunction with the antimicrobial formulations disclosed herein.

In certain embodiments, the therapeutic agent may be one or more of antidiuretics such as thiazide, triamterene, or carbonic anhydrase; vestibular suppressant medications that have been used in controlling vertigo in peripheral vestibular disorders with anticholinergic, antiemetic, and sedative properties: diazepam, meclizine, dimenhydrinate, prochlorperazine, promethazine, and preazepam. In addition to vestibular suppressants several anticholinergic medications may occasionally be useful in managing Meniere's patients, e.g., glycopyrrolate, propantheline, atropine, and scopolamine is useful in ameliorating motion sickness.

In certain embodiments, the therapeutic agent may be a steroid such as dexamethasone or prednisone co-administered with antacids and H2 Blocker to counter their side effects to treat sudden hearing loss.

In certain embodiments, the therapeutic agent may be an agent that modifies post-synaptic glutamate-mediated neurotransmission, either directly by interacting with a glutamate receptor to decrease glutamate binding (e.g., by decreasing glutamate binding to one or more ionotropic receptors or acting as an antagonist to a glutamate receptor such as an NMDA receptor) or indirectly by modulating an endogenous factor which in turn decreases glutamate binding to a glutamate receptor (e.g., by modulating the glycine site of PCP site)m such as NMDA receptor antagonists, 5-HT3R antagonists (e.g., azasetron), AMPA receptor antagonists, and kainate receptor antagonists or components with effects on more than one of said receptor types. Exemplary agents include, but are not necessarily limited to, NMDA-specific glutamate antagonists such as D-2-amino-5-phosphonopentanoate (D-AP5), Dizocilpine (MK 801), 7-chlorokynurenate (7-CK) and Gacyclidine (GK-11). Among the NMDA-antagonists, Gacyclidine is considered one of the preferred compounds. Agents that could be used for the application may also include drugs that mimic or block the action of the lateral efferents, including those that affect neurotransmitters or neuromodulators such as acetylcholine, GAB A, dopamine, enkephalins, dynorphins and calcitonin gene-related peptide. Additionally, all drugs that act on sodium channel activity such as riluzole, dextromethorphan may be used to treat inner ear disorders such as tinnitus in accordance with the present application. The therapeutic agents for treating tinnitus may be based on lidocaine, gabepentin, nortryptline, melatonin, caroverine, baclofen, alprazolam, gacyclidine, 7-chlorokynurenate, or ketamine. Arylcycloalkylamine compounds may be provided in the form of a pharmaceutically acceptable salt. Examples of such a salt include, but are not limited to, those formed with organic acids (e.g., acetic, lactic, citric, malic, fumaric, tartaric, stearic, ascorbic, succinic, benzoic, methanesulfonic, toluenesulfonic, or pamoic acid), inorganic acids (e.g., hydrochloric, nitric, diphosphoric, sulphuric, or phosphoric acid), and polymeric acids (e.g., tannic acid, carboxymethyl cellulose, polylactic, polyglycolic, or co-polymers of polylactic-glycolic acids).

In certain embodiments, the therapeutic agent may be an agent for inducing the self-renewal of stem/progenitor supporting cells, including inducing, promoting, or enhancing the growth, proliferation, or regeneration of inner ear tissue. In certain embodiments, the therapeutic agent may be a pharmaceutically acceptable salt of a GSK3β Inhibitor that comprises a moiety selected from the group consisting of: a maleimide, a pyrrol-2-ones, a pyrazol-3-one, a pyrazoloquinolin-one, a Paullone, a pyridinyl moiety, a pyrimidinyl moiety, triazinyl moiety, imidazolyl moiety, quinolinyl moiety, isoquinolinyl moiety, quinoxalinyl moiety, indazolyl moiety, isoindolyl moiety, pyrazolyl moiety, indolyl moiety, pyrazolinyl moiety, indolinyl moiety, piperidinyl moiety, and morpholinyl moiety; salt of a 1,2,3,4-tetrahydro-[1,4]diazepino[6,7,1-hi]indole; a differentiation inhibitor, e.g., an HDAC inhibitor or a Notch agonist. In some embodiments, the differentiation inhibitor is an HDAC inhibitor or a Notch agonist. In some embodiments, the differentiation inhibitor is valproic acid salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

In certain embodiments, the therapeutic agent can be a salt derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases used in certain embodiments include isopropylamine, diethylamine, ethanolamine, trimethylamine, dicyclohexylamine, choline and caffeine. Salts can have a moiety selected from the group consisting of: a maleimide, a pyrrol-2-ones, a pyrazol-3-one, a pyrazoloquinolin-one, a Paullone, a pyridinyl moiety, a pyrimidinyl moiety, triazinyl moiety, imidazolyl moiety, quinolinyl moiety, isoquinolinyl moiety, quinoxalinyl moiety, indazolyl moiety, isoindolyl moiety, pyrazolyl moiety, indolyl moiety, pyrazolinyl moiety, indolinyl moiety, piperidinyl moiety, and morpholinyl moiety.

In certain embodiments, the therapeutic agent may also comprise an agent to decrease target gene expression encapsulated or incorporated into a nanoparticle. The agent is in an effective amount to decrease the expression of target genes selected from the group consisting of Hes1, Hes5, and MAPK1. The preferred nanoparticle comprises a biocompatible and biodegradable polymer and is more preferably poly(lactic-co-glycolic acid) (PLGA). In one aspect, the agent comprises one or more siRNA molecules sufficient to decrease the mRNA levels of Hes1, Hes5, or MAPK1. The active agent can be short interfering RNA (siRNA) molecules encapsulated in a biodegradable nanoparticle.

In certain embodiments, the therapeutic agent may comprise pharmaceutical compositions for epigenetic modulation of Atoh1 expression to generate sensorineural hair cells (see U.S. Pat. No. 10,603,295 B2), comprising one or more of the following: a Histone Deacetylase (HDAC) inhibitor; a histone methyltransferase (HMT) inhibitor; a DNA methyltransferase (DNMT) inhibitor; a Histone Lysine Demethylase (KDM) inhibitor; an R-spondin; activators of c- and n-myc or Wnt agonists; and/or an inhibitory nucleic acid that specifically reduces expression of Hic1.

In some embodiments, immunomodulating agents, such as an anti-TNF agent, a calcineurin inhibitor, an IKK inhibitor, an interleukin inhibitor, a TNF-α converting enzyme (TACE) inhibitor, or a toll-like receptor inhibitor, may be a part of a therapeutic formulation. In some embodiments, the additional therapeutic agent is a Na/K ATPase modulator, a chemotherapeutic agent, a collagen, a gamma-globulin, an interferon, an anti-microbial agent, an antibiotic, a local acting anesthetic agent, a platelet activator factor antagonist, a nitric oxide synthase inhibitor, an anti-vertigo agent, a vasopressin antagonist, an anti-viral, an anti-emetic agent or combinations thereof.

In certain embodiments, the therapeutic agent may also comprise probiotics L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. rhamnosus, B. adolescentis, B. angulation, B. bifidum, B. breve, B. catenulatum, B. infantis, B. lactis, B. longum, B. pseudocatenulatum, and S. thermophiles.

Suitable media and carriers for the therapeutic agents for topical administration are organic or inorganic substances, which are pharmaceutically acceptable and do not react with the active compounds, for example, saline, alcohols, vegetable oils, benzyl alcohols, alkylene glycols, polyethylene glycols, glycerol triacetate, gelatin, carbohydrates such as lactose or starch, magnesium, stearate, talc and petrolatum, gelfoam, hyaluronic acid, aluminum hydroxide gels (also with muramyl dipeptide), cage-like structures containing cholesterol or saponins, simethicone emulsions, or fibrin glue. The indicated preparations can be sterilized and/or contain ancillary substances such as lubricants, preservatives, such as thiomersal (e.g., at 50%), stabilizers and/or wetting agents, emulsifiers, salts to influence the osmotic pressure, buffer substances, colorants, and/or aromatizing substances. Other examples of carriers include diluents, excipients, suspending agents, adjuvants, vehicles, delivery systems, emulsifiers, disintegrants, absorbants, adsorbents, preservatives, surfactants, colorants, flavorants, emollients, buffers, pH modifiers, thickeners, water softening agents, humectants, fragrances, stabilizers, conditioning agents, chelating agents, sweeteners, propellants, viscosity increasing agents, solubilizers, plasticizers, penetration enhancing agents, film forming agents, antioxidants, stiffening agents, wetting agents, or any mixture of one or more of these components.

Suitable topical excipients and vehicles can be routinely selected for a particular use by those skilled in the art, and especially with reference to one of many standard texts in the art, such as Remington's Pharmaceutical Sciences, Vol. 18, Mack Publishing Co., Easton, Pa. (1990), in particular Chapter 87. For instance, biologically-active agents in accordance with the present application can be combined with enhancing agents which enhance the penetration of an agent.

In certain embodiments, the therapeutic agent may include auris-pharmaceutically acceptable antioxidants, for example, butylated hydroxytoluene (BHT), sodium ascorbate, ascorbic acid, sodium metabisulfite and tocopherol. In certain embodiments, antioxidants enhance chemical stability where required. Antioxidants are also used to counteract the ototoxic effects of certain therapeutic agents, including agents that are used in combination with the otic agents disclosed herein. The ion channel modulator also includes compounds that modulate the expression or post-transcriptional processing of a fluid homeostasis protein, including vasopressin and estrogen-related receptor beta protein. Additionally, vasopressin receptor or estrogen-related receptor beta modulators include compounds that influence vasopressin receptor or estrogen-related receptor beta signaling or downstream functions under the control of the vasopressin receptor or estrogen-related receptor beta, such as aquaporin function. Vasopressin receptor or estrogen-related receptor beta modulating agents includes compounds that increase and/or decrease vasopressin receptor or estrogen-related receptor beta function, including antagonists, inhibitors, agonists, partial agonists and the like.

In certain embodiments, the therapeutic agent may include the active ingredient(s), the biocompatible polymer(s) and, if necessary, adjuvant, carrier, excipient, glidant, sweetening agent, diluent, preservative, dye/colorant, flavor enhancer, surfactant, wetting agent, dispersing agent, suspending agent, stabilizer, isotonic agent, cross-linking agent, solvent, surfactant, or emulsifier, dye (e.g., a Trypan blue dye, Evans blue dye) or other tracer compound, which has been approved by the United States Food and Drug Administration as being acceptable for use in humans or domestic animals adjuvants, may be prepared by any of the methods well known in the art of pharmacy, e.g., by conventional mixing, granulating, confectioning, dissolving or lyophilizing methods. Exemplary pharmaceutically acceptable carriers for therapeutic agents include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical compositions. The polymer compositions of the conduit may be chosen according to the desired release rates of the different active agents and may therefore be same or different in each part. The different parts of the core may be either positioned next to each other or in such a way that one part of the core encases at least partly another part of the core. The different parts of the core may be either spaced from each other and/or may be separated by a separating membrane. The separation membranes may be permeable or impermeable to at least one of the pharmaceutically active agents. In addition, it is possible to use a membrane which is permeable to a first active agent but impermeable to a second active agent.

Drug medium materials can include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

In certain embodiments, other related aspects the one or more therapeutic agents may be selected from the group consisting of drugs, proteins, vitamins, minerals, saccharides, lipids, nucleic acid, peptides, manure, plant nutrients, chemicals, perfumes, fragrances, flavoring agents, animal feed, effervescent gas releasing agents, and combinations and modifications thereof, wherein the drugs are selected from the group consisting of an analgesic agent, an anti-inflammatory agent, an antihistaminic agent, an antiallergic agent, a central nervous system drug, an antipyretic agent, a respiratory agent, a steroid, a local anesthetic, a sympathomimetic agent, an antihypertensive agent, an antipsychotic agent, a calcium antagonist, a muscle relaxant, a vitamin, a cholinergic agonist, an antidepressant, an antispasmodic agent, a mydriatic agent, an anti-diabetic agent, an anorectic agent, an antiulcerative agent, an anti-tumor agent, or combinations modifications thereof, the proteins are selected from the group consisting of an immunoglobulin or fragments thereof, a hormone, an enzyme, a cytokine, a biomolecule, and combinations and modifications thereof.

Dispersing agents, and/or viscosity modulating agents and/or thickening agents are materials that control the diffusion and homogeneity of the otic agent through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose, carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Optional dispersing agents useful in liposomal dispersions and self-emulsifying dispersions of the otic agents disclosed herein are dimyristoyl phosphatidyl choline, natural phosphatidyl choline from eggs, natural phosphatidyl glycerol from eggs, cholesterol and isopropyl myristate.

Solubilizers” refers to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, Transcutol®, propylene glycol, and dimethyl isosorbide and the like.

In certain embodiments, the therapeutic agent may include surfactants, for example, auris-acceptable compounds, such as sodium lauryl sulfate, sodium decussate, Tween 60 or 80, triacetin, vitamin E TPGS, phospholipids, lecithins, phosphatidyl cholines (c8-c18), phosphatidylethanolamines (c8-c18), phosphatidylglycerols (c8-c18), sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

In some embodiments, dyes that are compatible with the drug formulations described herein include Evans blue (e.g., 0.5% of the total weight of an otic formulation), Methylene blue (e.g., 1% of the total weight of an otic formulation), Isosulfan blue (e.g., 1% of the total weight of an otic formulation), Trypan blue (e.g., 0.15% of the total weight of an otic formulation), and/or indocyanine green (e.g., 25 mg/vial). Other common dyes, e.g., FD&C red 40, FD&C red 3, FD&C yellow 5, FD&C yellow 6, FD&C blue 1, FD&C blue2, FD&C green 3, fluorescence dyes (e.g., Fluorescein isothiocyanate, rhodamine, Alexa Fluors, DyLight Fluors) and/or dyes that are visualizable in conjunction with non-invasive imaging techniques such as MRI, CAT scans, PET scans or the like (e.g., Gadolinium-based MRI dyes, iodine-base dyes, barium-based dyes or the like) are also contemplated for use with any otic formulation described herein. Other dyes that are compatible with any formulation described herein are listed in the Sigma-Aldrich catalog under dyes (which is included herein by reference for such disclosure). In some embodiments, concentration of a dye in any otic formulation described herein is less than 2%, less than 1.5%, less than 1%, less than 0.5%, less than 0.25%, less than 0.1%, or less than 100 ppm of the total weight and/or volume of any formulation described herein.

In certain embodiments, the therapeutic agent may be embedded in carbon-based materials such as carbon nanotubes and fullerenes and their suspensions; metal organic and covalent organic framework (MOF and COF, respectively) particles; zeolites; nanoclays and layered silicates (e.g., montrillomonite, kaolinite, laponite, halloysite, bentonite, hectorite, laponite, sepiolite, saponite, and vermiculite); polymers compounds such as Poly(lactic acid), Poly(glycolic acid), Poly(lactic-co-glycolic acid), or dendrimers, hydrogels, biomacromolecular scaffolds, solid lipid nanoparticles or liposomes, microemulsions or nanoemulsions. In certain embodiments, the therapeutic agent may contain molecules that either release or target biological entities including monoclonal antibodies, nanobodies, aptamers, proteins or enzymes or peptides.

In certain embodiments, the therapeutic agent may be embedded in creams, gels, liniments, balms, lotions, ointments, and patches.

Pharmaceutical formulations may have stimuli-responsive components, including but not limited to thermal, pH-responsive, redox-responsive, light-responsive, magnetic field-responsive, electroresponsive, or enzyme-responsive components. Therapeutic agents may be activated by mechanisms including but not limited to disassembly mechanisms, linker cleavage mechanisms, swelling mechanisms, cap removal or gas generation mechanisms. In one embodiment, the nanoparticle further comprises SPION coated with oleic acid in order to render the nanoparticle susceptible to movement or transport by applied magnetic gradients to a desired location of the inner ear. The magnetic nanoparticles can include a magnetic core comprising one or more metals such as ferrite (e.g., Fe₃O₄, y-Fe₂O₃, and CoFe₂O₄). Optionally, the nanoparticles can further include a functionalized coating fashioned from a polymer, hydrogel, polyethylene glycol, glucuronic acid, glycine, or matrix-like materials. The functional coating can serve as a substrate for any number of additional components, including detectable markers (e.g., fluorescent tags such as fluorescein isothiocyanate (FITC) or Rho), targeting agents, and drugs or therapeutic agents.

The therapeutic agent can be an anti-inflammatory or anti-microbial agent (e.g., an antibiotic, anti-fungal, or anti-parasitic drug). In some embodiments, the therapeutic agent can be nucleic acid constructs that express CYLD or a biologically active variant thereof (e.g., a variant including the catalytic domain), nucleic acids that inhibit the expression of a negative regulator of CYLD (e.g., PDE4B or JNK2), nucleic acids that modulate the expression of downstream CYLD targets (e.g., Akt, by inhibiting or promoting the expression of the downstream target). Nanoparticles can be conjugated with a material selected from the group consisting of lipids, proteins, growth factors, growth hormones, antioxidants, free radical scavengers, steroid preparations, and metabolically active substances; an otologic therapeutic device includes the same categories of substances and chemotherapeutic drugs.

### B. Priming Agent

As described herein, the priming agent refers to a material that modifies one or more properties of the conduit including the physical properties such as viscosity, wettability, heterogeneity, size, shape, porosity, texture, permeability, or combinations thereof; optical properties that includes light absorption; chemical properties that includes pH, reactivity, or combinations thereof; mechanical properties that includes elasticity; thermal properties that includes glass transition temperature, thermal conductivity, or combinations thereof.

The priming agent may also be activated where the term “activation” may indicate application, addition, release, secretion, changing activity, availability, local concentration, with time or in response to a stimulus.

In certain embodiments, the priming agent is selected from the group consisting of carrier liquid for the therapeutic agent, surfactants, low-surface-energy liquids, lubricating agents, acids and bases, emulsifiers, rheology control agents, flow-promoting agents, extenders, defoaming agents, plasticizers, thickeners, heat stabilizers, porogens, levelling agents, anti-cratering agents, fillers, UV absorbers, curing agents, diluents, adjuvants, buffering agents, moistening agents, anti-oxidants, oxidizing or reducing agents, mono-dentate or multi-dentate or chelating ligands, crosslinking agents, solubilizing agents, organic and inorganic solvents, SPION, cubosomes, polymersomes, preservatives, polyglycols, long chain hydroxy polyanionic polysaccharides, long chain nitrogen containing polymers, long chain hydroxy polyanionic polysaccharides, glycerol esters, glycerol ethers, glycols, glycolesters and glycolethers, glycerol, monoacetin, diacetin and diacetone alcohol, poly(lactams), polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, waxes, organic co-solvents, alkylene glycols, ethoxylated or propoxylated alcohols, amines, waterless colloid silica, povidone, talc, monolein, magnesium stearate, flavouring agents, lactose, glucose, sucrose, starches, cellulose, derivatives of cellulose, tragacanth, malt, talc, partially or fully fluorinated liquids, oils, cocoa butter, animal fat, vegetable fats, paraffins, silicones, bentonites, silicic acid, zinc oxide, glycols, polyols, esters, agar, buffering agents, alginic acid, pyrogen-free water, isotonic saline, Ringer's solution, ethyl alcohol, phosphate buffer solutions, acacia, colloidal silicon dioxide, calcium glycerophosphate, calcium lactate, maltodextrin, glycerine, magnesium silicate, cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, propylene glycol, dimethyl isosorbide, polyoxyethylene fatty acid glycerides, vegetable oils, polyoxyethylene alkylethers, alkylphenyl ethers, and combinations thereof.

In certain embodiments, the priming agents are selected from the group consisting of accelerators, acid scavengers, activators, adhesion promoters, antiblocking agents, antifogging agents, antimicrobials, catalysts, coagulants, colorants, curing agents, thermal stabilizers, viscosity control agents, oxidizing or reducing agents, mono-dentate or multi-dentate or chelating ligands, solubilizing agents, organic and inorganic solvents, acids and bases, pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, azobenzenes, diarylethenes, fulgides, overcrowded alkenes, plasmonic, photothermal or magnetic nanoparticles, catalysts, enzymes, agents capable of extracting therapeutic components from the conduit, and combinations thereof.

### C. Activating Agent

As described herein, the activating agent is a material capable of releasing the therapeutic agent toward a portion of the inner surface of the conduit upon activation of the activating agent.

The activating agent may also be activated, where the term “activation” may indicate application, addition, release, secretion, changing activity, availability, local concentration, with time or in response to a stimulus.

In certain embodiments, the activating agents are selected from the group consisting of accelerators, acid scavengers, activators, adhesion promoters, antiblocking agents, antifogging agents, antimicrobials, catalysts, coagulants, colorants, curing agents, thermal stabilizers, viscosity control agents, oxidizing or reducing agents, mono-dentate or multi-dentate or chelating ligands, solubilizing agents, organic and inorganic solvents, acids and bases, pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, azobenzenes, diarylethenes, fulgides, overcrowded alkenes, plasmonic, photothermal or magnetic nanoparticles, catalysts, enzymes, agents capable of extracting therapeutic components from the conduit, and combinations thereof.

### D. Reversal Agent

As described herein, the reversal agent is a material that reverses or changes one or more properties of the conduit after the therapeutic agent to prepare the conduit for the next phase of the therapeutic agent delivery.

The reversal agent may also be activated, where the term “activation” may indicate application, addition, release, secretion, changing activity, availability, local concentration, with time or in response to a stimulus.

In some embodiments, the reversal agent is selected from the group consisting of stoichiometric reagents, chelators, Ringer's solution, saline solution, water, oils, liquid crystal molecules, thermoresponsive molecules, supramolecules, biomolecules, antibodies, antigens, fluorophores, sterols, surfactants, and combinations thereof.

In some embodiments, the reversal agent is selected from the group consisting of pigments, dyes, surfactants, emulsifiers, sedimentation inhibitors, antioxidants, organic co-solvents, coalescents, allosteric modulator, stoichiometric reagents, cross-linking agent, aliphatic stabilizing compounds, alicyclic stabilizing compounds, aliphatic compounds, aldehydes, UV absorbers, photoswitchable molecules, photochromic compounds, spiropyrans, diarylethenes, fulgides, overcrowded alkenes, plasmonic, photothermal or magnetic nanoparticles, catalysts, enzymes and combinations thereof.

In some embodiments, the reversal agent is able to cause an endothermic or exothermic reaction within or on the surface of the conduit, and such reaction causes one of a transitory modification or a permanent modification of a surface tension between the drug formulation and the conduit, or a change in other physical properties. Some non-limiting examples for exothermic reactions would include: Calcium Oxide+H₂O, Calcium Chloride+H₂O, Iron+Air (O₂), Sodium Acetate, Magnesium Iron Alloy+H₂O. Some non-limiting examples of endothermic ingredients that upon combining with water, generate endothermic reaction are: inorganic salt or inorganic hydrates of ammonia, alkali metals, calcium, urea, simple saccharides and mixtures thereof, suitable inorganic salt are crystalline phosphates, sulfates, carbonates, nitrates, and the like, e.g., sodium phosphate salts, sodium ammonium phosphate salts, and ammonium phosphate salts, disodium hydrogen phosphate, sodium dihydrogen phosphate, trisodium phosphate, and hydrates thereof, sodium ammonium hydrogen phosphate, diammonium hydrogen phosphate, ammonium dihydrogen phosphate, triammonium phosphate, and hydrates thereof, sodium carbonate, sodium hydrogen carbonate, potassium sodium carbonate, sodium chromium carbonate, sodium scandium carbonate, sodium cerium carbonate, sodium sulfate, and hydrates of the above. In one embodiment, solid/solid chemical reactions can occur which are endothermic. Examples of ingredients used for such reaction would involve hydrated inorganic salts in their solid form reacting with selected solid ammonium salts, e.g. barium hydroxide octahydrate with ammonium chloride.

### IV. Case Studies of Different Conduit-Drug/Therapeutic/Plug Formulation Interactions

### A. Case 1: Modification of Conduit's Geometry/Material Composition for a Given Drug Formulation

In certain embodiments, the patient is prescribed to take a single drug formulation, e.g., antibiotic Ciprodex®, which is then introduced via topical administration through the lumen. For a given drug formulation, one can modulate the conduit's geometry and material composition (as well as morphology, wettability, gating via stimuli).

In some embodiments, the parameter space for design of conduits with desired fluid flow characteristics is provided by the Young-Laplace equation, which dictates that the maximum pressure required for a fluid to penetrate either end of the conduit:

$$Δ P = - \frac{2 {γ}_{eff}}{{R}_{t} ( h )} cos ( {θ}_{adv} ( h ) + {θ}_{f} ( h ) ) ,$$

where ΔP is the pressure difference across the fluid interface and the effective surface tension of the fluid Y_{eff}. One could modify: a) the effective surface tension of the phase in contact with air Y_{eff}, b) the radius of the conduit Rₜ(h), c) the advancing angle of the three-phase front θ_{adv}(h), and d) lumen flare angle θ_{f}(h). Thus, to facilitate transport of specific liquids with surface tension fluid Y_{eff} (e.g., ˜40 mN/m for antibiotic solutions), geometric parameters can be modularly tuned to reduce the pressure difference across the fluid interface, thus significantly enhancing fluid transport. Introducing gradients in curvature and radius along the length of the conduit further allows for selective transport in each direction. Based on these design principles, numerical simulations were conducted to identify the geometry, for a given conduit radius, for simultaneously (1) increasing the therapeutic agent elution through the conduit (2) maintaining water impermeability and (3) promoting mucin drainage in the opposite direction.

In some embodiments, the conduits can be optimized for a selective transport of a certain fluid with desired physical or chemical properties, while optimizing for multiple other criteria including exclusion of other non-desirable fluid (e.g., environmental water) to prevent carrying any infectious debris into the middle ear from the outer ear canal.

In certain embodiments, the geometries of the curved conduits can be optimized, without changing the conduit diameter, for an efficient transport of therapeutic agent by varying surface tension. The plurality of these conduits has radius at sub-capillary scales (less than 3 mm), and conduit sub-centimeter lengths to ensure pressure-gradient-driven flow that is governed by surface tension (and viscosity) of the therapeutic fluid. Alternatively, these conduits can be optimized for a selective transport of a certain drug formulation with certain physicochemical properties and not the other drug formulation with other physicochemical properties. In some embodiments, the conduits can have bidirectional properties to serve both the purpose of drug delivery into and drainage of any other biofluid from the middle ear or inner ear. There are many instances where the patient requires the transport of biofluids such as blood, pus, effusion, etc. to be drained from the middle ear space. The unidirectional design can be modified into a bi-directional design that not only facilitates the transport of drugs into the middle ear space but also removal of undesirable fluids from the middle ear space.

In some embodiments, the mechanisms to control drag on surfaces are utilized to manipulate the fluid transport. For example, the conduits can utilize surface modifications such as the use of a stable lubricious layer of Newtonian or non-Newtonian fluid infused or non-infused within the matrix of the material, or other surface modifications (e.g., treatments such as hydrophobic, hydrophilic, amphiphilic functional groups, charged surfactants, lipid-phase additives, and other components, including gradient compositions), to allow for the optimization of the Young-Laplace pressure of the conduit, and thus optimize the conduit for a higher-flow-rate/lower-pressure-barrier regime, and allow for coupling of the specific therapeutic agent and the corresponding fluid.

Some embodiments involve a use of non-Newtonian or viscoelastic infusing fluids that add benefits such as a greater drag reduction in shear thinning regimes. An example is how mucus is infused between villi structures in intestinal surfaces that aid in transport of solids and liquids. Not only do the non-Newtonian infusing liquids significantly reduce frictional drag forces in transport, but they can be tuned to add shear thinning or thickening versatility to the surface. This can be leveraged with textured and “moving walls” to facilitate control over the flow. As a representative example, viscoelastic mucus can be modeled using the Oldroyd-B model as with assumption of no shear thinning. The dimensionless Weissenberg number (Wi) dictates the elastic to viscoelastic behavior. The simulation carried out in COMSOL (using Reynolds number (Re) set to 0.001 and the Wi number was computed via

$$Wi = λ \frac{U}{R} ,$$

where λ is the relaxation time, U is an average inlet velocity of the fluid, and R is the radius of the conduit has demonstrated that the drag was minimized for Wi of ˜0.7. Furthermore, drag reduction and wall strain are heavily dependent on the pattern or texture of the wall. In cases for gut villi, there is a conclusive benefit of having a viscoelastic fluid lining. For a non-patterned surface, this frictional drag difference can be minimal and may only offer some drag reduction. The greatest benefit of this approach is realized when a conduit has a partial occlusion in which the viscoelastic infusing fluid can pose significant drag reduction benefits. For example, ridge structures and inverted prisms textures showed up to 14% drag reduction for viscoelastic fluid infusion compared to Newtonian fluids. These benefits can also be coupled with embodiments of conduit which impose movable flaps to promote or restrict flow.

In some embodiments, the textures can be shaped as various macro- and microstructures with various shapes, including but not limited to posts, rods, plates, stars, whiskers, antennas, and others.

In some embodiments, the valve is selected from one of a stimuli-responsive polymer, a gas-selective mobile membrane, stimuli-responsive cilia-like and hair-like fibers, platelets, pillars, reconfigurable tunable nano- or microstructures with functionalized tips or functionalized pillars, and combinations thereof.

In some embodiments, the conduit includes one or more of a hydrogel, a chemically crosslinked polymer, a supramolecular polymer, a metal, a metal oxide, a porous material, geometrically-patterned pores or channels in a material, membranes and sponges, colloid- and surfactant-templated pores, grooves and ridges, periodic and aperiodic arrays of indentations, nano- and microstructures: nanoforest, nanoscale patterned films, microplatelets, micropillars, and microridges.

In certain embodiments, the use of cilia like structures can be used as pinning points within the lumen.

In certain embodiments, cilia-like structures can be used in combination with radial change through the lumen to prevent the fluid from exiting either end.

In certain embodiments, the texture change includes increased roughness, decreased roughness, formation of grooves, formation of raised structures, formation of depressed structures, texture due to texture agent additives, e.g., micron-sized particles (in the range between 1 and 1000 μm).

In other embodiments, the tips of microstructures are also modified with conductive materials. In certain embodiments using electrical conduction, the electric conduction of the surface or the whole system can be controlled by chemically-induced mechanical actuation of the microstructures.

In certain embodiments, the self-modulated, adaptively reconfigurable, tunable nano- or microstructures have appropriately functionalized (chemically or physically) tips that are embedded in a hydrogel. This dynamic system incorporates the movement of “skeletal” high-aspect-ratio microstructures (posts, blades, etc.) by a polymeric “muscle” provided by the swelling/contracting capabilities of the hydrogel in which the microstructures are embedded. In certain embodiments, the layers are arranged vertically, one stacked over the other. In certain embodiments, the system can also be designed horizontally with these two layers positioned side-to-side.

In some embodiments, a conduit is coated or infused with shear-thinning non-Newtonian fluid. As a result, conduit has reduced drag frictional forces for higher shear rates compared to Newtonian fluid infusions. Hence the net pressure required for flow and the average velocity of the flow increases. This reduces the time the patient is required to stay in an administration specified position.

Another embodiment can be the use of two viscoelastic fluids to infuse different hemicylinders (or halves of the conduits) of the conduit. For example, having two different viscoelastic behavioral infused fluids would allow for the therapeutic agent to mix while transporting. This is enabled by one hemicylinder of the conduit responding differently (causing asymmetric drag) on the fluid being transported compared to the other half. Hence, the curl of the fluid will increase allowing mixing. This can be used in applications when two formulations are administered simultaneously where the mixing is required as the fluids transport through the lumen. An example of such can be a gelling formulation in which the gelation begins when two parts are mixed, however, to promote the fluid flow these parts are administered separately but simultaneously. In such a case, mixing of the fluids as they transport through the lumen is desired. In this embodiment, the compositions are used that comprise a glycosaminoglycan (e.g., a hyaluronan, hyaluronic acid, hyaluronate, sodium hyaluronate, dermatan sulfate, karatan sulfate, chondroitin 6-sulfate, heparin, etc.) in combination with at least one component selected from i) polyglycols (e.g., polyethylene glycol), ii) long chain hydroxy polyanionic polysaccharides (e.g., dextran, sodium alginate, alginic acid, propylene glycol alginate, carboxymethyl cellulose and carboxyethyl cellulose, hydroxyl ethyl starch, hydroxyl propyl methyl cellulose, hydroxy propyl ethyl cellulose, hydroxy propyl cellulose, methyl cellulose, polylysine, polyhistidine, polyhydroxy proline, poly ornithine, polyvinyl pyrolidone, polyvinyl alcohol, chitosan, etc.) and iii) long chain nitrogen containing polymers (e.g., Polylysine, Polyvinylpyrrolidone, and polyvinyl alcohol). Hyaluronic acid can also be stabilized with long chain hydroxy polyanionic polysaccharides (e.g., sodium alginate, alginic acid, propylene glycol alginate, carboxymethyl cellulose and carboxyethyl cellulose, etc.) in an aqueous solution of pH 5.0-9.0. The viscosity of the hyaluronic acid in aqueous solution at pH 5.0-9.0 can be increased by an order of magnitude or more with the addition of one or more long chain hydroxy polyanionic polysaccharides and such hyaluronic Acid including long chain hydroxy polyanionic polysaccharides possesses a non-Newtonian fluid characteristics (shear thinning effects).

In some embodiments, a lubricious hydrophilic coating, wherein the coating is durable and highly lubricious when in contact with body fluids, is introduced. In one embodiment, the coating comprises a polymerized base coat and a hydrophilic top coat, where the base coat has a binding component which binds to the hydrophilic compound of the top coat, and a grafting component which binds to the binding component and to the device. In another embodiment, the coating comprises a blend of a hydrophilic compound, a grafting component, and salt, wherein the polymerized grafting component contains uncrosslinked domains.

The grafting component is selected from the group consisting of vinyl compounds, acrylate compounds, and allyl compounds, such as any oligomer or monomer with one or more vinyl, acrylate or allyl double bonds. Exemplary of the vinyl compounds are di-vinyl benzene, n-vinyl pyrrolidone, and triethylene glycol divinyl ether. Exemplary of the acrylate compounds are tri-methylol propane tri-acrylate, pentaerythritol tetra-acrylate, and Bisphenol A. ethoxylate diacrylate. Exemplary of the allyl compounds are allyl ether, di-allyl maleate, and tri-allyl isocyanurate. The binding component and hydrophilic compound have functional groups capable of binding to one another, so that the hydrophilic compound will be securely bound to the medical device through covalent attachment to the binding component. In one embodiment, the binding component is selected from the group consisting of polyaziridine and polycarbodiimide resin compounds, and the top coat is a hydrophilic polymer having carboxyl groups capable of binding to the binding component. Exemplary of the polyaziridine compounds are tri-aziridine oligomer, such as Zeneca cs-100 available from Zeneca Resins. Exemplary of the carbodiimide compounds are XL-29SE available from Union Carbide. The hydrophilic compound is a polymer showing appreciable water absorption and containing carboxyl groups, including but not limited to, polyacrylic acid, alginic acid, carboxy methyl cellulose, and hyaluronic acid.

In another embodiment, the binding component comprises an aldehyde compound and the top coat is a hydrophilic compound having amine groups. Exemplary of such aldehyde compounds are glutaraldehyde, cinnamaldehyde, and acrolein. Exemplary of the hydrophilic compound are a (co)monomer selected from the group consisting of 2-aminoethyl acrylate, 2-aminoethyl methacrylate, and N-(3-aminopropyl)methacrylamide; or a polymer of at least one of said (co)monomers co-polymerized with hydrophilic monomers selected from the group consisting of acrylamide, di-methyl acrylamide, and N-vinyl pyrrolidone; or a peptide having a secondary basic group for reaction with the aldehyde of the binding component, such as arginine, glutamine, and histidine, which include but are not limited to gelatin, hirudin, and albumin; or polyethylenimine.

In another embodiment, the binding component is an isocyanate compound and the top coat is a compound showing appreciable water absorption and containing hydroxy or amine groups. Exemplary of such isocyanate compounds are an aliphatic or aromatic isocyanate monomer, biuret or isocyanurate oligomer, or polyol or polyamine chain extended variant of such starting materials as 1,6 hexamethylene diisocyanate, isophorone diisocyanate, toluene diisocyanate, diphenylmethane-diisocyanate, bis(4-isocyanato cyclohexyl) methane. The isocyanate compound can also be the monomer or polymer made from allyl isocyanate or other such monomers. Exemplary of the hydrophilic compound are poly(vinyl alcohol), hydroxy propyl cellulose, hyaluronic acid, a peptide having a secondary basic group for reaction with the isocyanate of the binding component, and a copolymer blend of a first monomer selected from the group consisting of vinyl and acrylic monomers and a second monomer selected from the group consisting of hydroxy and amine monomers. Examples of the peptide include but are not limited to gelatin, hirudin, and albumin, and examples of the copolymer blend hydrophilic polymers include but are not limited to an 80/20 mixture of acrylamide and hydroxy ethyl methacrylate.

In some embodiments, a conduit's macroscopic geometry in conjunction with micropatterning. In instances, these micropatterning, such as flaps, can constrict the lumen's effective diameter and prevent fluid transport. However, upon breaking Young-Laplace capillary pressure, the fluid flow exerts normal stress onto the flaps to widen the lumen diameter further promoting fluid flow (a). In instances (b), this embodiment can have the flaps angled in the direction desirable for fluid flow and therefore prevent backflow or reverse flow of any fluid; a valueless one directional conduit. In other instances, this embodiment can serve as a selective transport conduit based on stress applied to microstructures, where fluids imposing higher normal and shear stress open the lumen. This opening of the lumen furthers the flow of the fluid, the flap position serves as a filter which bends depending on the stress.

Another embodiment can be the use of a conduit with flaps infused with shear thinning non-Newtonian fluid. With higher pressures, the normal and longitudinal stress vectors are minimized allowing the flaps to have reduced drag on the fluid in contact (i.e. the therapeutic agent), once the pressure barrier is overcome (i.e. the fluid is in motion); however, this embodiment does not prevent undesirable flow of the fluid (i.e. the therapeutic agent) as the shear thinning effects are realized only when the fluid has overcome the minimal pressure barrier dictated by the constricted lumen (Young-Laplace pressure).

In one embodiment, a transport device is in fibral form. The device is composed of tiny fibers that can extend to the round window to transport drug formulation via a fluidic channel. The embodiment can absorb and swell upon delivery of a therapeutic agent to be transported; the fluid is then transported through the fibers due to anisotropy in convective flow and capillary flow, wicking behavior (dimensional confinement). Upon reaching the end of the fiber, the drug formulation can diffuse out the fiber allowing efficient and unhindered transport to parts within the ear inaccessible by traditional means of drug transport. In this embodiment, minimal therapeutic agent is lost to drainage via the eustachian conduit and side reactions or contamination is mitigated from pristine transport.

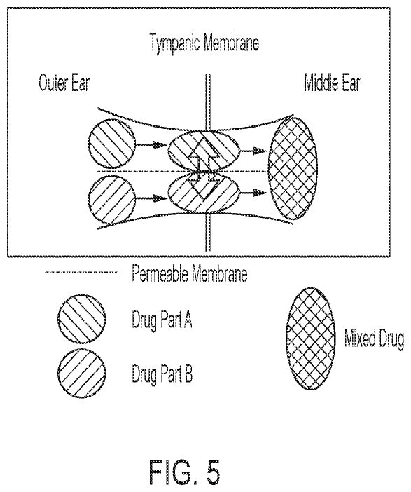

In one embodiment, a conduit has a thin permeable or semi-permeable membrane that allows two drug components to be administered on separate hemicylinders (or parts) of the conduit (FIG. 5). Upon transporting through the lumen, the diffusion of the drugs through the permeable membrane allows the drug components to mix and become an active therapeutic agent in the middle space. This embodiment can be further tuned to have an impermeable membrane to certain moieties allowing selective mixing and/or no mixing. This enables formulations to be made with specific transport properties in mind (such as low viscosity) before mixing but becoming active and gel-like upon mixing in the middle ear space.

In one embodiment, the conduit's cross section represents a periodic or aperiodic arrangement of compartments.

### B. Case 2: Modification of the Drug Formulation for a Given Conduit Geometry/Material Composition

In this non-limiting possible scenario, the patient is prescribed to take a single drug formulation, e.g., antibiotic Ciprodex®, which is then introduced via topical administration through lumen. For a given conduit geometry/material composition, the formulation for the topical drug formulation or the therapeutic agent itself can be modified to allow it to be introduced at controlled rates via drug formulation modification techniques and surface priming. This scenario can be useful if, for example, the doctor has a preferred conduit shape.

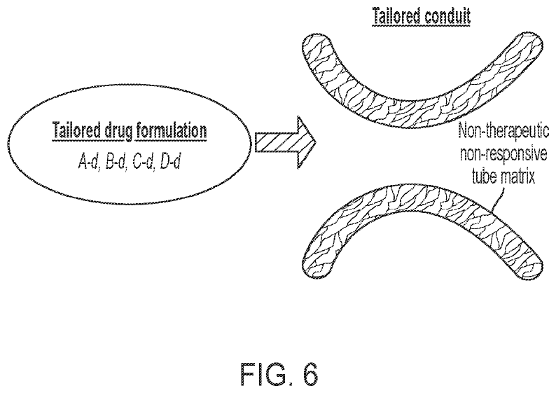

In the way that a conduit can be modified to match the surface tension of the fluid for ideal transport, a single conduit can be used for transport of multitude of therapeutic agents by the manipulation of the drug formulation fluid properties such as viscosity, surface tension, density, hydro-lipophilic balance, tonicity, and more. In one example in FIG. 6, the drug formulations contain one or more of the following compounds: A-d is a therapeutic compound drug (“therapeutic agent”) that needs to be delivered for treatment of a variety of disorders, e.g., ear disorders, B-d is any compound (“priming agent”) that modifies conduit wettability or size, or shape or porosity in preparation for drug transport, C-d is a compound (“activating agent”) that has the ability to “activate” drug delivery through the conduit tube/drug on demand (either just prior to or during drug administration) through adjusting drug/conduit surface interactions (for example, photothermal agents such as gold nanoparticles embedded in the conduit or incorporated in the topically administered drug that can produce heat locally to reduce surface tension). D-d (“reversal agent”) reverses or changes conduit shape, size, porosity or wettability after drug transport to prepare it for the next phase of delivery.

In certain embodiments, the transport of the drug formulations can be facilitated by optimization of the fluid properties to match efficient flow criteria for a conduit material geometry. For instance, a conduit that is optimal for transporting low viscosity and/or low surface tension fluid can also be used for transporting other fluids given they are modified by a medium phase, such as viscosity and surface tension modifiers. These changes to the composition can be embodied in both the inactive phase of the transport i.e. the medium, or the active phase i.e. the therapeutic agent.

In certain embodiments, the liquid can be modified to facilitate efficient transport in a given geometry. For instance, a drug formulation can be modified by introducing medium changes to match the ideal surface tension of transport for a given conduit.

In some embodiments the drug modifier can cause a temporary or long-term expansion of the conduit material for facilitating the flow.

In some embodiments, the conduit allows for gelation drug formulations to transport efficiently and only start reacting once nearly transferred through the conduit lumen. The therapeutic agents embedded into polymers like poloxamer (e.g., Otiprio) may undergo gelation during or after the administration, leading to occlusion of devices. To mitigate these risk factors, a conduit embodiment as described above can serve to transport the components of the drug while ensuring the reaction begins once it has nearly cleared the conduit lumen. This design poses benefits such as reduced tube failures, improved drug efficacy, and stable drug formulations.

### C. Case 3: Optimization of Conduit's Geometry/Material Composition for a Given Drug Formulation and Multiple Other Constraints

In some embodiments, due to the vast dimensionality of the engineering designs possible for the conduit-drug pairing, a method to evaluate the best conduit, drug formulation properties, and additional chemical or physical agents (e.g., primer, stimuli, etc.) can be employed using the machine learning inspired function, i.e., the logistic function.

An example of a unidirectional conduit and important parameters when designing the conduit-drug combination is presented for a hypothetical patient needing antibiotic drops for treatment of otitis media with effusion. In this example, it is desirable to have minimal pressure barrier against therapeutics to transport the therapeutics, but maximum pressure barrier against water to ensure water is not transported. Additional factors are considered such as flow rate of drug transport and mass of conduit (as it impacts the tympanic membrane's function directly). The logistic function has been modified to accommodate for these multitude of criteria:

$$TS = {\prod }_{i}^{n} {f ( {x}_{i} )}^{\frac{{q}_{i}}{{\sum }_{j}^{m} {q}_{j}}} = {\prod }_{i}^{n} {( \frac{{l}_{i}}{1 + {e}^{{k}_{i} ( {x}_{i} - {x}_{i , 0} )}} )}^{\frac{{q}_{i}}{{\sum }_{j}^{m} {q}_{j}}}$$

The difference between xᵢ and x_{i,0} indicates the performance difference between the current conduit design and a chosen standard conduit for a single design parameter. The difference is multiplied by the bias factor kᵢ that indicates the sensitivity of the parameter. The numerator lᵢ determines the max score achievable for the parameter; this is typically set to 1. The exponent numerator (qᵢ) determines the importance of the design parameter compared to the other parameters Σⱼ^{m}qⱼ. When choosing a conduit design, the conduit is optimized in geometry, material, and surface coating for a particular application in a method to optimize the score. Higher score conduits indicate the design is better suited to accomplish the desired functions. Conduits with scores nearly 1 are ideal whereas conduits with scores near 0.5 are similar to standard conduits. Conduits with scores of nearly 0 are not suited for the application as they are worse than standard conduits. This method of evaluation allows engineering design to be guided by physical parameters and application specific criteria.
D. Case 4: Modification of Conduit with a Set of Therapeutic Formulation Embedded into the Conduit's Matrix or Lumen to Induce Sustained Release

In certain embodiments, the conduits can have one or more types of therapeutic agent molecules embedded via suspension into the conduit material, deposited non-covalently of the conduit surface, or incorporation into organic or inorganic vehicles such as liposomes or nanotubes that are suspended in the polymer matrix or oil component of the conduit material. Sustained release of the therapeutic agent is achieved when there is a gradient in the therapeutic agent concentration across the conduit and the middle ear space. Here, the sustained release denotes a drug preparation in a fluid or solid drug carrier or composite, e.g., fluid, microparticle, nanoparticle, vesicle, capsule, and other physical compartments with different coatings, within the volume or surface of the conduit material and containing portions or the therapeutic agent that release their contents steadily or on-demand over a continuous period.

The difference between xᵢ and x_{i,0} indicates the performance difference between the current conduit design and a chosen standard conduit for a single design parameter. In some embodiments, one or more therapeutic formulations (including in the form of solid nanomaterials or immiscible liquids pockets) embedded within the conduit matrix awaiting release can be spatially patterned or infiltrated with gradients within the conduit material, both radially and along the length of the conduit utilizing various therapeutic agent carriers/vehicles carrying active chemical or biological therapeutic components, prodrugs and codrugs (including but not limited to antibiotics, steroids, genes, proteins, monoclonal antibodies, nanobodies, aptamers, proteins, peptides) and release mechanisms, including but not limited to disassembly, swelling, linker cleavage, cap removal, and gas penetration.

The difference between xᵢ and x_{i,0} indicates the performance difference between the current conduit design and a chosen standard conduit for a single design parameter. For example, antibiotic compounds or other compounds such as corticosteroids can be embedded on the curved end facing the outer ear and/or middle ear, and anti-inflammatory compounds can be embedded at the middle junction where it is attached with the tympanic membrane to, for example, promote healing. The therapeutic agent encapsulated at different regions can be similar or different depending on the condition to be resolved. The porosity, matrix composition, any infused liquid components can be patterned to induce spatial gradients in loading of therapeutic formulations. Thus, conduits with asymmetric therapeutic agent release profiles on either end of the conduit e.g., facing inward vs outward may be created.

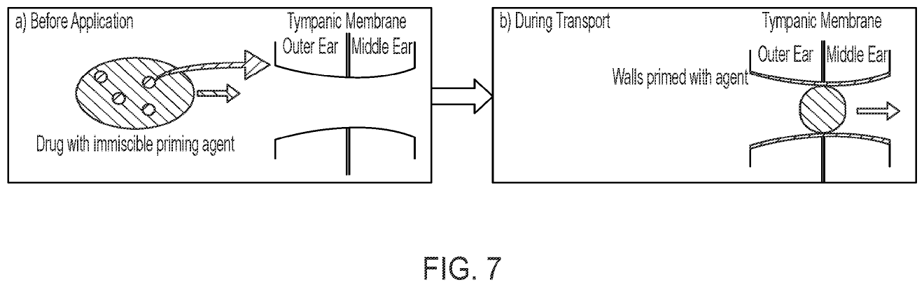

The difference between xᵢ and x_{i,0} indicates the performance difference between the current conduit design and a chosen standard conduit for a single design parameter. In some embodiments the medium/carrier for the therapeutic agent can include an immiscible priming agent that modifies the surface of the conduit, such as oil, which can be used to serve two purposes: a solvent and carrier for the therapeutic agent, and an agent to modify (e.g., lubricate) the surface of the conduit as shown in the FIG. 7.

See Case 6 for embodiments in which the release and transport of the therapeutic formulation is further affected by introduction of one or more administrations of the drug formulation.

(a) Case 4.1. Changes in Conduit Shape and/or Wettability During Passive Release of Therapeutic Agent to Control Release Profile

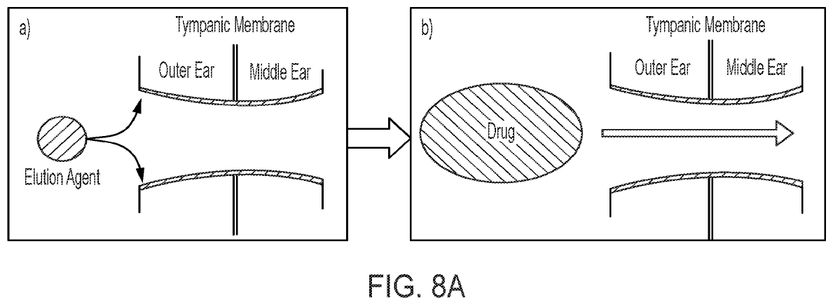

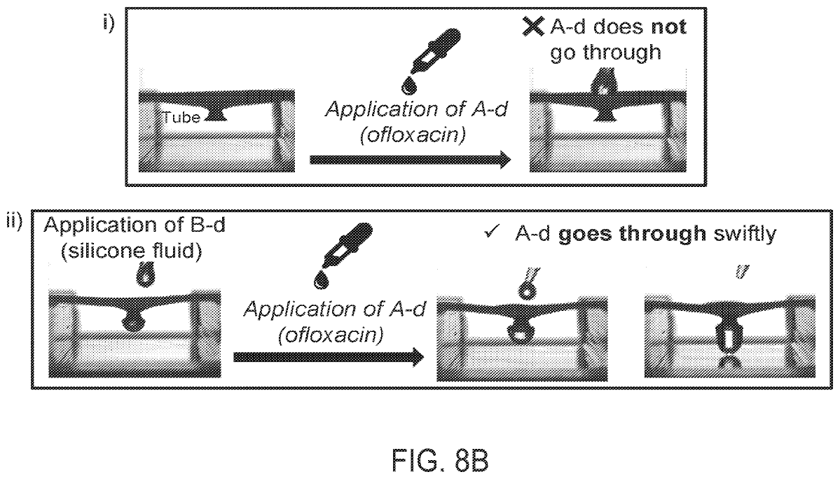

In some embodiments, the conduit matrix can be embedded with one or multiple fluid (e.g., oil) or vapor components that, upon release, create gradients in surface tension, pH, heat (via exothermic reactions) that further guide the diffusion, elution and spreading of remaining drug formulation or therapeutic agent of the drug formulation along the length of the conduit (FIG. 8A). These gradients can be introduced through inhomogeneous therapeutic agent gradients or vasculature present within the matrix of the conduit. FIG. 8B demonstrates experimental validation of the mechanism of eluting the A-d, a therapeutic compound drug (e.g., ofloxacin), by introducing first the B-d priming agent (e.g., silicone fluid) that modifies conduit wettability to facilitate drug transport. Before applying B-d the tube did not show any transport (FIG. 8A panel (i)), and after applying B-d, the tube immediately allowed for transporting the A-d (FIG. 8A panel (ii)).

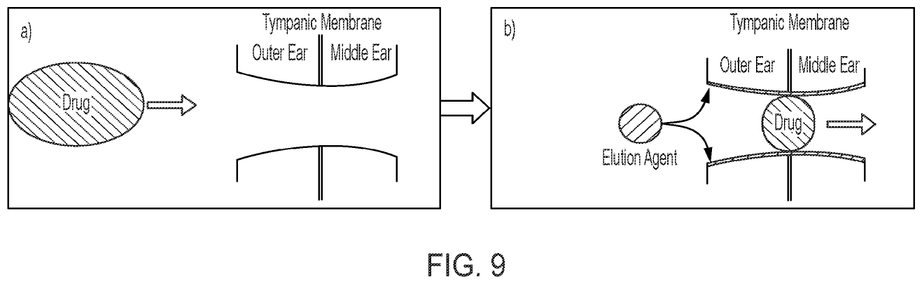

In some embodiments, the priming agent B-d or a “stimulus” for C-d can be introduced after A-d is introduced. It may be used to solubilize and propel the therapeutic agent that is loaded but halted in the lumen (e.g., in the form of a plug) into the middle ear; activate the therapeutic agent embedded within the lumen; release agents from the matrix and/or surface of the conduit; and/or replenish the depleted components of the therapeutic formulation (i.e., tube formulation) within the conduit matrix (FIG. 9).

In some embodiments, the priming agent B-d or a “stimulus” for C-d can activate the therapeutic agent embedded within the lumen.

In some embodiments, the priming agent B-d or a “stimulus” for C-d can release agents from the matrix and/or surface of the conduit.

In some embodiments, the priming agent B-d or a “stimulus” for C-d can replenish the depleted components of the therapeutic formulation within the conduit matrix.

In one embodiment, the inhomogeneous release of the therapeutic formulation can induce local changes in the swelling of the conduit matrix material, which further promote transport of the therapeutic agent into specific locations of the ear.

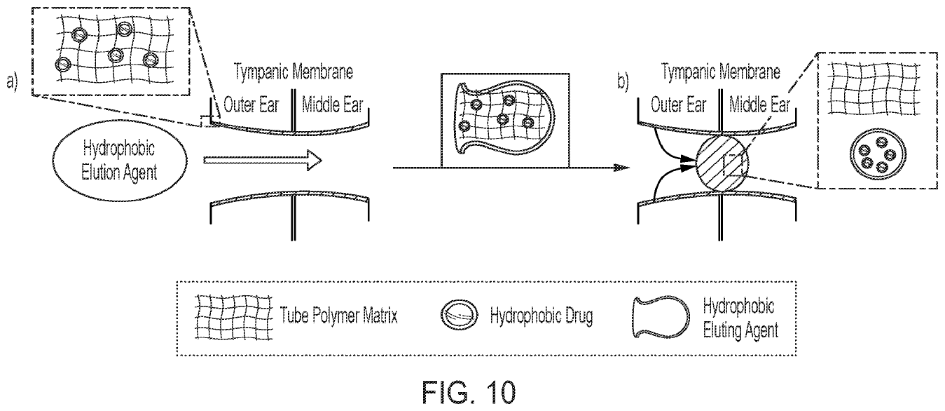

In certain embodiments, the diffusion of the therapeutic agent embedded within the conduit matrix can be enabled by the presence of immune biomarkers present within fluids in the ear space into the therapeutic agent-infused matrix. For example, diffusion of certain biomarkers from the middle ear space indicating inflammation could cause the polymer matrix to swell or collapse, thus modifying the geometry of the conduit and thus the flow profile and therapeutic agent diffusion rate. A subsequent administration of a releasing agent of a specific viscosity or surface tension could enable the release of the therapeutic agent encapsulated within the matrix. This embodiment can enable self-regulated transport of the therapeutic agent into the middle ear space to prevent overdosing or underdosing (FIG. 10).

In certain embodiments, the therapeutic agent can be introduced to the lumen of the conduit as a solid or gel form, and be solubilized by the presence of biomarkers or other fluids within the middle ear that indicate infection. This process can depend on the concentration of markers.

### (b) Case 4.2. External Manipulation of Conduit Properties During Passive Release of Therapeutic Agent Component of the Therapeutic Formulation

The conduit matrix and/or materials pre-embedded within the lumen can be made to exhibit stimuli-responsive properties. The changes in geometry and wettability in response to external fields can be used to guide the passive, sustained release of the therapeutic agent from the matrix.

In one embodiment, the therapeutic agent can be co-deposited with photothermal or magnetic nanoparticles that can be remotely actuated through light or magnetic field to heat up and facilitate transport of therapeutic agent through the matrix of the conduit. In another embodiment, the therapeutic agent can be embedded in stimuli-responsive capsules or nanoparticles within the matrix that respond to externally applied field to release the therapeutic agent, thus affecting the changes in conduit shape or wettability described above.

In another embodiment, the therapeutic agent can be co-embedded into the conduit with surfactants or other matrix-soluble compounds that can be co-released under external simulation. When released into the oil, liquid-phase, or porosity of the conduit material, these compounds can affect changes in conduit shape or wettability described above and guide the flow of the rest of the therapeutic agent. If stimuli-gradients are created, one therapeutic agent could move positively and the other one negatively along the gradient, enabling stratification of active the therapeutic agent.

In some embodiments, the active therapeutic agent can be cleaved from the dominant conduit matrix material through an externally applied stimulus, achieved through photolabile linkers, for example, and transported through diffusion in the pores of the conduit matrix. The introduction of freely suspended therapeutic agents within the conduit material can induce spatial changes in the surface properties which guide the further transport of active therapeutic agent, as described above.

In another embodiment, the actuation and manipulation of the geometry of the device via external fields can enable large spatial gradients in matrix porosity, resulting in a redirection of passive therapeutic agent transport.

E. Case 5: Design of Multifunctional Kits, Consisting of Tailored Therapeutic Agents Paired with Conduits Lacking Sustained Release Properties

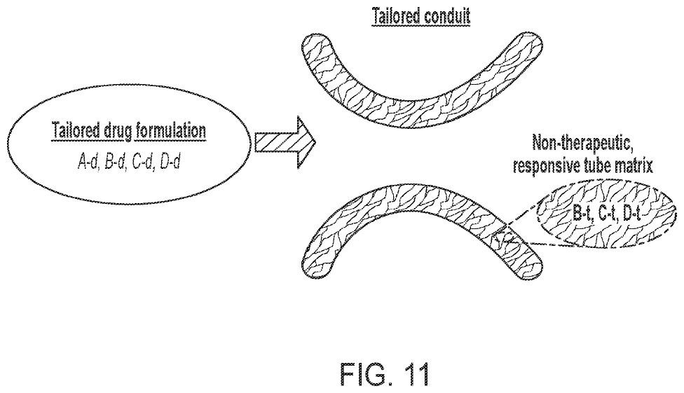

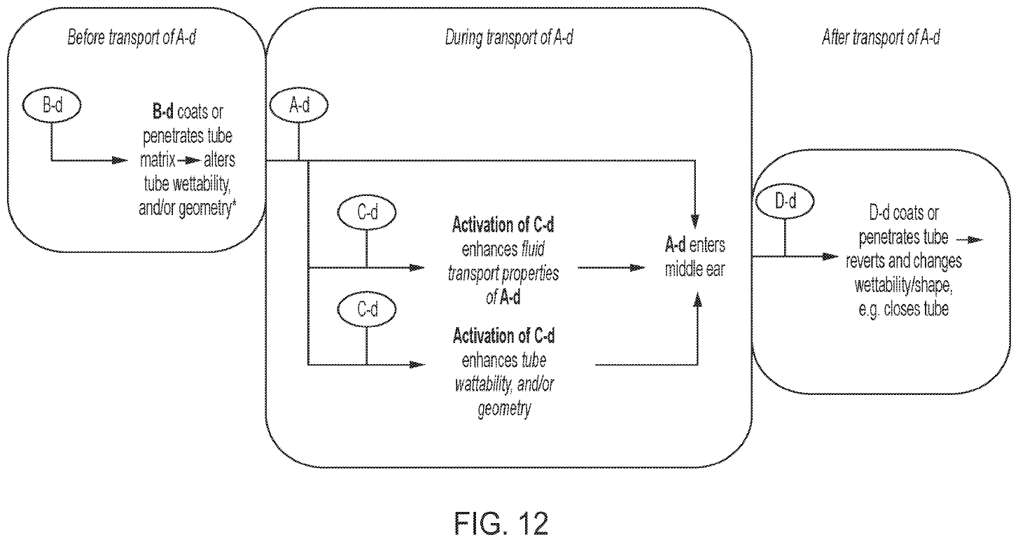

FIG. 11 shows methods for tuning either the conduit shape and/or wettability before, during and after the introduction of a drug formulation. The drug formulation can further modify the properties of the conduit to induce efficient transport. The goal is to modulate the conduit-drug formulation interaction over time to have a continuous control over the delivery of the therapeutic agent included in the drug formulation under changing use scenarios, e.g., when the patient is prescribed multiple kinds of drugs through open lumen. The flowchart shown in FIG. 12 represents many of the possible drug formulation-conduit interactions that can be designed to achieve control over the release of one or multiple therapeutic agents that are topically applied in sequence or together.

In Case 5, the conduit matrix itself does not contain embedded therapeutic formulation.

See Case 6 for embodiments in which the release and transport of the therapeutic agent can be further engineered by introduction of one or more sequential administrations of a drug formulation.

### (a) Case 5.1. Design of Conduits for Optimal Transport of a Set of Different Therapeutic Agents

(i) Case 5.1.1. Modification in Conduit Geometry and/or Wettability Immediately Prior to Administration of Each Therapeutic Agent Through the Lumen

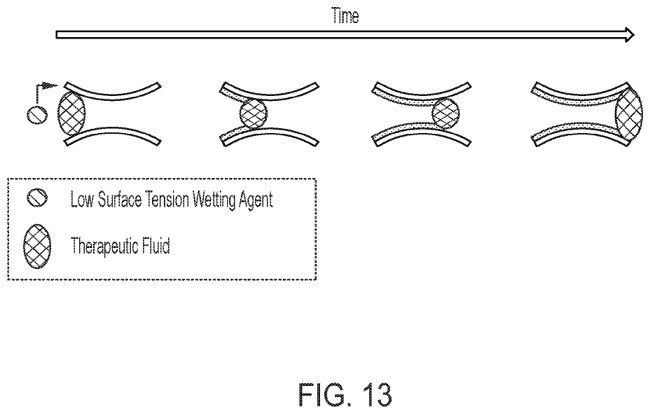

In certain embodiments, the conduit geometry and wettability can be tailored before an administration of the drug formulation to match the surface tension properties of components of the drug formulation (see FIG. 13).

The conduit wettability and geometry can be modified via introduction of a conduit lumen priming agent designed for a specific therapeutic agent. In the case of a sequential deposition, a fluid can be deposited through the lumen first to achieve easier transport of the drug formulation in various ways. In some embodiments, the first priming agent can prime the lumen with a lubricious coating to facilitate transport, or alter the pH, salt or ion concentration within the liquid component of the conduit, which induces a change in its surface wettability. Alternatively, in a specific embodiment, the introduced liquid priming agent can swell the polymer matrix to exert spatially-localized or non-localized geometry changes that modify the flow profile of subsequent fluid transport.

The transport of the drug formulations can be carried out in conjunction with a medium phase prepared independently. For instance, the medium phase can have the capability to prime the conduit for drug formulation transport in sequential deposition or modify the drug formulation transport during simultaneous deposition of both the medium phase and the active therapeutic agent.

The properties of the priming agent can further be altered through the introduction of stimuli. The stimuli can be in form of thermal energy, electromagnetics, luminescence, or chemical stimuli (pH, ion, redox, etc.). The stimuli can change the properties of the priming agent to facilitate transport of the drug formulation via favorable changes to improving wettability, lower contact angle, lower viscosity, longer slip length, or changes to the geometry by swelling the polymer dimensions.

The conduit geometry can be tailored via stimuli-responsive components in the matrix material to tailor geometry or wettability of the conduit for specific drug formulations. The conduit geometry can be changed by exposure to non-drug-related stimuli (e.g., temperature, etc.), causing swelling and/or a shape change of the conduit prior to the drug delivery.

In certain embodiments, the polymer matrix can be embedded via molecular imprinting with moieties that allow the matrix to be sensitive to biomarkers or other disease-relevant components in the ear, which induces the conduit to exhibit favorable changes to improving wettability, such as lower contact angle, lower viscosity, longer slip length, or changes to the geometry by swelling the polymer dimensions.

In certain embodiments, the therapeutic agent release can be timed. The therapeutic agent can be sequestered within the lumen of the conduit, pinned at the inlet of the conduit, or in a specific compartment within the conduit, and the release of the active therapeutic agent can be promoted at a later time via stimuli trigger. For example, the conduit can be created from stimuli-responsive materials that can be actuated to “squeeze” the fluid out of the conduit.

In some embodiments, the priming agent can be selected from the group consisting of carrier liquid for the therapeutic agent, surfactants, low-surface-energy liquids, lubricating agents, acids and bases, emulsifiers, rheology control agents, flow-promoting agents, extenders, defoaming agents, plasticizers, thickeners, heat stabilizers, porogens, levelling agents, anti-cratering agents, fillers, UV absorbers, curing agents, diluents, adjuvants, buffering agents, moistening agents, anti-oxidants, oxidizing or reducing agents, mono-dentate or multi-dentate or chelating ligands, crosslinking agents, solubilizing agents, organic and inorganic solvents, preservatives, polyglycols, long chain hydroxy polyanionic polysaccharides.

(ii) Case 5.1.2. Modification in Conduit Shape and/or Wettability Simultaneous to Administration of Drug Through the Lumen

In certain embodiments, the conduit geometry and wettability can be tailored during administration of the drug formulation to match the surface tension properties of the components of the drug formulation.

A concurrent deposition follows similar principles to sequential deposition techniques but differs in the method of application. Instead of transporting a priming agent to “prime” the conduit first, followed by the transport of the therapeutic agent in the drug formulation, both the medium phase fluid and the drug formulation are deposited together in the same fluid suspension.

In some embodiments, one could use the multifunctional priming agents that provide multifunctional actions including an ability to function as a primer and sealer in only a single coating. Such formulations when applied to a suitable substrate provide excellent adhesion to the substrate and improve performance when applied thereto.

In some embodiments, the therapeutic formulation-induced opening of pores on the surface layer of the conduit serves to prepare the surface wettability for efficient delivery. The conduit matrix becomes swollen by an externally applied lubricant and/or a therapeutic agent and/or a stimulus, resulting in opening of the pores and subsequent release of the therapeutic formulation.

Response can be designed to be specifically targeted to therapeutic agents included in existing drug formulations or components within the drug directly, both in existing drugs, and in specially tailored drug formulations that are described below.

(iii) Case 5.1.3. Modification in Conduit Shape and/or Wettability after Administration of Drug Through the Lumen

The stripping materials deposited from the drug suspensions, such as oils, including soluble components or low-volatility oils and compounds that evaporate after fluid elution, can change the surface properties of the conduit and prepare the drug for the next drug to be administered.

In some embodiments, the stripping material not only aids in preparing the drug for the next drug to be administered but it also causes the conduit to revert to the original state, preventing water or any surfactants to transport through the conduit material.

In some embodiments, nanoparticles or oil droplets are included in the therapeutic agent.

### (b) Case 5.2. Design of Tailored Drug Combinations for Optimized Transport in Conduit

### (i) Case 5.2.1. Tailoring Drug Fluid Properties Directly Before Administration of Drug Through the Lumen

In certain embodiments, the drug formulation can be modified or tailored by a medium fluid when deposited concurrently to help facilitate the transport of the therapeutic agent molecules via one of multitudes of methods: surfactant stabilization, pH gradient, concentration gradient, etc. By introduction of a gradient a stimulus can follow to induce thermophoresis, electrophoresis, or passive diffusion transport to transport active components of the drug formulation and the medium easily through the conduit.

### (ii) Case 5.2.2. Tailoring Fluid Properties of Therapeutic Agent of Drug Formulation Directly During Administration of Therapeutic Agent Through the Lumen

In certain embodiments, the drug formulation can be modified or tailored by a medium fluid when deposited sequentially to help facilitate the transport of the therapeutic agent molecules via one of multitudes of methods: surfactant stabilization, pH gradient, concentration gradient, etc.

An embodiment can be the use of a therapeutic agent component of the drug formulation for transportation with an immiscible priming agent fluid. Upon entrance of the fluids into the lumen, the immiscible fluid with higher affinity to wet the underlying conduit primes the transport of the therapeutic agent. The immiscible fluid acts as a guide transporting the therapeutic agent through the conduit.

In one embodiment, the aqueous droplet can be made to emulsify after contact with immiscible fluid present on the conduit as it passes through the lumen. This can be accomplished, for example, by designing the surface of the conduit with ridged grooves that break up the flow.

In one embodiment, active therapeutic agent molecules, inorganic or organic nanoparticles present in the therapeutic agent fluid can react chemically or biologically, directly or indirectly with both fluid or solid compounds present in the matrix of the conduit to produce a thermal, pH, or surfactant-induced gradient that inhibits or promotes flow of therapeutic agent fluid through the conduit.

In one embodiment, the drug formulation has incorporated stimuli-responsive active therapeutic agent molecules, immiscible drug delivery vehicles, or other soluble components that enables on-demand tailoring of the therapeutic agent properties directly during administration of the therapeutic agent through the lumen, by activation of stimuli-responsive materials through a pH, temperature stimulus.

In certain embodiments, a therapeutic agent is placed in a mixture with another aqueous therapeutic agent to create an emulsion of oil in water. Once this drug combination emulsion enters the conduit, the oil components wet the walls and act as a lubricious primer for the therapeutic agent to transport through the conduit. The drug combination has superior transport properties (including speed of drug elution through the lumen and volume of elution) as the emulsifying agent can have a tuned surface tension particular to the conduit geometry. This is an example of a combination of drug formulations in a concurrent transport.

(iii) Case 5.2.3. Tailoring Therapeutic Agent Properties of Drug Formulation Directly after Administration of Therapeutic Agent Through the Lumen

In certain embodiments, the drug formulation can be designed to be sequestered outside of the lumen as a pinned droplet or other morphology of the conduit via high surface tension. If required, at the requisite time, the droplet can be activated via the approaches described above to trigger transport through the conduit.

In certain embodiments, the therapeutic agent component of the drug formulation would enter the proximal end of the conduit lumen. Once the therapeutic agent fluid has entered the lumen, a drop of low surface tension fluid (surface tension lower than the infusing liquid) is added to the proximal side of the conduit (FIG. 13). Due to Marangoni effect, the introduced fluid would distribute down the surface tension gradient. Marangoni flow is enabled by realizing a surface tension gradient across an interface. There is a net force resultant in direction of higher surface tension interface. As a result, the fluid in the lumen, i.e., the therapeutic agent is also transported via this force. The fluid introduced must be immiscible with the transporting therapeutic agent fluid.

F. Case 6: Design of Multifunctional Kits, Consisting of Tailored Therapeutic Agent Component of the Drug Formulation Paired with Conduits Exhibiting Sustained Release Properties.

In this section, examples of how coupled interactions between the conduit shape and/or wettability before, during and after the introduction of a topically applied therapeutic agent through the length of the conduit are described. As in Case 4, the conduit matrix itself contains embedded therapeutic agent components which can further interact physically, chemically, or concurrently with some stimuli with the incoming topical therapeutic agent contained in the drug formulation (see FIG. 2). FIG. 3 shows a flowchart of a possible drug-conduit interaction to achieve (a) a sustained therapeutic agent release and enhanced drug formulation flux through a sustained release from the conduit of one or more compounds, (b) a tailoring of favorable interactions between a drug formulation and therapeutic formulation embedded in the conduit as part of the matrix or as part of the lumen, and (c) replenishment of the therapeutic formulation components after their use.

Certain embodiments utilize multiplexed and tunable interactions between the conduit (conduit solid matrix, conduit-embedded empty space, e.g., pores, vasculature) and the therapeutic agent component of the drug formulation (active therapeutic agent molecules, e.g., prodrug, codrug, antibiotics, antibodies.), drug delivery vehicles—organic/inorganic nanomaterials (e.g., liposomes or nanotubes), immiscible oil (e.g., nanoemulsion, microemulsion), carrier liquid for the therapeutic agent and other soluble components (e.g., surfactants), infused liquid, other cleavable or non-covalently attached molecules (e.g., drug or other component).

(a) Case 6.1. Embedding Therapeutic Agents or Delivery Vehicles within the Conduit Material or Conduit Lumen that Interact Physically with Therapeutic Agent or Releasing Agent Components of Drug Formulation

In some embodiments, the conduit can be self-priming. The conduit matrix can be embedded with one or multiple fluids (e.g., oil) or vapor component fluid components that, upon release, create gradients in surface tension, pH, release, heat (via exothermic reactions) that further guide the diffusion, elution and spreading of the therapeutic agent components of the drug formulation.

In some embodiments, a hydrophobic therapeutic agent can be released from within the conduit matrix during administration of topical therapeutic. One therapeutic agent component of the therapeutic formulation tailored for sustained release can be infused into the conduit matrix and can be released upon administration of the topically administered therapeutic or releasing agent components of the drug formulation. The therapeutic agent fluid or releasing agent components of the drug formulation can be an oil that allows the hydrophobic drug to dissolve into the new medium phase from the conduit matrix and transport into the middle ear space.

In certain embodiments, the releasing agent can consist of a solvent that swells part of the matrix of the conduit and allows for faster diffusion of the therapeutic agent molecules through the expanded pores of the matrix. The mesh size of the matrix can be tuned through the swelling ratio induced by the introduction of the second component to allow for quick short-term release or slower long-term release.

Depending on the interplay between the geometric response of the conduit to the carrier fluid for the therapeutic agent (for example through conduit swelling and “pinching” of the conduit lumen, or other similar response), the embedded therapeutic agent within different sections of the conduit can be either released or retained to promote targeted and localized drug delivery.

The conduit geometry matrix can be infused with an oil. Upon introducing a therapeutic agent component of the therapeutic formulation into the conduit with another oil (a drug emulsion combination), the matrix oil is replaced by the emulsion oil. This replacement of oil in the matrix of the conduit drives excess oil to the walls of the conduit. This takes advantage of pre-existing oil within the matrix of the conduit to act as a lubricant for the transport of the therapeutic agent. This embodiment allows therapeutic agent to be used with a compatible oil combination (certain oils form micelles at lower concentrations and are more stable but provide lower lubricity). Whereas, the conduit matrix can contain more lubricous oils tuned for the conduit geometry. This multicomponent system enables a wide array of aqueous therapeutics to be transported by an oil combination (one oil mixed with the embedded therapeutic agent and one residing in the conduit matrix). Once the therapeutic agent is transported, the lubricious oil can be replenished into the conduit matrix. The displacement of matrix oil is driven by the higher affinity of the emulsion oil for the conduit matrix material.

(b) Case 6.2. Embedding Therapeutic Agent Component of Therapeutic Formulation within the Conduit Matrix that Interacts Chemically with Therapeutic Agent or Releasing Agent Component of Drug Formulation

In certain embodiments, a second chemical compound, contained within the therapeutic agent of the therapeutic formulation, may be introduced to the conduit, which will simultaneously activate the therapeutic agent through swelling induced via cleavage, disassembly, gas generation or cap removal mechanisms.

An embodiment can be the reaction of one component within the administered therapeutic agent component of the drug formulation with a chemical component embedded on the surface of conduit matrix to form a mildly exothermic reaction, which guides the transport of the second therapeutic component via thermophoresis and promotes drug diffusion.

The conduit can contain a catalyst or enzyme within the liquid or solid portions of the conduit material, which further promotes the activation, association or compounds or other functional therapeutic agent delivery.

(c) Case 6.3. Embedding Therapeutic Agent Component of Therapeutic Formulation within the Conduit Matrix that Interacts, Together with an Externally Applied Stimulus, with Topically Applied Therapeutic Agent or Releasing Agent Components of Drug Formulation

In another embodiment, parts of the conduit can be infused with the therapeutic agents with photosensitizer catalysts or molecules, which can become active under remotely applied stimuli or cleaved and become active with a stimulus, e.g., near infrared light, respectively.

In certain embodiments, multiple types of therapeutic agent-harboring compounds can be simultaneously embedded into the matrix material through direct incorporation or through a cleavable, gas generating, or capped vehicle. Subsequently, the components can be cleaved via releasing agent(s) or the matrix material can be swollen with oil to release compounds sequentially and in an as-needed fashion.

Combinatorial approaches for addressing all scenarios shown in FIG. 2 can be created by combining tailored therapeutic agents and conduits that change surface properties and shape because of external effects and/or therapeutic agents.

G. Case 7: Multifunctional Drug Formulation-Conduit Kits with on-Demand Opening and Closure Mechanisms

In some embodiment, the conduit lumen can have a mechanism of on-demand closing or opening the conduit that releases the drug formulation either passively, under stimulus, or upon introduction of a drug formulation.

In one embodiment, the therapeutic formulation in a solid or plug formulation in gel “plug” form can be initially embedded into the lumen of the conduit and solubilized or released by the introduction of a drug formulation; or propelled by a jet stream, ultrasound, or compressed air; the therapeutic formulation in a solid or plug formulation can be electroporated; or plug formulation can be vaporized with the release of therapeutic agent into the ear. Furthermore, the release of this plug can be enhanced through the shape changes or wettability changes that can be induced by embodiments shown FIG. 2.

In some embodiments, the conduit or plug comprises a chemical, magnetic, electrical or optical sensor that measures the amount of each of component of the drug formulation that is delivered through the conduit and provides a readout. The sensor can: (1) specifically detect the concentration of a particular agent, and (2) report how much of the agent actually reached the middle ear (e.g., via microelectronics or colorimetric methods).

A series of experiments, including a lab-scale in vitro and an in vivo model, were conducted to establish the feasibility, efficacy and reliability of the therapeutic agent delivery using the optimized drug formulation-conduit (i.e., kit) approach. The details of the experimental setup and results are discussed below.

### 1. In Vitro Assessment of Therapeutic Agent Transport Using Kit Approach

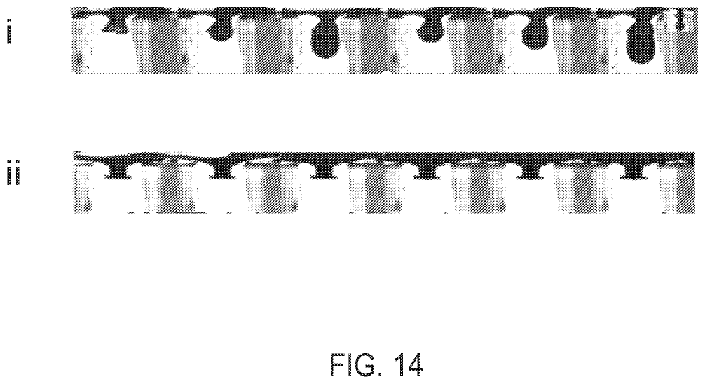

One experiment focused on validating the transport of the drug, i.e., therapeutic agent as it is deposited on the proximal end of the optimized conduit. The experiment was also performed with a conduit having a conventional cylindrical geometry for comparison purposes. The therapeutic agent used for testing was Ciprodex® (ciprofloxacin 0.3% and dexamethasone 0.1% otic suspension), which is an antibiotic-containing therapeutic that is commonly prescribed after TT implantation and has proven safe and effective for eradicating common pathogens. A droplet of Ciprodex® (applied volume of 250 μL) was applied to the proximal end of the lumen of the conduits, and the transport behavior of the droplet through the conduit lumen was studied as shown in FIG. 14. The panels (i) and (ii) of FIG. 14 correspond to the optimized conduit and conventional conduit, respectively. FIG. 14 demonstrates that the optimized conduit of the optimized therapeutic agent-conduit combination allows for an immediate transport of Ciprodex® droplet (as shown in panel (i) of FIG. 14), in contrast to the conventional conduit that does not allow for the transport of Ciprodex® (as shown in panel (ii) of FIG. 14).

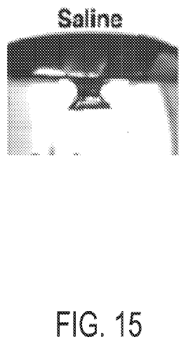

To examine the efficacy of the optimized therapeutic agent-conduit combination in preventing the water transport, a saline water droplet was applied to the optimized conduit. As is evident from FIG. 15, the optimized curved geometry of the conduit does not allow for any transport of water.

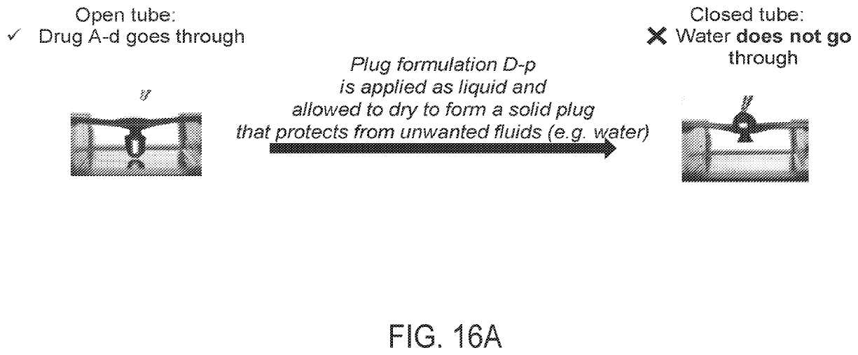

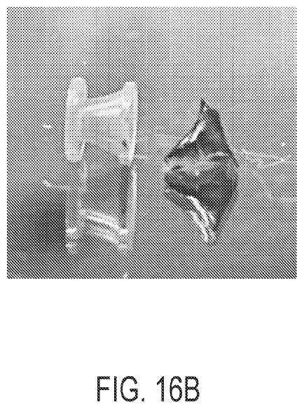

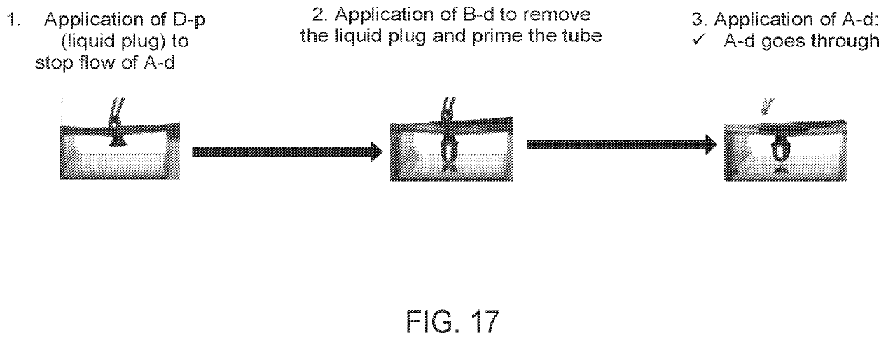

Another set of experiments (FIG. 16 and FIG. 17) has examined the efficacy of introducing a solid or liquid plug over the inner surface of the conduit to modify the transport of the unwanted fluids, such as environmental water, or drugs.

FIG. 16A demonstrates how a solid biodegradable or non-biodegradable plug (e.g., made of egg whites) can preclude water or other fluids from flowing through the tube. The solid plug can then be removed on demand to restore the fluid transport capabilities (FIG. 16B).

FIG. 17 demonstrates how a liquid plug rapidly formed on demand by a high-viscosity fluid (e.g., silicone fluid) allows for prevention of transport of A-d therapeutic component. The priming agent B-d (e.g., low-viscosity silicone fluid) can then be introduced to quickly remove the liquid plug on demand and prime the tube for the consequent therapeutic transport of A-d.

### 2. In Vivo Assessment of Therapeutic Agent Transport Using Kit Approach

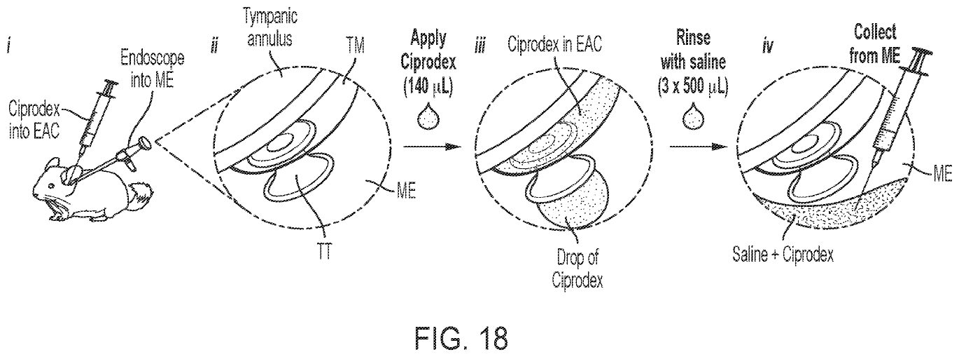

A separate experiment tested the feasibility of the optimized drug-conduit combination approach in vivo in a chinchilla model, as schematically shown in the FIG. 18. Chinchillas represent a gold-standard model in the course of otitis media in humans. The biocompatibility and surgical handleability of the optimized tympanostomy conduits through implantation experiments in a chinchilla model have been confirmed as a first step. The feasibility of the drug transport function of the optimized conduits has been demonstrated when they are implanted within the physiological environment of the ear. The optimized conduit approach involved using an optimized liquid-infused tympanostomy tubes (TTs) with inner diameter ID=0.66 mm, termed “LI-TTs” that were implanted into the eardrum of healthy chinchillas (a representative schematic shown in panel (i) of FIG. 18) and compared to a commonly implanted state-of-the-art TT (Summit Medical VT-1002-01, sterile commercial silicone Collar Button, ID=1.27 mm, “SM-TTs”) (a representative schematic shown in panel (ii) of FIG. 18). The drug transport efficacy was assessed in the absence of pathogenic bacteria or infection. An antibiotic Ciprodex® was used as a therapeutic agent. It is typically administered in 7-day regimens, 2 doses a day at ˜140 μl per dose. As antibiotic droplets do not spontaneously enter TTs, patients are recommended to pump the tragus 5 times to coerce therapeutics to enter the middle ear and lie with their ears upright for at least 1 min after drug administration. This approach relies exclusively on patient training and compliance, and does not provide guarantee of therapeutic delivery into the middle ear.

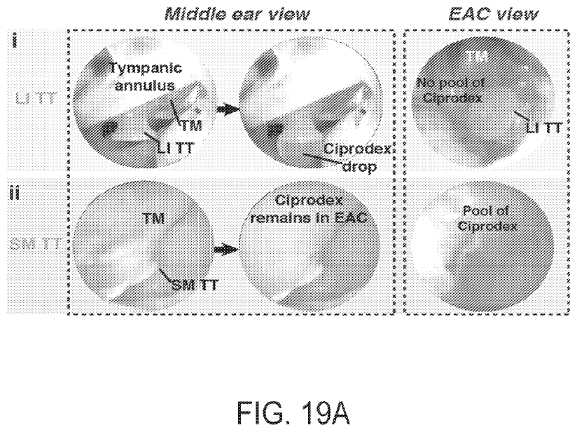

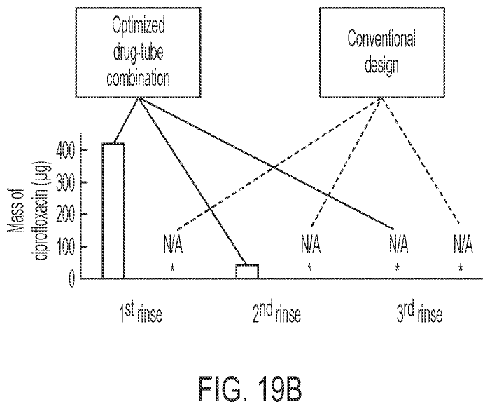

To demonstrate superiority of the optimized LI-TTs for improving drug transport reliability and efficacy, we assessed the feasibility of an increase in active drug component ciprofloxacin transported to the middle ear through implantation of LI-TTs compared to SM-TTs without tragal pumping. We have conducted preliminary work to visualize and assess feasibility of quantitatively comparing drug transport through different TTs implanted in healthy chinchillas as shown in the FIG. 18 and FIG. 19. The results from endoscopic visualization from within the middle ear on the drug elution replicate results from in vitro studies (FIG. 19A (i) and (ii)). The time to elution of Ciprodex® was defined as the timepoint at which the droplet detached from the tube into the middle ear and could be quantitatively captured through the videos. Endoscopic images obtained from the middle ear side suggest that test TTs promote earlier and more reliable transport of therapeutic droplets compared to control Summit Medical tubes.

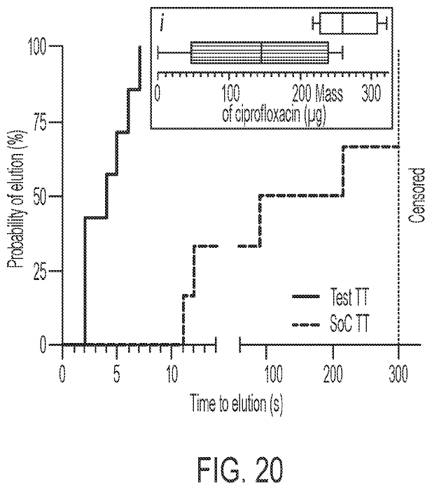

Our data, shown in FIG. 20, clearly reveals that Ciprodex® droplets take only 2-7 s to reach the middle ear through 100% of test TTs, while the standard-of-care tubes take up to 300 s or longer to elute. Thus, there is a significant decrease in the time that it takes for ciprofloxacin to be transported through optimized TTs compared to SoC TTs (p=0.0001, nonparametric Mantel-Cox log rank test). Based on these results, protocols have been developed for assessing the final Ciprodex® content in the middle ear using middle ear fluid extraction, high performance liquid chromatography (HPLC) and mass spectrometry (MS) methods. Chinchillas were randomly allocated into two groups (LI-TT, denoted as Test TT, and SM-TT, denoted as SoC TT). Middle ear fluid obtained from all ears was analyzed for Ciprodex® concentration (or mass) using HPLC and MS, and the results are plotted in the FIG. 20. The mass of ciprofloxacin present within the middle ear after 5 minutes of therapeutic delivery was up to 5 times higher for animals implanted with test tubes compared to SoC TTs (p=0.027, Mann Whitney test).

1-236. (canceled)

237. A method comprising:
administering a drug formulation to the middle ear of an ear by depositing the drug formulation into a conduit positioned in the ear, wherein
the drug formulation comprises a therapeutic agent, and
the conduit comprises:
a proximal end, the proximal end having a proximal end radius;
a distal end opposite the proximal end, the distal end having a distal end radius;
an inner surface connecting the proximal end and the distal end, at least a portion of the inner surface comprising a conical or curved geometry extending at least partially between the proximal end and the distal end;
an outer surface connecting the proximal end and the distal end, and
the conduit is configured to allow a selective transport of the therapeutic agent from the proximal end to the distal end of the conduit.

238. The method of claim 237, wherein the proximal end is disposed in an ear canal of the ear and the distal end is disposed in the middle ear.

239. The method of claim 237, wherein a shape of the conduit is selected from a group consisting of a tube, a shell, a channel, a hose, a pipe, a cone, an oval, and a combination thereof.

240. The method of claim 237, wherein the conduit comprises one or more flanges.

241. The method of claim 237, wherein the conduit is configured to be self-supporting, bendable, shape retaining, or deformable.

242. The method of claim 237, wherein the conduit comprises one or more of nano-sized, micro-sized, or millimeter-sized pores.

243. The method of claim 237, wherein the conduit is configured to prevent transport of an infectious material from the proximal end to the distal end of the conduit.

244. The method of claim 237, wherein the conduit is configured to transport a biofluid of the middle ear from the distal end to the proximal end of the conduit.

245. The method of claim 237, wherein the distal end radius is equal to or larger than the proximal end radius.

246. The method of claim 237, wherein the drug formulation further comprises one or more of a priming agent and an activating agent,
wherein the priming agent modifies one or more properties of the conduit from a first configuration to a second configuration either upon activation of the priming agent or caused by one or more of wetting and infusion of the priming agent, and
wherein the activating agent is capable of releasing the therapeutic agent toward a portion of the inner surface of the conduit upon activation of the activating agent.

247. The method of claim 237, wherein the conduit includes a lubricating fluid formed over at least a portion of the inner surface and the outer surface of the conduit.

248. The method of claim 247, wherein the lubricating fluid comprises a silicone oil.

249. The method of claim 247, wherein the lubricating fluid comprises a fluid selected from a group consisting of fluocinolone acetonide oil, fully or partially fluorinated hydrocarbons, tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, or a combination thereof.

250. The method of claim 237, wherein the therapeutic agent comprises a solution, a suspension, or a combination thereof.

251. The method of claim 237, wherein the therapeutic agent comprises an antimicrobial or antifungal agent selected from a group consisting of ciprofloxacin, ofloxacin, clotrimazole, miconazole, bicyclic heteroarylsubstituted imidazole compound, and a combination thereof.

252. The method of claim 237, wherein the therapeutic agent comprises an antiviral agent selected from a group consisting of acyclovir, famciclovir, valacyclovir, and a combination thereof.

253. The method of claim 237, wherein the therapeutic agent comprises a steroid selected from a group consisting of corticosteroids, dexamethasone, prednisone, cortisone, and a combination thereof.

254. The method of claim 237, wherein the therapeutic agent comprises an ion channel modulator selected from a group consisting of ampicillin, calcium channel blockers, diltiazem, nifedipine, verapamil, drugs that act on sodium channels, riluzole, dextromethorphan, lidocaine, and a combination thereof

255. The method of claim 237, wherein the therapeutic agent is selected from a group consisting of toll-like receptor inhibitors, TNF-α inhibitors, interleukin inhibitors, calcineurin inhibitors, and a combination thereof.

256. The method of claim 237, wherein the therapeutic agent comprises a surfactant selected from a group consisting of phospholipids, cholesterol, and a combination thereof.

257. The method of claim 246, wherein the priming agent comprises a lubricating liquid.

258. The method of claim 257, wherein the lubricating liquid comprises a silicone oil.

259. The method of claim 257, wherein the lubricating liquid comprises a fluid selected from a group consisting of fluocinolone acetonide oil, fully or partially fluorinated hydrocarbons, tertiary perfluoroalkylamines, perfluoroalkylsulfides, perfluoroalkylsulfoxides, perfluoroalkylethers, perfluorocycloethers, perfluoropolyethers, perfluoroalkylphosphines, or perfluoroalkyl phosphine oxides, or a combination thereof.

260. The method of claim 237, wherein the conduit comprises a material selected from a group consisting of silicone, polysiloxane, or a combination thereof.

261. The method of claim 237, wherein the conduit comprises a material selected from a group consisting of fluoropolymer, polyvinyl fluoride, polytetrafluorethylenes, fluoroplastics, polyvinylidene fluoride, polytetrafluoroethylene, and a combination thereof.
